SEQUENCE LISTING (14 sequences)

SEQ ID NO: 1 - DNA, 6129 bp, Homo sapiens
aattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatattacaatgctgtattttaaaagaatgattatgaattacatttgtataaaataatttttatatttgaaatattgactttttatggcactagtatttttatgaaatattatgttaaaactgggacaggggagaacctagggtgatattaaccaggggccatgaatcaccttttggtctggagggaagccttggggctgatcgagttgttgcccacagctgtatgattcccagccagacacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgt

SEQ ID NO: 2 - DNA, 22 bp, Artificial sequence
  Single strand DNA oligonucleotide
gcaccattaaagaaaatatgat

SEQ ID NO: 3 - DNA, 19 bp, Artificial sequence
  Single strand DNA oligonucleotide
ctcttctagttggcatgct

SEQ ID NO: 4 - DNA, 20 bp, Artificial sequence
  Single strand DNA oligonucleotide
taatggatcatgggccatgt

SEQ ID NO: 5 - DNA, 20 bp, Artificial sequence
  Single strand DNA oligonucleotide
acagtgttgaatgtggtgca

SEQ ID NO: 6 - DNA, 16 bp, Artificial sequence
  Single strand DNA oligonucleotide
gttgttggaggttgct

SEQ ID NO: 7 - DNA, 16 bp, Artificial sequence
  Single strand DNA oligonucleotide
gttgttggcggttgct

SEQ ID NO: 8 - DNA, 20 bp, Artificial sequence
  Single strand DNA oligonucleotide
gcagagtacctgaaacagga

SEQ ID NO: 9 - DNA, 20 bp, Artificial sequence
  Single strand DNA oligonucleotide
ggcataatccaggaaaactg

SEQ ID NO: 10 - DNA, 20 bp, Artificial sequence
  Single strand DNA oligonucleotide
ggcataatccaggaaaacta

SEQ ID NO: 11 - DNA, 22 bp, Artificial sequence
  Single strand DNA oligonucleotide
ccgactcgagnnnnnnatgtgg

SEQ ID NO: 12 - RNA, 50 nt, Artificial sequence
  In situ hybridization probe
cguaauggaaugcuugaaggcugcuccgugaugucggucggagcuuccag

SEQ ID NO: 13 - DNA, 28 bp, Artificial sequence
  Single strand DNA oligonucleotide
gaaactggcctccaaacactgcccgccg

SEQ ID NO: 14 - DNA, 29 bp, Artificial sequence
  Single strand DNA oligonucleotide
gtcttgttggagatgcacgtgccccttgc

(19) United States
(12) Patent Application Publication
(10) Pub. No.: US 2008/0261822 A1
(43) Pub. Date: Oct. 23, 2008

(54) Non-invasive prenatal genetic diagnosis using transcervical cells

(76) Inventors: Moshe D. Fejgin, Tel-Aviv (IL); Aliza Amiel, Ein-Sarid (IL); Meytal Liberman, Rehovot (IL)

Correspondence Address: Gary M. Nath;THE NATH LAW GROUP, 112 South West Street, Alexandria, VA, 22314, US

(21) Appl. No.: 11/709,794

(22) Filed: Feb. 23, 2007

Related U.S. Application Data
(62) Division of application No. 11/088,882, filed on Mar. 25, 2005.
Application No. 11/088,882 is a continuation-in-part of application No. 10/921,899, filed on Aug. 20, 2004.
Application No. 10/921,899 is a continuation-in-part of application No. PCT/IL2004/000304, filed on Apr. 1, 2004.

(51) Int. Cl. C40B 30/04; C12Q 1/68

(52) U.S. Cl. 506/9; 435/6

(57) ABSTRACT

A non-invasive, risk-free method of prenatal diagnosis is provided. According to the method of the present invention transcervical specimens are subjected to trophoblast-specific immuno-staining followed by FISH, PRINS, Q-FISH and/or MCB analyses and/or other DNA-based genetic analysis in order to determine fetal gender and/or identify chromosomal and/or DNA abnormalities in a fetus. Also provided is a method of in situ chromosomal, DNA and/or RNA analysis of a prestained specimen by incubating the prestained specimen in ammonium hydroxide. Also provided is a method of identifying embryonic cells according to a nucleus/cytoplasm ratio of at least 1:1 and the presence of at least variably condensed chromatin.

This application is a continuation-in-part of U.S. patent application Ser. No. 10/921,899, filed Aug. 20, 2004, which is a continuation-in-part of PCT Patent Application No. PCT/IL2004/000304, filed Apr. 1, 2004, which claims the benefit of priority from U.S. patent application Ser. No. 10/405,698, filed Apr. 3, 2003, now abandoned. The contents of the above applications are hereby incorporated by reference in their entirety.

### FIELD AND BACKGROUND OF THE INVENTION

The present invention relates to a method of diagnosing genetic abnormalities using trophoblast cells from transcervical specimens, and, more particularly, to the biochemical and genetic analysis of trophoblast cells for determination of fetal gender and/or chromosomal abnormalities in a fetus.

Prenatal diagnosis involves the identification of major or minor fetal malformations or genetic diseases present in a human fetus. Ultrasound scans can usually detect structural malformations such as those involving the neural tube, heart, kidney, limbs and the like. On the other hand, chromosomal aberrations such as presence of extra chromosomes [e.g., Trisomy 21 (Down syndrome); Klinefelter's syndrome (47, XXY); Trisomy 13 (Patau syndrome); Trisomy 18 (Edwards syndrome); 47, XYY; 47, XXX], the absence of chromosomes [e.g., Turner's syndrome (45, X0)], or various translocations and deletions can be currently detected using chorionic villus sampling (CVS) and/or amniocentesis.

Currently, prenatal diagnosis is offered to women over the age of 35 and/or to women which are known carriers of genetic diseases such as balanced translocations or microdeletions (e.g., Angelman syndrome), and the like. Thus, the percentage of women over the age of 35 who give birth to babies with chromosomal aberrations to such as Down syndrome has drastically reduced. However, the lack of prenatal testing in younger women resulted in the surprising statistics that 80% of Down syndrome babies are actually born to women under the age of 35.

CVS is usually performed between the 9th and the 14th week of gestation by inserting a catheter through the cervix or a needle into the abdomen and removing a small sample of the placenta (i.e., chorionic villus). Fetal karyotype is usually determined within one to two weeks of the CVS procedure. However, since CVS is an invasive procedure it carries a 2-4% procedure-related risk of miscarriage and may be associated with an increased risk of fetal abnormality such as defective limb development, presumably due to hemorrhage or embolism from the aspirated placental tissues (Miller D, et al, 1999. Human Reproduction 2: 521-531).

On the other hand, amniocentesis is performed between the 16^{th} to the 20^{th} week of gestation by inserting a thin needle through the abdomen into the uterus. The amniocentesis procedure carries a 0.5-1.0% procedure-related risk of miscarriage. Following aspiration of amniotic fluid the fetal fibroblast cells are further cultured for 1-2 weeks, following which they are subjected to cytogenetic (e.g., G-banding) and/or FISH analyses. Thus, fetal karyotype analysis is obtained within 2-3 weeks of sampling the cells. However, in cases of abnormal findings, the termination of pregnancy usually occurs between the 18th to the 22nd week of gestation, involving the Boero technique, a more complicated procedure in terms of psychological and clinical aspects.

To overcome these limitations, several approaches of identifying and analyzing fetal cells using non-invasive procedures were developed.

One approach is based on the discovery of fetal cells such as fetal trophoblasts, leukocytes and nucleated erythrocytes in the maternal blood during the first trimester of pregnancy. However, while the isolation of trophoblasts from the maternal blood is limited by their multinucleated morphology and the availability of antibodies, the isolation of leukocytes is limited by the lack of unique cell markers which differentiate maternal from fetal leukocytes. Moreover, since leukocytes may persist in the maternal blood for as long as 27 years (Schroder J, et al., 1974. Transplantation, 17: 346-360; Bianchi D W, et al., 1996. Proc. Natl. Acad. Sci. 93: 705-708), residual cells are likely to be present in the maternal blood from previous pregnancies, making prenatal diagnosis on such cells practically impossible.

On the other hand, nucleated red blood cells (NRBCs) have a relatively short half-life of 90 days, making them excellent candidates for prenatal diagnosis. However, several studies have found that at least 50% of the NRBCs isolated from the maternal blood are of maternal origin (Slunga-Tallberg A et al., 1995. Hum Genet. 96: 53-7). Moreover, since the frequency of nucleated fetal cells in the maternal blood is exceptionally low (0.0035%), the NRBC cells have to be first purified (e.g., using Ficol-Paque or Percoll-gradient density centrifugation) and then enriched using e.g., magnetic activated cell sorting (MACS, Busch, J. et al., 1994, Prenat. Diagn. 14: 1129-1140), ferrofluid suspension (Steele, C. D. et al., 1996, Clin. Obstet. Gynecol. 39: 801-813), charge flow separation (Wachtel, S. S. et al., 1996, Hum. Genet. 98:162-166), or FACS (Wang, J. Y. et al., 2000, Cytometry 39:224-230). However, such purification and enrichment steps resulted in inconsistent recovery of fetal cells and limited sensitivity in diagnosing fetal's gender (reviewed in Bischoff, F. Z. et al., 2002. Hum. Repr. Update 8: 493-500). Thus, the combination of technical problems, high-costs and the uncertainty of the origin of the cells have prevented this approach from actually becoming clinically accepted.

Another approach is based on the presence of trophoblast cells (shed from the placenta) in the cervical canal [Shettles LB (1971). Nature London 230:52-53; Rhine S A, et al (1975). Am J Obstet Gynecol 122:155-160; Holzgreve and Hahn, (2000) Clin Obstet and Gynaecol 14:709-722]. Trophoblast cells can be retrieved from the cervical canal using (i) aspiration; (ii) cytobrush or cotton wool swabs; (iii) endocervical lavage; or (iv) intrauterine lavage.

Once obtained, the trophoblastic cells can be subjected to various methods of determining genetic diseases or chromosomal abnormalities.

Griffith-Jones et al, [British J Obstet. and Gynaecol. (1992). 99: 508-511) presented PCR-based determination of fetal gender using trophoblast cells retrieved with cotton wool swabs or by flushing of the lower uterine cavity with saline. However, this method was limited by false positives as a result of residual semen in the cervix. To overcome these limitations, a nested PCR approach was employed on samples obtained by mucus aspiration or by cytobrush. These analyses resulted in higher success rates of fetal sex prediction (Falcinelli C., et al, 1998. Prenat. Diagn. 18: 1109-1116). However, direct PCR amplifications from unpurified transcervical cells are likely to result in maternal cell contamination.

A more recent study using PCR and FISH analyses on transcervical cells resulted in poor detection rates of fetal gender (Cioni R., et al, 2003. Prenat. Diagn. 23: 168-171).

Therefore, to distinguish trophoblast cells from the predominant maternal cell population in transcervical cell samples, antibodies directed against placental antigens were employed.

Miller et al. (Human Reproduction, 1999. 14: 521-531) used various trophoblast-specific antibodies (e.g., FT1.41.1, NCL-PLAP, NDOG-1, NDOG-5, and 340) to identify trophoblast cells from transcervical cells retrieved using transcervical aspiration or flushing. These analyses resulted in an overall detection rate of 25% to 79%, with the 340 antibody being the most effective one.

Another study by Bulmer, J. N. et al., (Prenat. Diagn. 2003. 23: 34-39) employed FISH analysis in transcervical cells to determine fetal gender. In this study, all samples retrieved from mothers with male fetuses found to contain some cells with Y-specific signals. In parallel, duplicated transcervical samples were subjected to IHC using a human leukocyte antigen (HLA-G) antibody (G233) which can recognize all populations of extravillous trophoblasts (Loke, Y. W., et al., 1997. Tissue Antigen 50: 135-146; Loke and King, 2000, Ballieres Best Pract Clin Obstet Gynaecol 14: 827-837). HLA-G positive cells were present in 50% of the samples (Bulmer, J. N. et al., (2003) supra). However, since the FISH analysis and the trophoblast-specific IHC assay were performed on separated slides, it was impractical to use this method for diagnosing fetal chromosomal abnormalities.

There is thus a widely recognized need for, and it would be highly advantageous to have, a method of determining fetal gender and/or identifying chromosomal abnormalities in a fetus devoid of the above limitations.

### SUMMARY OF THE INVENTION

According to one aspect of the present invention there is provided a method of determining fetal gender and/or identifying at least one chromosomal abnormality of a fetus: (a) immunologically staining a trophoblast-containing cell sample to thereby identify at least one trophoblast cell, and (b) subjecting the at least one trophoblast cell to in situ chromosomal and/or DNA analysis to thereby determine fetal gender and/or identify at least one chromosomal abnormality.

According to another aspect of the present invention there is provided a method of determining fetal gender and/or identifying at least one chromosomal and/or DNA abnormality of a fetus: (a) immunologically staining a trophoblast-containing cell sample to thereby identify at least one trophoblast cell; (b) subjecting the at least one trophoblast cell to in situ chromosomal and/or DNA analysis to thereby obtain at least one stained trophoblast cell, and; (c) subjecting at least one stained trophoblast cell to a genetic analysis to thereby determine fetal gender and/or identify at least one chromosomal and/or DNA abnormality.

According to yet another aspect of the present invention there is provided a method of determining fetal gender and/or identifying at least one chromosomal abnormality of a fetus: (a) immunologically staining a trophoblast-containing cell sample to thereby identify at least one trophoblast cell, and; (b) subjecting the at least one trophoblast cell to a genetic analysis to thereby determine fetal gender and/or identify at least one chromosomal abnormality.

According to still another aspect of the present invention there is provided a method of determining fetal gender and/or identifying at least one chromosomal abnormality of a fetus, comprising sequentially subjecting a trophoblast-containing cell sample to an RNA-in situ hybridization (RNA-ISH) staining and an in situ chromosomal and/or DNA analysis to thereby determine fetal gender and/or identify at least one chromosomal abnormality.

According to an additional aspect of the present invention there is provided a method of determining fetal gender and/or identifying at least one chromosomal and/or DNA abnormality of a fetus, comprising: (a) simultaneously or sequentially subjecting a trophoblast-containing cell sample to an RNA-in situ hybridization (RNA-ISH) staining and an in situ chromosomal and/or DNA analysis to thereby obtain at least one stained trophoblast cell and; (b) subjecting the at least one stained trophoblast cell to a genetic analysis to thereby determine fetal gender and/or identify at least one chromosomal and/or DNA abnormality.

According to yet an additional aspect of the present invention there is provided a method of determining fetal gender and/or identifying at least one chromosomal and/or DNA abnormality of a fetus, comprising: (a) subjecting a trophoblast-containing cell sample to an RNA-in situ hybridization (RNA-ISH) staining to thereby obtain at least one stained trophoblast cell, and; (b) subjecting the at least one stained trophoblast cell to a genetic analysis to thereby determine fetal gender and/or identify at least one chromosomal and/or DNA abnormality.

According to still an additional aspect of the present invention there is provided A method of determining a paternity of a fetus, comprising: (a) subjecting a trophoblast-containing cell sample to an RNA-in situ hybridization (RNA-ISH) staining to thereby obtain at least one stained trophoblast cell; (b) subjecting the at least one stained trophoblast cell to a genetic analysis to thereby identify polymorphic markers of the fetus, and; (c) comparing the identified polymorphic markers of the fetus to a set of polymorphic markers obtained from at least one potential father to thereby determine the paternity of the fetus.

According to a further aspect of the present invention there is provided a method of determining a paternity of a fetus, comprising: (a) immunologically staining a trophoblast-containing cell sample to thereby identify at least one trophoblast cell, and; (b) subjecting the at least one stained trophoblast cell to a genetic analysis to thereby identify polymorphic markers of the fetus, and; (c) comparing the identified polymorphic markers of the fetus to a set of polymorphic markers obtained from a potential father to thereby determine the paternity of the fetus.

According to yet a further aspect of the present invention there is provided a method of in situ chromosomal, DNA and/or RNA analysis by hybridization of a prestained specimen of cells or tissue, comprising incubating the prestained specimen of cells or tissue in a solution containing ammonium hydroxide and thereafter incubating the prestained specimen of cells or tissue with a polynucleotide probe suitable for the in situ chromosomal, DNA or RNA analysis.

According to still a further aspect of the present invention there is provided a method of identifying an embryonic cell in a mixed cell population, the method comprising identifying a cell exhibiting: (i) a nucleus/cytoplasm ratio of at least 1:1; and (ii) at least variably condensed chromatin, thereby identifying the embryonic cell in the mixed cell population.

According to further features in preferred embodiments of the invention described below, the trophoblast-containing cell sample is obtained from a cervix and/or a uterine.

According to still further features in the described preferred embodiments the trophoblast-containing cell sample is obtained using a method selected from the group consisting of aspiration, cytobrush, cotton wool swab, endocervical lavage and intrauterine lavage.

According to still further features in the described preferred embodiments the trophoblast cell sample is obtained from a pregnant woman at 6^{th} to 15^{th} week of gestation.

According to still further features in the described preferred embodiments the trophoblast cell sample is obtained from a pregnant woman at 5^{th} to 15^{th} week of gestation.

According to still further features in the described preferred embodiments the immunologically staining is effected using an antibody directed against a trophoblast specific antigen.

According to still further features in the described preferred embodiments the trophoblast specific antigen is selected from the group consisting of HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

According to still further features in the described preferred embodiments the in situ chromosomal and/or DNA analysis is effected using fluorescent in situ hybridization (FISH), primed in situ labeling (PRINS), multicolor-banding (MCB) and/or quantitative FISH (Q-FISH).

According to still further features in the described preferred embodiments the Q-FISH is effected using a peptide nucleic acid (PNA) oligonucleotide probe.

According to still further features in the described preferred embodiments the at least one chromosomal abnormality is selected from the group consisting of aneuploidy, translocation, subtelomeric rearrangement, unbalanced subtelomeric rearrangement, deletion, microdeletion, inversion, duplication, and telomere instability and/or shortening.

According to still further features in the described preferred embodiments the chromosomal aneuploidy is a complete and/or partial trisomy.

According to still further features in the described preferred embodiments the trisomy is selected from the group consisting of trisomy 21, trisomy 18, trisomy 13, trisomy 16, XXY, XYY, and XXX.

According to still further features in the described preferred embodiments the chromosomal aneuploidy is a complete and/or partial monosomy.

According to still further features in the described preferred embodiments the monosomy is selected from the group consisting of monosomy X, monosomy 21, monosomy 22, monosomy 16 and monosomy 15.

According to still further features in the described preferred embodiments the method further comprising a step of isolating the at least one stained trophoblast cell prior to step (c).

According to still further features in the described preferred embodiments isolating the at least one stained trophoblast is effected using laser microdissection.

According to still further features in the described preferred embodiments the genetic analysis utilizes at least one method selected from the group consisting of comparative genome hybridization (CGH) and identification of at least one nucleic acid substitution.

According to still further features in the described preferred embodiments the CGH is effected using metaphase chromosomes and/or a CGH-array.

According to still further features in the described preferred embodiments the identification of at least one nucleic acid substitution is effected using a method selected from the group consisting of DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

According to still further features in the described preferred embodiments the at least one DNA abnormality is selected from the group consisting of single nucleotide substitution, micro-deletion, micro-insertion, short deletions, short insertions, multinucleotide changes, DNA methylation and loss of imprint (LOI).

According to still further features in the described preferred embodiments the at least one chromosomal abnormality is selected from the group consisting of aneuploidy, deletion, microdeletion, duplication, unbalanced translocation, unbalanced inversion, unbalanced chromosomal rearrangement, and unbalanced subtelomeric rearrangement.

According to still further features in the described preferred embodiments the RNA-ISH staining is effected using a probe selected from the group consisting of an RNA molecule, a DNA molecule and a PNA oligonucleotide.

According to still further features in the described preferred embodiments the RNA molecule is an RNA oligonucleotide and/or an in vitro transcribed RNA.

According to still further features in the described preferred embodiments the DNA molecule is an oligonucleotide and/or a cDNA molecule.

According to still further features in the described preferred embodiments the probe is selected capable of identifying a trophoblast specific RNA transcript.

According to still further features in the described preferred embodiments the trophoblast specific RNA transcript is selected from the group consisting of H19, HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

According to still further features in the described preferred embodiments the genetic analysis utilizes a method selected from the group consisting of PCR, and/or PCR-RFLP.

According to still further features in the described preferred embodiments the genetic analysis is capable of detecting short tandem repeats, variable number of tandem repeats (VNTR) and/or minisatellites variant repeats (MVR).

According to still further features in the described preferred embodiments the prestained specimen is subjected to immunological staining.

According to still further features in the described preferred embodiments the immunological staining is effected using an antibody directly or indirectly conjugated to an enzyme.

According to still further features in the described preferred embodiments the enzyme is selected from the group consisting of HRP and alkaline phosphatase.

According to still further features in the described preferred embodiments the HRP hydrolyses a chromogenic substrate selected from the group consisting of Nova Red, diaminobenzidine (DAB), Vector(R) SG substrate, and luminol-based chemiluminescent substrate.

According to still further features in the described preferred embodiments the alkaline phosphatase hydrolyses a chromogenic substrate selected from the group consisting of AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate.

According to still further features in the described preferred embodiments the prestained specimen is subjected to activity staining.

According to still further features in the described preferred embodiments the activity staining is effected using a chromogenic substrate.

According to still further features in the described preferred embodiments the chromogenic substrate is selected from the group consisting of Nova Red, diaminobenzidine (DAB), Vector(R) SG substrate, luminol-based chemiluminescent substrate, AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate.

According to still further features in the described preferred embodiments the prestained specimen is subjected to cytological staining.

According to still further features in the described preferred embodiments the cytological staining is effected using a staining selected from the group consisting of May-Grünwald-Giemsa, Giemsa, Papanicolau, Hematoxylin, and Hematoxylin-Eosin.

According to still further features in the described preferred embodiments incubating the prestained specimen of cells or tissue in the solution containing ammonium hydroxide is effected for at least 2 seconds.

According to still further features in the described preferred embodiments incubating the prestained specimen of cells or tissue in the solution containing ammonium hydroxide is effected for at least 1 minute.

According to still further features in the described preferred embodiments the incubating the prestained specimen of cells or tissue in the solution containing ammonium hydroxide is effected for a time period selected from the range of 1 second to 180 minutes.

According to still further features in the described preferred embodiments incubating the prestained specimen of cells or tissue in the solution containing ammonium hydroxide is effected for a time period selected from the range of 1 minute to 60 minutes.

According to still further features in the described preferred embodiments the incubating the prestained specimen of cells or tissue in the solution containing ammonium hydroxide is effected for 45 minutes.

According to still further features in the described preferred embodiments the ammonium hydroxide is provided at a concentration selected from the range of 0.1 to 28%.

According to still further features in the described preferred embodiments the ammonium hydroxide is provided at a concentration selected from the range of 0.1% to 5%.

According to still further features in the described preferred embodiments the ammonium hydroxide is provided at a concentration of 2%.

According to still further features in the described preferred embodiments the method of identifying an embryonic cell in a mixed cell population, further comprising evaluating at least one morphological criterion selected from the group consisting of nuclei multiplicity, nuclei arrangement, nucleus shape, cytoplasm condensation, cytoplasm shape, and cytoplasm/nucleus orientation.

According to still further features in the described preferred embodiments the embryonic cell is extravillous trophoblast type I and whereas the nucleus shape is egg-shape, round or amorphy, the cell exhibits a variably condensed chromatin, the cytoplasm condensation is homogenously condensed and the cytoplasm/nucleus orientation is such that the cytoplasm encompasses 50-85% of the nucleus, thereby identifying the embryonic cells in the mixed cell population.

According to still further features in the described preferred embodiments the embryonic cell is extravillous trophoblast type II and whereas the nucleus shape is egg-shape, round, or amorphy, the nucleus multiplicity is two nuclei, the cell exhibits a homogenously condensed chromatin, the nucleus/cytoplasm ratio is at least 2:1, the cytoplasm condensation is homogenously condensed, and the cytoplasm/nucleus orientation is such that the cytoplasm encompasses 50-85% of the nucleus, thereby identifying the embryonic cells in the mixed cell population.

According to still further features in the described preferred embodiments the embryonic cell is extravillous trophoblast type III and whereas the nucleus shape is round or egg-shape, the cell exhibits a homogenously condensed chromatin, the nucleus/cytoplasm ratio is at least 2:1, respectively, the cytoplasm condensation is homogenously condensed, and the cytoplasm/nucleus orientation is such that the cytoplasm is basal to the nucleus and encompasses about 50% of the nucleus, thereby identifying the embryonic cells in the mixed cell population.

According to still further features in the described preferred embodiments the embryonic cell is extravillous trophoblast type IV and whereas the nucleus shape is horseshoe-shape, the cell exhibits a homogenously condensed chromatin, the cytoplasm shape is fluffy, the nucleus/cytoplasm ratio is about 1:1, the cytoplasm condensation is homogenously condensed, and the cytoplasm/nucleus orientation is such that the cytoplasm encompasses 50-80% of the nucleus, thereby identifying the embryonic cells in the mixed cell population.

According to still further features in the described preferred embodiments the embryonic cell is extravillous trophoblast type V and whereas the nucleus multiplicity is two nuclei, the cell exhibits a homogenously condensed chromatin, the nucleus/cytoplasm ratio is about 1:1, the cytoplasm condensation is variably condensed, and the cytoplasm/nucleus orientation is such that the cytoplasm encompasses 70-100% of the two nuclei, thereby identifying the embryonic cells in the mixed cell population.

According to still further features in the described preferred embodiments the embryonic cell is extravillous trophoblast clump type I and whereas the nucleus multiplicity is more than two, nucleus shape is round, egg-shape or amorphy, the cell exhibits a variably condensed chromatin, the nuclei arrangement is random, the cytoplasm shape is fluffy, the cytoplasm condensation is homogenously condensed, thereby identifying the embryonic cells in the mixed cell population.

According to still further features in the described preferred embodiments the embryonic cell is extravillous trophoblast clump type II and whereas the nucleus multiplicity is more than two, nucleus shape is variable, the cell exhibits a homogenously condensed chromatin, the cytoplasm shape is fluffy, the cytoplasm condensation is variable, the nuclei arrangement is in a row, thereby identifying the embryonic cells in the mixed cell population.

According to still further features in the described preferred embodiments the embryonic cell is syncytiotrophoblast and whereas the nucleus multiplicity is more than 10, nucleus shape is variable, the cell exhibits a homogenously condensed chromatin, the cytoplasm shape is fluffy with a well-defined cytoplasm border, the cytoplasm condensation is variable, the nuclei arrangement is random, and the cytoplasm/nucleus orientation is such that the cytoplasm is common to the nuclei, thereby identifying the embryonic cells in the mixed cell population.

According to still further features in the described preferred embodiments evaluating the at least one morphological criterion is effected by staining.

According to still further features in the described preferred embodiments the staining is selected from the group consisting of a cytological staining, an activity staining and/or an immunological staining.

According to still further features in the described preferred embodiments the immunological staining is effected using an antibody directed against a trophoblast specific antigen.

The present invention successfully addresses the shortcomings of the presently known configurations by providing a non-invasive, risk-free method of prenatal diagnosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-d are photomicrographs illustrating IHC (FIGS. 1a, c) and FISH (FIGS. 1b, d) analyses of transcervical cells. Transcervical cells obtained from two pregnant women at the 7th (FIGS. 1a-b, case 73 in Table 1) and the 9th (FIGS. 1c-d, case 80 in Table 1) week of gestation were subjected to IHC using the HLA-G antibody (mAb 7759, Abcam) followed by FISH analysis using the CEP X green and Y orange (Abbott, Cat. 5J10-51) probes. Shown are HLA-G-positive extravillous trophoblast cells with a reddish cytoplasm (FIG. 1a, a cell marked with a black arrow; FIG. 1c, two cells before cell division marked with two black arrows). Note the single orange and green signals in each trophoblast cell (FIGS. 1b, and d, white arrows), corresponding to the Y and X chromosomes, respectively, demonstrating the presence of a normal male fetus in each case.

FIGS. 2a-b are photomicrographs illustrating IHC (FIG. 2a) and FISH (FIG. 2b) analyses of transcervical cells. Transcervical cells obtained from a pregnant women at the 11th (FIGS. 2a-b, case 223 in Table 1) week of gestation were subjected to IHC using the PLAP antibody (Zymed, Cat. No. 18-0099) followed by FISH analysis using the CEP X green and Y orange (Abbott, Cat. 5J10-51) probes. Shown is a PLAP-positive villous cytotrophoblast cell with a reddish cytoplasm (FIG. 2a, black arrow). Note the single orange and green signals in the villous cytotrophoblast cell (FIG. 2b, white arrows), corresponding to the Y and X chromosomes, respectively, demonstrating the presence of a normal male fetus.

FIGS. 3a-b are photomicrographs illustrating IHC (FIG. 3a) and FISH (FIG. 3b) analyses of transcervical cells. Transcervical cells obtained from a pregnant woman at the 8th week of gestation (case 71 in Table 1) were subjected to IHC using the HLA-G antibody (mAb 7759, Abcam) followed by FISH analysis using the LSI 21q22 orange and the CEP Y green (Abbott, Cat. No. # 5J10-24 and 5J13-02) probes. Note the reddish cytoplasm of the trophoblast cell following HLA-G antibody reaction (FIG. 3a, white arrow) and the presence of three orange and one green signals corresponding to chromosomes 21 and Y, respectively, (FIG. 3b, white arrows), demonstrating the presence of trisomy 21 in a male fetus.

FIGS. 4a-b are photomicrographs illustrating IHC (FIG. 4a) and FISH (FIG. 4b) analyses of transcervical cells. Transcervical cells obtained from a pregnant woman at the 6th week of gestation (case 76 in Table 1) were subjected to IHC using the HLA-G antibody followed by FISH analysis using the CEP X green and Y orange (ABBOTT, Cat. # 5J10-51) probes. Note the reddish color in the cytoplasm of the trophoblast cell following HLA-G antibody reaction (FIG. 4a, black arrow) and the single green signal corresponding to a single X chromosome (FIG. 4b, white arrow) demonstrating the presence of a female fetus with Turner's syndrome.

FIGS. 5a-c are photomicrographs illustrating IHC (FIG. 5a) and FISH (FIGS. 5b, c) analyses of transcervical (FIGS. 5a-b) or placental (FIG. 5c) cells obtained from a pregnant woman at the 7th week of gestation (case 161 in Table 1). FIGS. 5a-b—Transcervical cells were subjected to IHC using the HLA-G antibody (mAb 7759, Abcam) and FISH analysis using the CEP X green and Y orange (Abbott, Cat. # 5J10-51) probes. Note the reddish color in the cytoplasm of two trophoblast cells (FIG. 5a, cells Nos. 1 and 2) and the presence of two green signals and a single orange signal corresponding to two X and a single Y chromosomes in one trophoblast cell (FIG. 5b, cell No. 1) and the presence of a single green and a single orange signals corresponding to a single X and a single Y chromosomes in a second trophoblast cell (FIG. 5b, cell No. 2), indicating mosaicism for Klinefelter's syndrome in the trophoblast cells. FIG. 5c-Placental cells were subjected to FISH analysis using the CEP X green and Y orange (Abbott, Cat. # 5J10-51) probes. Note the presence of a single green and a single orange signals corresponding to a single X and a single Y chromosomes in one placental cell (FIG. 5c, cell No. 1) and the presence of two green signals and a single orange signal corresponding to two X and a single Y chromosomes in the second placental cell (FIG. 5c, cell No. 2), indicating mosaicism for Klinefelter's syndrome in the placental cells.

FIGS. 6a-b are photomicrographs illustrating IHC (FIG. 6a) and FISH (FIG. 6b) analyses of transcervical cells obtained from a pregnant woman at the 7^{th} week of gestation. Immunohistochemistry was performed using the mouse anti human trophoblast protein NDOG-1 (MCA277, Serotec immunological excellence, UK) at a 1:50 dilution and the aminoethylcarbazole (AEC) detection method, followed by a FISH analysis using the CEP X and Y probes. Note the specific label of the syncytiotrophoblast in the transcervical specimen using the NDOG-1 antibody (FIG. 6a) and the subsequent FISH signals confirming the presence of X and Y chromosomes in the synctiotrophoblast fetal cells.

FIGS. 7a-d are photomicrographs illustrating IHC of extravillous trophoblast cell clump (Clump Type I) present in a transcervical specimen. IHC was performed using the HLA-G antibody and the HRP-AEC detection method (brown staining, mostly in the cytoplasm), followed by counterstaining in the presence of 2% Hematoxylin (deep purple staining of the nuclei and light purple or reddish staining of the cytoplasm). Note the presence of multiple nuclei in various sizes, shape and/or condensity and the amorphic and condensed reddish cytoplasm.

FIGS. 8a-b are photomicrographs illustrating IHC of extravillous trophoblast cell clumps (Clump Type II) present in a transcervical specimen. IHC was performed using the HLA-G antibody and the HRP-AEC detection method (brown staining, mostly in the cytoplasm), followed by counterstaining in the presence of 2% Hematoxylin (deep purple staining of the nuclei and light purple or reddish staining of the cytoplasm). Note the formation of rows within the extravillous trophoblast cell clumps (Clump Type II), and the homogenous and condensed (deep purple staining) nuclei.

FIGS. 9a-b are photomicrographs illustrating IHC of syncytiotrophoblast present in a transcervical specimen. IHC was performed using the NDOG-1 antibody and the HRP-AEC detection method (brown staining, mostly in the cytoplasm), followed by counterstaining in the presence of 2% Hematoxylin (deep purple staining of the nuclei and light purple staining of the cytoplasm). Note the presence of multiple nuclei with common, well-defined and fluffy cytoplasm of the entire syncitium; also note that while each of the nuclei in each syncitium exhibits homogenous size and shape, the nuclei condensity varies between different syncitium.

FIGS. 10a-c are photomicrographs illustrating IHC of extravillous trophoblast Type I present in a transcervical specimen. IHC was performed using the HLA-G antibody and the HRP-AEC detection method (brown staining, mostly in the cytoplasm), followed by counterstaining in the presence of 2% Hematoxylin (deep purple staining of the nuclei and deep staining of the cytoplasm). Note the egg-shape nucleus, with variable degrees of condensation and the condensed and homogenous cytoplasm (deep red) which surrounds or covers some of the nucleus of the extravillous trophoblast (Type I). Also note the 3 to 1 ratio between the nucleus and the cytoplasm, respectively.

FIGS. 11a-b are photomicrographs illustrating IHC of extravillous trophoblasts Type II present in a transcervical specimen. IHC was performed using the HLA-G antibody and the HRP-AEC detection method (brown staining, mostly in the cytoplasm), followed by counterstaining in the presence of 2% Hematoxylin (deep purple staining of the nuclei and deep staining of the cytoplasm). Note the egg-shape to square shape nucleus, with uniform size and degree of condensation of the extravillous trophoblast (Type II). Also note the 4 to 1 ratio between the nucleus and the cytoplasm, respectively.

FIGS. 12a-b are photomicrographs illustrating IHC of extravillous trophoblast Type III present in a transcervical specimen. IHC was performed using the HLA-G antibody and the HRP-AEC detection method (brown staining, mostly in the cytoplasm), followed by counterstaining in the presence of 2% Hematoxylin (deep purple staining of the nuclei and deep staining of the cytoplasm). Note the large (about ⅔ of the cell radius) and condensed nucleus, located basal to the cytoplasm of the extravillous trophoblast (Type III). Also note the deep red staining of the condensed and homogenous cytoplasm, which surrounds only part of the nucleus.

FIG. 13 is a photomicrograph illustrating IHC of an extravillous trophoblast Type IV present in a transcervical specimen. IHC was performed using the HLA-G antibody and the HRP-AEC detection method (brown staining, mostly in the cytoplasm), followed by counterstaining in the presence of 2% Hematoxylin (deep purple staining of the nuclei and deep staining of the cytoplasm). Note the large (about ⅔ of the cell radius) and condensed nucleus exhibiting a horseshoe shape (not oval, not round) of the extravillous trophoblast (Type IV). Also note the homogenous and fluffy cytoplasm, with no definite borders.

FIG. 14 is a photomicrograph illustrating IHC of an extravillous trophoblast Type V present in a transcervical specimen. IHC was performed using the HLA-G antibody and the HRP-AEC detection method (brown staining, mostly in the cytoplasm), followed by counterstaining in the presence of 2% Hematoxylin (deep purple staining of the nuclei and deep staining of the cytoplasm). Note the dividing nucleus and the large and variably condensed cytoplasm (deep purple) which surrounds the entire nucleus of the extravillous trophoblast (Type V).

FIGS. 15a-d are photomicrographs illustrating IHC of an epithelial cell present in a transcervical specimen. IHC was performed using the HLA-G antibody and the HRP-AEC detection method (brown staining in the cytoplasm), followed by counterstaining in the presence of 2% Hematoxylin (purple staining of the nuclei). Note the typically small nucleus characteristics of an epithelial cell.

FIGS. 16a-b are photomicrographs illustrating IHC of non-fetal cells (false cell type I) present in a transcervical specimen which are non-specifically stained with the HLA-G antibody. IHC was performed using the HLA-G antibody and the HRP-AEC detection method (brown staining in the cytoplasm), followed by counterstaining in the presence of 2% Hematoxylin (purple staining of the nuclei). Note the small (about ⅕ of the cell radius), round shape nucleus with variable condensity and the large, amorphic cytoplasm, with various red color.

FIGS. 17a-c are photomicrographs illustrating IHC of non-fetal cells (false cell type II) present in a transcervical specimen which are non-specifically stained with the HLA-G antibody. IHC was performed using the HLA-G antibody and the HRP-AEC detection method (brown staining in the cytoplasm), followed by counterstaining in the presence of 2% Hematoxylin (purple staining of the nuclei). Note the small, round/oval nucleus with variable condensity and the common cytoplasm that surrounds half or less of nucleus borders. In no parts cytoplasm climbs over the nuclei.

FIGS. 18a-b are photomicrographs illustrating IHC of non-fetal cells (false cell type III) present in a transcervical specimen which are non-specifically stained with the HLA-G antibody. IHC was performed using the HLA-G antibody and the HRP-AEC detection method (brown staining in the cytoplasm), followed by counterstaining in the presence of 2% Hematoxylin (purple staining of the nuclei). Note the one or two egg shaped nucleus, with variable condensation and the amorphic cytoplasm with no definitive borders colored deep red. The cytoplasm surrounds some of the nucleus, and in some parts climbs over it.

FIGS. 19a-f are photomicrographs illustrating IHC of non-fetal cells (false cell type IV) present in a transcervical specimen which are non-specifically stained with the HLA-G antibody. IHC was performed using the HLA-G antibody and the HRP-AEC detection method (brown staining in the cytoplasm), followed by counterstaining in the presence of 2% Hematoxylin (purple staining of the nuclei). Note very small, round shaped and condensed nucleus with a very small cytoplasm (respectively to nucleus), colored deep red cytoplasm which surrounds either most or all of nucleus.

FIGS. 20a-b are photomicrographs illustrating IHC of false cell artifacts present in a transcervical specimen which are non-specifically stained with the HLA-G antibody. IHC was performed using the HLA-G antibody and the HRP-AEC detection method (brown staining in the cytoplasm), followed by counterstaining in the presence of 2% Hematoxylin (purple staining of the nuclei). Note the absence of nucleus and the various amorphous structures resembling a cytoplasm in a very deep red color.

### DESCRIPTION OF THE PREFERRED EMBODIMENTS

The present invention is of a method of identifying at least one chromosomal abnormality in a fetus and of determining fetal gender. Specifically, the present invention provides a non-invasive, risk-free prenatal diagnosis method which can be used to determine genetic abnormalities such as chromosomal anueploidy, translocations, inversions, deletions and microdeletions present in a fetus.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Early detection of fetal abnormalities and prenatal diagnosis of genetic abnormalities is crucial for carriers of genetic diseases such as, common translocations (e.g., Robertsonian translocation), chromosomal deletions and/or microdeletions (e.g., Angelman syndrome, DiGeorge syndrome) as well as for couples with advanced maternal age (e.g., over 35 years) which are subjected to increased risk for a variety of chromosomal anueploidy (e.g., Down syndrome).

Current methods of prenatal diagnosis include cytogenetic and FISH analyses which are performed on fetal cells obtained via amniocentesis or chorionic villi sampling (CVS). However, although efficient in predicting chromosomal aberrations, the amniocentesis or CVS procedures carry a 0.5-1% or 2-4% of procedure related risks for miscarriage, respectively. Because of the relatively high risk of miscarriage, amniocentesis or CVS is not offered to women under the age of 35 years. Thus, as a result of not being tested, the vast majority (80%) of Down syndrome babies are actually born to women under 35 years of age. Therefore, it is important to develop methods for non-invasive, risk-free prenatal diagnosis which can be offered to all women, at any maternal age.

The discovery of fetal nucleated erythrocytes in the maternal blood early in gestation have prompted many investigators to develop methods of isolating these cells and subjecting them to genetic analysis (e.g., PCR, FISH). However, since the frequency of nucleated fetal cells in the maternal blood is exceptionally low (0.0035%), the NRBC cells had to be first purified (e.g., using Ficol-Paque or Percoll-gradient density centrifugation) and then enriched using for example, magnetic activated cell sorting (MACS, Busch, J. et al., 1994, Prenat. Diagn. 14: 1129-1140), ferrofluid suspension (Steele, C. D. et al., 1996, Clin. Obstet. Gynecol. 39: 801-813), charge flow separation (Wachtel, S. S. et al., 1996, Hum. Genet. 98:162-166), or FACS analysis (Wang, J. Y. et al., 2000, Cytometry 39:224-230).

U.S. Pat. No. 5,750,339 discloses genetic analysis of fetal cells derived from the maternal. In order to traverse the limitations described above, the fetal cells of the sample are enriched using antiCD71, CD36 and/or glycophorin A and the maternal cells are depleted using anti-maternal antibodies such as anti-CD14, CD4, CD8, CD3, CD19, CD32, CD16 and CD4. Resultant fetal cells are identified using an HLA-G specific probe. Although recovery of fetal NRBCs can be effected using such an approach, inconsistent recovery rates coupled with limited sensitivity prevents clinical application of diagnostic techniques using fetal NRBCs (Bischoff, F. Z. et al., 2002. Hum. Repr. Update 8: 493-500).

Another fetal cell type which has been identified as a potential target for diagnosis is the trophoblast. Prior art studies describe the identification of trophoblast cells in transcervical specimens using a variety of antibodies such as HLA-G (Bulmer, J. N. et al., 2003. Prenat. Diagn. 23: 34-39), PLAP, FT1.41.1, NDOG-1, NDOG-5, and 340 (Miller et al., 1999. Human Reproduction, 14: 521-531). In these studies the antibodies recognized trophoblasts cells in 30-79% of the transcervical specimens. In addition, the FISH, PCR and/or quantitative fluorescent PCR (QF-PCR) analyses, which were performed on duplicated transcervical specimens, were capable of identifying approximately 80-90% of all male fetuses. However, since the DNA (e.g., FISH and/or PCR) and immunological (e.g., IHC) analyses were performed on separated slides, these methods were impractical for diagnosing fetal chromosomal abnormalities.

While reducing the present invention to practice and experimenting with approaches for improving genetic diagnosis of fetuses, the present inventors have devised a non-invasive, risk-free method of determining fetal gender and/or identifying chromosomal abnormality of a fetus.

As described hereinunder and in Examples 1 and 2 of the Examples section which follows, the present inventors have devised a method of sequentially staining transcervical cells with a trophoblast specific antibody [e.g., directed against HLA-G, PLAP and/or MCAM (CHL1)] followed by FISH analysis of stained cells. As is shown in Table 3 and in Examples 1 and 2 of the Examples section which follows, using the method of the present invention a correct determination of fetal chromosomal FISH pattern was achieved in 92.45% of trophoblast-containing transcervical specimens obtained from ongoing pregnancies and/or prior to pregnancy termination, thereby, conclusively showing that the present method is substantially more accurate than prior art approaches in diagnosis of fetus genetic abnormalities.

Thus, according to one aspect of the present invention there is provided a method of determining fetal gender and/or identifying at least one chromosomal abnormality of a fetus. The term “fetus” as used herein refers to an unborn human offspring (i.e. an embryo and/or a fetus) at any embryonic stage.

As used herein “fetal gender” refers to the presence or absence of the X and/or Y chromosome(s) in the fetus.

As used herein “chromosomal abnormality” refers to an abnormal number of chromosomes (e.g., trisomy 21, monosomy X) or to chromosomal structure abnormalities (e.g., deletions, translocations, etc).

According to the present method, identification of fetus gender and/or at least one chromosomal abnormality is effected by first immunologically staining a trophoblast-containing cell sample to thereby identify at least one trophoblast cell, and subsequently subjecting the trophoblast cell(s) identified to in situ chromosomal and/or DNA analysis to thereby determine fetal gender and/or identify at least one chromosomal abnormality.

The term “trophoblast” refers to an epithelial cell which is derived from the placenta of a mammalian embryo or fetus; trophoblast typically contact the uterine wall. There are three types of trophoblast cells in the placental tissue: the villous cytotrophoblast, the syncytiotrophoblast, and the extravillous trophoblast, and as such, the term “trophoblast” as used herein encompasses any of these cells. The villous cytotrophoblast cells are specialized placental epithelial cells which differentiate, proliferate and invade the uterine wall to form the villi. Cytotrophoblasts, which are present in anchoring villi can fuse to form the syncytiotrophoblast layer or form columns of extravillous trophoblasts (Cohen S. et al., 2003. J. Pathol. 200: 47-52).

A trophoblast-containing cell sample can be any biological sample which includes trophoblasts, whether viable or not. Preferably, a trophoblast-containing cell sample is a blood sample or a transcervical and/or intrauterine sample derived from a pregnant woman at various stages of gestation.

Presently preferred trophoblast samples are those obtained from a cervix and/or a uterine of a pregnant woman (transcervical and intrauterine samples, respectively).

The trophoblast containing cell sample utilized by the method of the present invention can be obtained using any one of numerous well known cell collection techniques.

According to preferred embodiments of the present invention the trophoblast-containing cell sample is obtained using mucus aspiration (Sherlock, J., et al., 1997. J. Med. Genet. 34: 302-305; Miller, D. and Briggs, J. 1996. Early Human Development 47: S99-S102), cytobrush (Cioni, R., et al., 2003. Prent. Diagn. 23: 168-171; Fejgin, M. D., et al., 2001. Prenat. Diagn. 21: 619-621), cotton wool swab (Griffith-Jones, M. D., et al., 1992. Supra), endocervical lavage (Massari, A., et al., 1996. Hum. Genet. 97: 150-155; Griffith-Jones, M. D., et al., 1992. Supra; Schueler, P. A. et al., 2001. 22: 688-701), and intrauterine lavage (Cioni, R., et al., 2002. Prent. Diagn. 22: 52-55; Ishai, D., et al., 1995. Prenat. Diagn. 15: 961-965; Chang, S-D., et al., 1997. Prenat. Diagn. 17: 1019-1025; Sherlock, J., et al., 1997, Supra; Bussani, C., et al., 2002. Prenat. Diagn. 22: 1098-1101). See for comparison of the various approaches Adinolfi, M. and Sherlock, J. (Human Reprod. Update 1997, 3: 383-392 and J. Hum. Genet. 2001, 46: 99-104), Rodeck, C., et al. (Prenat. Diagn. 1995, 15: 933-942). The cytobrush method is the presently preferred method of obtaining the trophoblast-containing cell sample of the present invention.

In the cytobrush method, a Pap smear cytobrush (e.g., MedScand-AB, Malmö, Sweden) is inserted through the external os to a maximum depth of 2 cm and removed while rotating it a full turn (i.e., 360°). In order to remove the transcervical cells caught on the brush, the brush is shaken into a test tube containing 2-3 ml of a tissue culture medium (e.g., RPMI-1640 medium, available from Beth Haemek, Israel) in the presence of 1% Penicillin Streptomycin antibiotic. In order to concentrate the transcervical cells on microscopic slides cytospin slides are prepared using e.g., a Cytofunnel Chamber Cytocentrifuge (Thermo-Shandon, England). It will be appreciated that the conditions used for cytocentrifugation are dependent on the murkiness of the transcervical specimen; if the specimen contained only a few cells, the cells are first centrifuged for 5 minutes and then suspended with 1 ml of fresh medium. Once prepared, the cytospin slides can be kept in 95% alcohol until further use.

As is shown in Table 3 and in Examples 1 and 2 of the Examples section which follows, using the cytobrush method, the present inventors obtained trophoblast-containing cell samples in 348 out of the 396 transcervical specimens collected.

Since trophoblast cells are shed from the placenta into the uterine cavity, the trophoblast-containing cell samples should be retrieved as long as the uterine cavity persists, which is until about the 13-15 weeks of gestation (reviewed in Adinolfi, M. and Sherlock, J. 2001, Supra).

Thus, according to preferred embodiments of the present invention the trophoblast-containing cell sample is obtained from a pregnant woman at 5^{th} to 15^{th} week of gestation. Preferably, the cells are obtained from a pregnant woman between the 5^{th} to 13^{th} week of gestation, more preferably, between the 5^{th} to the 11^{th} week of gestation, even more preferably between the 5^{th} to the 8^{th} week of gestation.

It will be appreciated that the determination of the exact week of gestation during a pregnancy is well within the capabilities of one of ordinary skill in the art of Gynecology and Obstetrics.

Once obtained, the trophoblast-containing cell sample (e.g., the cytospin preparation thereof) is subjected to an immunological staining.

According to preferred embodiments of the present invention, immunological staining is effected using an antibody directed against a trophoblast specific antigen.

Antibodies directed against trophoblast specific antigens are known in the arts and include, for example, the HLA-G antibody, which is directed against part of the non-classical class I major histocompatibility complex (MHC) antigen specific to extravillous trophoblast cells (Loke, Y. W. et al., 1997. Tissue Antigens 50: 135-146), the anti human placental alkaline phosphatase (PLAP) antibody which is specific to the syncytiotrophoblast and/or cytotrophoblast (Leitner, K. et al., 2001, J. Histochemistry and Cytochemistry, 49: 1155-1164), the CHL1 (CD146) antibody which is directed against the melanoma cell adhesion molecule (MCAM) (Higuchi T., et al., 2003, Mol. Hum. Reprod. 9: 359-366), the CHL2 antibody which is directed against laeverin, a novel protein that belongs to membrane-bound gluzincin metallopeptidases and expressed on trophoblasts (Fujiwara H., et al., 2004, Biochem. Biophys. Res. 313: 962-968), the H315 antibody which interacts with a human trophoblast membrane glycoprotein present on the surface of fetal cells (Covone A E and Johnson P M, 1986, Hum. Genet. 72: 172-173), the FT1.41.1 antibody which is specific for syncytiotrophoblasts and the 103 antibody (Rodeck, C., et al., 1995. Prenat. Diag. 15: 933-942), the NDOG-1 antibody which is specific for syncytiotrophoblasts (Miller D., et al. Human Reproduction, 1999, 14: 521-531), the NDOG-5 antibody which is specific for extravillous cytotrophoblasts (Miller D., et al. 1999, Supra), the BC1 antibody (Bulmer, J. N. et al., Prenat. Diagn. 1995, 15: 1143-1153), the AB-154 or AB-340 antibodies which are specific to syncytio—and cytotrophoblasts or syncytiotrophoblasts, respectively (Durrant L et al., 1994, Prenat. Diagn. 14: 131-140), the protease activated receptor (PAR)-1 antibody which is specific for placental cells during the 7th and the 10th week of gestation (Cohen S. et al., 2003. J. Pathol. 200: 47-52), the glucose transporter protein (Glut)-12 antibody which is specific to syncytiotrophoblasts and extravillous trophoblasts during the 10th and 12th week of gestation (Gude N M et al., 2003. Placenta 24:566-570), the anti factor XIII antibody which is specific to the cytotrophoblastic shell (Asahina, T., et al., 2000. Placenta, 21: 388-393; Kappelmayer, J., et al., 1994. Placenta, 15: 613-623), the Mab FDO202N directed against the human placental lactogen hormone (hPLH) which is expressed by extravillous trophoblasts (Latham S E, et al., Prenat Diagn. 1996; 16(9):813-21).

It will be appreciated that antibodies against other proteins which are expressed on trophoblast cells can also be used along with the present invention. Examples include, but are not limited to, the HLA-C which is expressed on the surface of normal trophoblast cells (King A, et al., 2000, Placenta 21: 376-87; Hammer A, et al., 1997, Am. J. Reprod. Immunol. 37: 161-71), the JunD and Fra2 proteins (members of the API transcription factor) which are expressed on extravillous trophoblasts (Bamberger A M, et al., 2004, Mol. Hum. Reprod. 10: 223-8), the nucleoside diphosphate kinase A (NDPK-A) protein which is encoded by the nm23-H1 gene and is expressed in extravillous trophoblasts during the first trimester (Okamoto T, et al., 2002, Arch. Gynecol. Obstet. 266: 1-4), Tapasin (Copeman J, et al., 2000, Biol. Reprod. 62: 1543-50), the CAR protein (coxsackie virus and adenovirus receptor) which is expressed in invasive or extravillous trophoblasts but not in villous trophoblasts (Koi H, et al., 2001, Biol. Reprod. 64: 1001-9), the human Achaete Scute Homologue-2 (HASH2) protein which is expressed in extravillous trophoblasts (Alders M, et al., 1997, Hum. Mol. Genet. 6: 859-67; Guillemot F, et al., 1995, Nat. Genet. 9: 235-42), the human chorion gonadotropin alpha (αHCG) which is expressed in trophoblasts (Schueler P A, et al., 2001, Placenta 22: 702-15), the insulin-like growth factor-II (IGF-II), the plasminogen activator inhibitor-1 (PAI-1; Li F et al., Exp Cell Res. 2000, 258: 245-53), p57(KIP2) which is expressed in trophoblasts (Tsugu A et al., Am J Pathol. 2000; 157: 919-32), the placental protein 5 (PP5) which is identical to tissue factor pathway inhibitor-2 (TFPI-2) and is expressed by cytotrophoblasts (Hube F et al., Biol Reprod. 2003; 68: 1888-94) and the placenta-specific genes (PLAC1, PLAC8 and PLAC9) which are exclusively expressed by cells of the trophoblastic lineage (Fant M et al., Mol Reprod Dev. 2002; 63: 430-6; Galaviz-Hernandez C, et al., 2003, Gene 309: 81-9; Cocchia M, et al., 2000, Genomics 68: 305-12).

Immunological staining is based on the binding of labeled antibodies to antigens present on the cells. Examples of immunological staining procedures include but are not limited to, fluorescently labeled immunohistochemistry (using a fluorescent dye conjugated to an antibody), radiolabeled immunohistochemistry (using radiolabeled e.g., ¹²⁵I, antibodies) and immunocytochemistry [using an enzyme (e.g., horseradish peroxidase) and a chromogenic substrate]. Preferably, the immunological staining used by the present invention is immunohistochemistry and/or immunocytochemistry.

Immunological staining is preferably followed by counterstaining the cells using a dye which binds to non-stained cell compartments. For example, if the labeled antibody binds to antigens present on the cell cytoplasm, a nuclear stain (e.g., Hematoxylin-Eosin stain) is an appropriate counterstaining.

Methods of employing immunological stains on cells are known in the art. Briefly, to detect a trophoblast cell in a transcervical specimen, cytospin slides are washed in 70% alcohol solution and dipped for 5 minutes in distilled water. The slides are then transferred into a moist chamber, washed three times with phosphate buffered-saline (PBS). To visualize the position of the transcervical cells on the microscopic slides, the borders of the transcervical specimens are marked using e.g., a Pap Pen (Zymed Laboratories Inc., San Francisco, Calif., USA). To block endogenous cell peroxidase activity 50 μl of a 3% hydrogen peroxide (Merck, Germany) solution are added to each slide for a 10-minute incubation at room temperature following which the slides are washed three times in PBS. To avoid non-specific binding of the antibody, two drops of a blocking reagent (e.g., Zymed HISTOSTAIN®-PLUS Kit, Cat No. 858943) are added to each slide for a 10-minute incubation in a moist chamber. To identify the fetal trophoblast cells in the transcervical sample, an aliquot (e.g., 50 μl) of a trophoblast-specific antibody [e.g., anti HLA-G antibody (mAb 7759, Abcam Ltd., Cambridge, UK) or anti human placental alkaline phosphatase antibody (PLAP, Cat. No. 18-0099, Zymed)] is added to the slides. The slides are then incubated with the antibody in a moist chamber for 60 minutes, following which they are washed three times with PBS. To detect the bound primary antibody, two drops of a secondary biotinylated antibody (e.g., goat anti-mouse IgG antibody available from Zymed) are added to each slide for a 10-minute incubation in a moist chamber. The secondary antibody is washed off three times with PBS. To reveal the biotinylated secondary antibody, two drops of an horseradish peroxidase (HRP)-streptavidin conjugate (available from Zymed) are added for a 10-minute incubation in a moist chamber, followed by three washes in PBS. Finally, to detect the HRP-conjugated streptavidin, two drops of an aminoethylcarbazole (AEC Single Solution Chromogen/Substrate, Zymed) HRP substrate are added for a 6-minute incubation in a moist chamber, followed by three washed with PBS. Counterstaining is performed by dipping the slides for 25 seconds in a 2% of Hematoxylin solution (Sigma-Aldrich Corp., St Louis, Mo., USA, Cat. No. GHS-2-32) following which the slides were washed under tap water and covered with a coverslip.

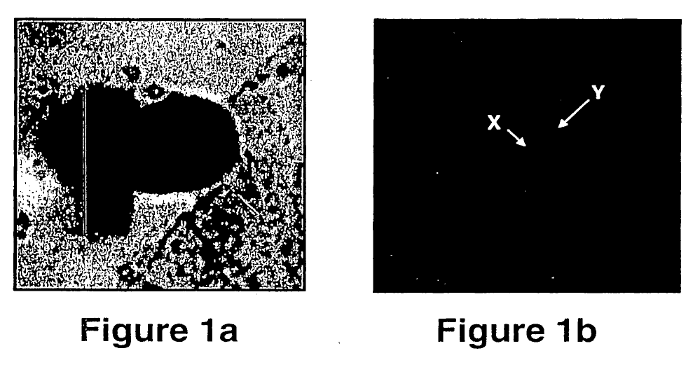

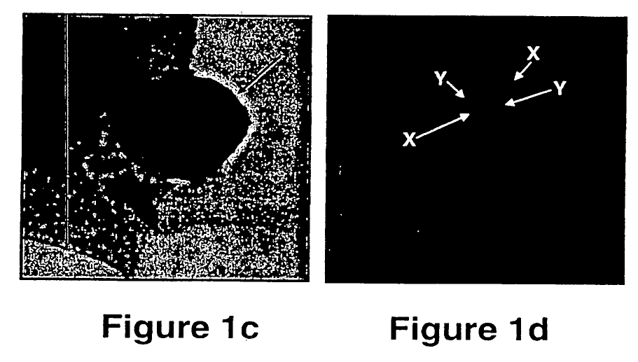

As is shown in FIG. 1-5 and Table 3 in Example 2 of the Examples section which follows, trophoblast cells were detected in 348 out of 396 transcervical specimens using the anti HLA-G antibody (MEM-G/l, Abcam, Cat. No. ab7759, Cambridge, UK), the anti PLAP antibody (Zymed, Cat. No. 18-0099, San Francisco, Calif., USA) and/or the CHL1 antibody (anti MCAM, CD146, Alexis Biochemicals).

It will be appreciated that following immunological staining, the immunologically-positive cells (i.e., trophoblasts) are viewed under a fluorescent or light microscope (depending on the staining method) and are preferably photographed using e.g., a CCD camera. In order to subject the same trophoblast cells of the same sample to further chromosomal and/or DNA analysis, the position (i.e., coordinate location) of such cells on the slide is stored in the microscope or a computer connected thereto for later reference. Examples of microscope systems which enable identification and storage of cell coordinates include the Bio View Duet™ (Bio View LtD, Rehovot, Israel), and the Applied Imaging System (Newcastle England), essentially as described in Merchant, F. A. and Castleman K. R. (Hum. Repr. Update, 2002, 8: 509-521).

As is mentioned before, once a trophoblast cell is identified within the trophoblast-containing cell sample it is subjected to in situ chromosomal and/or DNA analysis.

As used herein, “in situ chromosomal and/or DNA analysis” refers to the analysis of the chromosome(s) and/or the DNA within the cells, using fluorescent in situ hybridization (FISH), primed in situ labeling (PRINS), quantitative FISH (Q-FISH) and/or multicolor-banding (MCB).

According to the method of the present invention, the immunological staining and the in situ chromosomal and/or DNA analysis are effected sequentially on the same trophoblast-containing cell sample.

It will be appreciated that special treatments are required to make an already immunologically-stained cell amendable for a second staining method (e.g., FISH). Such treatments include for example, washing off the bound antibody (using e.g., water and a gradual ethanol series), exposing cell nuclei (using e.g., a methanol-acetic acid fixer), and digesting proteins (using e.g., Pepsin), essentially as described under the “Materials and Experimental Methods” section of Example 1 of the Examples section which follows and in Strehl S, Ambros P F (Cytogenet. Cell Genet. 1993, 63:24-8).

Methods of employing FISH analysis on interphase chromosomes are known in the art. Briefly, directly-labeled probes [e.g., the CEP X green and Y orange (Abbott cat no. 5J10-51)] are mixed with hybridization buffer (e.g., LSI/WCP, Abbott) and a carrier DNA (e.g., human Cot 1 DNA, available from Abbott). The probe solution is applied on microscopic slides containing e.g., transcervical cytospin specimens and the slides are covered using a coverslip. The probe-containing slides are denatured for 3 minutes at 70° C. and are further incubated for 48 hours at 37° C. using an hybridization apparatus (e.g., HYBrite, Abbott Cat. No. 2J11-04). Following hybridization, the slides are washed for 2 minutes at 72° C. in a solution of 0.3% NP-40 (Abbott) in 60 mM NaCl and 6 mM NaCitrate (0.4×SSC). Slides are then immersed for 1 minute in a solution of 0.1% NP-40 in 2×SSC at room temperature, following which the slides are allowed to dry in the dark. Counterstaining is performed using, for example, DAPI II counterstain (Abbott).

PRINS analysis has been employed in the detection of gene deletion (Tharapel S A and Kadandale J S, 2002. Am. J. Med. Genet. 107: 123-126), determination of fetal sex (Orsetti, B., et al., 1998. Prenat. Diagn. 18: 1014-1022), and identification of chromosomal aneuploidy (Mennicke, K. et al., 2003. Fetal Diagn. Ther. 18: 114-121).

Methods of performing PRINS analysis are known in the art and include for example, those described in Coullin, P. et al. (Am. J. Med. Genet. 2002, 107: 127-135); Findlay, I., et al. (J. Assist. Reprod. Genet. 1998, 15: 258-265); Musio, A., et al. (Genome 1998, 41: 739-741); Mennicke, K., et al. (Fetal Diagn. Ther. 2003, 18: 114-121); Orsetti, B., et al. (Prenat. Diagn. 1998, 18: 1014-1022). Briefly, slides containing interphase chromosomes are denatured for 2 minutes at 71° C. in a solution of 70% formamide in 2×SSC (pH 7.2), dehydrated in an ethanol series (70, 80, 90 and 100%) and are placed on a flat plate block of a programmable temperature cycler (such as the PTC-200 thermal cycler adapted for glass slides which is available from MJ Research, Waltham, Mass., USA). The PRINS reaction is usually performed in the presence of unlabeled primers and a mixture of dNTPs with a labeled dUTP (e.g., fluorescein-12-dUTP or digoxigenin-11-dUTP for a direct or indirect detection, respectively). Alternatively, or additionally, the sequence-specific primers can be labeled at the 5′ end using e.g., 1-3 fluorescein or cyanine 3 (Cy3) molecules. Thus, a typical PRINS reaction mixture includes sequence-specific primers (50-200 pmol in a 50 μl reaction volume), unlabeled dNTPs (0.1 mM of dATP, dCTP, dGTP and 0.002 mM of dTTP), labeled dUTP (0.025 mM) and Taq DNA polymerase (2 units) with the appropriate reaction buffer. Once the slide reaches the desired annealing temperature the reaction mixture is applied on the slide and the slide is covered using a cover slip. Annealing of the sequence-specific primers is allowed to occur for 15 minutes, following which the primed chains are elongated at 72° C. for another 15 minutes. Following elongation, the slides are washed three times at room temperature in a solution of 4×SSC/0.5% Tween-20 (4 minutes each), followed by a 4-minute wash at PBS. Slides are then subjected to nuclei counterstain using DAPI or propidium iodide. The fluorescently stained slides can be viewed using a fluorescent microscope and the appropriate combination of filters (e.g., DAPI, FITC, TRITC, FITC-rhodamin).

It will be appreciated that several primers which are specific for several targets can be used on the same PRINS run using different 5′ conjugates. Thus, the PRINS analysis can be used as a multicolor assay for the determination of the presence, and/or location of several genes or chromosomal loci.

In addition, as described in Coullin et al., (2002, Supra) the PRINS analysis can be performed on the same slide as the FISH analysis, preferably, prior to FISH analysis.

High-resolution multicolor banding (MCB) on interphase chromosomes—This method, which is described in detail by Lemke et al. (Am. J. Hum. Genet. 71: 1051-1059, 2002), uses YAC/BAC and region-specific microdissection DNA libraries as DNA probes for interphase chromosomes. Briefly, for each region-specific DNA library 8-10 chromosome fragments are excised using microdissection and the DNA is amplified using a degenerated oligonucleotide PCR reaction. For example, for MCB staining of chromosome 5, seven overlapping microdissection DNA libraries were constructed, two within the p arm and five within the q arm (Chudoba I., et al., 1999; Cytogenet. Cell Genet. 84: 156-160). Each of the DNA libraries is labeled with a unique combination of fluorochromes and hybridization and post-hybridization washes are carried out using standard protocols (see for example, Senger et al., 1993; Cytogenet. Cell Genet. 64: 49-53). Analysis of the multicolor-banding can be performed using the isis/mFISH imaging system (MetaSystems GmbH, Altlussheim, Germany). It will be appreciated that although MCB staining on interphase chromosomes was documented for a single chromosome at a time, it is conceivable that additional probes and unique combinations of fluorochromes can be used for MCB staining of two or more chromosomes at a single MCB analysis. Thus, this technique can be used along with the present invention to identify fetal chromosomal aberrations, particularly, for the detection of specific chromosomal abnormalities which are known to be present in other family members.

Quantitative FISH (Q-FISH)—In this method chromosomal abnormalities are detected by measuring variations in fluorescence intensity of specific probes. Q-FISH can be performed using Peptide Nucleic Acid (PNA) oligonucleotide probes. PNA probes are synthetic DNA mimics in which the sugar phosphate backbone is replaced by repeating N-(2-aminoethyl)glycine units linked by an amine bond and to which the nucleobases are fixed (Pellestor F and Paulasova P, 2004; Chromosoma 112: 375-380). Thus, the hydrophobic and neutral backbone enables high affinity and specific hybridization of the PNA probes to their nucleic acid counterparts (e.g., chromosomal DNA). Such probes have been applied on interphase nuclei to monitor telomere stability (Slijepcevic, P. 1998; Mutat. Res. 404:215-220; Henderson S., et al., 1996; J. Cell Biol. 134: 1-12), the presence of Fanconi aneamia (Hanson H, et al., 2001, Cytogenet. Cell Genet. 93: 203-6) and numerical chromosome abnormalities such as trisomy 18 (Chen C, et al., 2000, Mamm. Genome 10: 13-18), as well as monosomy, duplication, and deletion (Taneja K L, et al., 2001, Genes Chromosomes Cancer. 30: 57-63).

Alternatively, Q-FISH can be performed by co-hybridizing whole chromosome painting probes (e.g., for chromosomes 21 and 22) on interphase nuclei as described in Truong K et al, 2003, Prenat. Diagn. 23: 146-51.

Altogether, as is further shown in Table 3 and in Example 2 of the Examples section which follows, a successful FISH result was obtained in 92.45% of the trophoblast-containing transcervical specimens as confirmed by the karyotype results obtained using fetal cells of placental biopsies, amniocentesis or CVS.

Since the chromosomal and/or DNA analysis is performed on the same cell which was immunologically stained using a trophoblast-specific antibody, the method of the present invention can be used to determine fetal gender and/or identify at least one chromosomal abnormality in a fetus.

According to preferred embodiments of the present invention, the chromosomal abnormality can be chromosomal aneuploidy (i.e., complete and/or partial trisomy and/or monosomy), translocation, subtelomeric rearrangement, deletion, microdeletion, inversion and/or duplication (i.e., complete an/or partial chromosome duplication).

According to preferred embodiments of the present invention the trisomy detected by the present invention can be trisomy 21 [using e.g., the LSI 21q22 orange labeled probe (Abbott cat no. 5J13-02)], trisomy 18 [using e.g., the CEP 18 green labeled probe (Abbott Cat No. 5J10-18); the CEP® 18 (D18Z1, α satellite) Spectrum Orange™ probe (Abbott Cat No. 5J08-18)], trisomy 16 [using e.g., the CEP16 probe (Abbott Cat. No. 6J37-17)], trisomy 13 [using e.g., the LSI® 13 SpectrumGreen™ probe (Abbott Cat. No. 5J14-18)], and the XXY, XYY, or XXX trisomies which can be detected using e.g., the CEP X green and Y orange probe (Abbott cat no. 5J10-51); and/or the CEP®X SpectrumGreen™/CEP® Y (μ satellite) SpectrumOrange™ probe (Abbott Cat. No. 5J10-51).

It will be appreciated that using the chromosome-specific FISH probes, PRINS primers, Q-FISH and MCB staining various other trisomies and partial trisomies can be detected in fetal cells according to the teachings of the present invention. These include, but not limited to, partial trisomy 1q32-44 (Kimya Y et al., Prenat Diagn. 2002, 22:957-61), trisomy 9p with trisomy 10p (Hengstschlager M et al., Fetal Diagn Ther. 2002, 17:243-6), trisomy 4 mosaicism (Zaslav A L et al., Am J Med Genet. 2000, 95:381-4), trisomy 17p (De Pater J M et al., Genet Couns. 2000, 11:241-7), partial trisomy 4q26-qter (Petek E et al., Prenat Diagn. 2000, 20:349-52), trisomy 9 (Van den Berg C et al., Prenat. Diagn. 1997, 17:933-40), partial 2p trisomy (Siffroi J P et al., Prenat Diagn. 1994, 14:1097-9), partial trisomy 1q (DuPont B R et al., Am J Med Genet. 1994, 50:21-7), and/or partial trisomy 6p/monosomy 6q (Wauters J G et al., Clin Genet. 1993, 44:262-9).

The method of the present invention can be also used to detect several chromosomal monosomies such as, monosomy 22, 16, 21 and 15, which are known to be involved in pregnancy miscarriage (Munne, S. et al., 2004. Reprod Biomed Online. 8: 81-90)].

According to preferred embodiments of the present invention the monosomy detected by the method of the present invention can be monosomy X, monosomy 21, monosomy 22 [using e.g., the LSI 22 (BCR) probe (Abbott, Cat. No. 5J17-24)], monosomy 16 (using e.g., the CEP 16 (D16Z3) Abbott, Cat. No. 6J36-17) and monosomy 15 [using e.g., the CEP 15 (D15Z4) probe (Abbott, Cat. No. 6J36-15)].

It will be appreciated that several translocations and microdeletions can be asymptomatic in the carrier parent, yet can cause a major genetic disease in the offspring. For example, a healthy mother who carries the 15q11-q13 microdeletion can give birth to a child with Angelman syndrome, a severe neurodegenerative disorder. Thus, the present invention can be used to identify such a deletion in the fetus using e.g., FISH probes which are specific for such a deletion (Erdel M et al., Hum Genet. 1996, 97: 784-93).

Thus, the present invention can also be used to detect any chromosomal abnormality if one of the parents is a known carrier of such abnormality. These include, but not limited to, mosaic for a small supernumerary marker chromosome (SMC) (Giardino D et al., Am J Med Genet. 2002, 111:319-23); t(11; 14)(p15; p13) translocation (Benzacken B et al., Prenat Diagn. 2001, 21:96-8); unbalanced translocation t(8; 11) (p23.2; p15.5) (Fert-Ferrer S et al., Prenat Diagn. 2000, 20:511-5); 11q23 microdeletion (Matsubara K, Yura K. Rinsho Ketsueki. 2004, 45:61-5); Smith-Magenis syndrome 17p11.2 deletion (Potocki L et al., Genet Med. 2003, 5:430-4); 22q13.3 deletion (Chen C P et al., Prenat Diagn. 2003, 23:504-8); Xp22.3. microdeletion (Enright F et al., Pediatr Dermatol. 2003, 20:153-7); 10p14 deletion (Bartsch O, et al., Am J Med Genet. 2003, 117A:1-5); 20p microdeletion (Laufer-Cahana A, Am J Med Genet. 2002, 112:190-3.), DiGeorge syndrome [del(22) (q11.2q11.23)], Williams syndrome [7q11.23 and 7q36 deletions, Wouters C H, et al., Am J Med Genet. 2001, 102:261-5.]; 1p36 deletion (Zenker M, et al., Clin Dysmorphol. 2002, 11:43-8); 2p microdeletion (Dee S L et al., J Med Genet. 2001, 38:E32); neurofibromatosis type 1 (17q11.2 microdeletin, Jenne D E, et al., Am J Hum Genet. 2001, 69:516-27); Yq deletion (Toth A, et al., Prenat Diagn. 2001, 21:253-5); Wolf-Hirschhorn syndrome (WHS, 4p16.3 microdeletion, Rauch A et al., Am J Med Genet. 2001, 99:338-42); 1p36.2 microdeletion (Finelli P, Am J Med Genet. 2001, 99:308-13); 11q14 deletion (Coupry I et al., J Med Genet. 2001, 38:35-8); 19q13.2 microdeletion (Tentler D et al., J Med Genet. 2000, 37:128-31); Rubinstein-Taybi (16p13.3 microdeletion, Blough R I, et al., Am J Med Genet. 2000, 90:29-34); 7p21 microdeletion (Johnson D et al., Am J Hum Genet. 1998, 63:1282-93); Miller-Dieker syndrome (17p13.3), 17p11.2 deletion (Juyal R C et al., Am J Hum Genet. 1996, 58:998-1007); 2q37 microdeletion (Wilson L C et al., Am J Hum Genet. 1995, 56:400-7).

The present invention can be used to detect inversions [e.g., inverted chromosome X (Lepretre, F. et al., Cytogenet. Genome Res. 2003. 101: 124-129; Xu, W. et al., Am. J. Med. Genet. 2003. 120A: 434-436), inverted chromosome 10 (Helszer, Z., et al., 2003. J. Appl. Genet. 44: 225-229)], cryptic subtelomeric chromosome rearrangements (Engels, H., et al., 2003. Eur. J. Hum. Genet. 11: 643-651; Bocian, E., et al., 2004. Med. Sci. Monit. 10: CR143-CR151), and/or duplications (Soler, A., et al., Prenat. Diagn. 2003. 23: 319-322).

Thus, the teachings of the present invention can be used to identify chromosomal aberrations in a fetus without subjecting the mother to invasive and risk-carrying procedures.

For example, in order to determine fetal gender and/or the presence of a Down syndrome fetus (i.e., trisomy 21) according to the teachings of the present invention, transcervical cells are obtained from a pregnant woman at 7th to the 11th weeks of gestation using a Pap smear cytobrush. The cells are suspended in RPMI-1640 medium tissue culture medium (Beth Haemek, Israel) in the presence of 1% Penicillin Streptomycin antibiotic, and cytospin slides are prepared using a Cytofunnel Chamber Cytocentrifuge (Thermo-Shandon, England) according to manufacturer's instructions. Cytospin slides are dehydrated in 95% alcohol until immunohistochemical analysis is performed.

Prior to immunohistochemistry, cytospin slides are hydrated in 70% alcohol and water, washed with PBS, treated with 3% hydrogen peroxide followed by three washes in PBS and incubated with a blocking reagent (from the Zymed HISTOSTAIN®-PLUS Kit, Cat No. 858943). An HLA-G antibody (mAb 7759, Abcam Ltd., Cambridge, UK) is applied on the slides according to manufacturer's instructions for a 60-minutes incubation followed by 3 washes in PBS. A secondary biotinylated goat anti-mouse IgG antibody (Zymed HISTOSTAIN®-PLUS Kit, Cat No. 858943) is added to the slide for a 10-minute incubation followed by three washes in PBS. The secondary antibody is then retrieved using the HRP-streptavidin conjugate (Zymed HISTOSTAIN®-PLUS Kit, Cat No. 858943) and the aminoethylcarbazole (AEC Single Solution Chromogen/Substrate, Zymed) HRP substrate according to manufacturer's instructions. Counterstaining is performed using Hematoxylin solution (Sigma-Aldrich Corp., St Louis, Mo., USA, Cat. No. GHS-2-32). The immunologically stained transcervical samples are viewed and photographed using a light microscope (AX-70 Provis, Olympus, Japan) and a CCD camera (Applied Imaging, Newcastle, England) connected to it, and the position of HLA-G positive trophoblast cells are marked using the microscope coordination.

To remove antibody's residual staining, stained slides are immersed in 2% ammonium hydroxide (diluted in 70% alcohol), washed for one minute in distilled water, immersed for a few seconds in 100% acetic acid and washed for one minute in distilled water. Prior to FISH analysis slides containing HLA-G-positive cells are dehydrated in 70% and 100% ethanol, and fixed for 10 minutes in a methanol-acetic acid (in a 3:1 ratio) fixer solution. Slides are then washed in a warm solution (at 37° C.) of 2×SSC, fixed in 0.9% of formaldehyde in PBS and washed in PBS. Prior to FISH analysis, slides are digested with a Pepsin solution (0.15% in 0.01 N HCl), dehydrated in an ethanol series and dried.

For the determination of fetal gender, 7 μl of the LSI/WCP hybridization buffer (Abbott) are mixed with 1 μl of the directly-labeled CEP X green and Y orange probes containing the centromere regions Xp11.1-q11.1 (DXZ1) and Yp11.1-q11.1 (DYZ3) (Abbott Cat no. 5J10-51), 1-2 μl of human Cot 1 DNA (1 μg/μl Abbott, Cat No. 06J31-001) and 2 μl of purified double-distilled water. The probe-hybridization solution is centrifuged for 1-3 seconds and 11 μl of the probe-hybridization solution is applied on each slide, following which, the slides are immediately covered using a coverslip. Slides are then denatured for 3 minutes at 70° C. and further incubated at 37° C. for 48 hours in the HYBrite apparatus (Abbott Cat. No. 2J11-04). Following hybridization, slides are washed in 0.3% NP-40 in 0.4×SSC, followed by 0.1% NP-40 in 2×SSC and are allowed to dry in the dark. Counterstaining is performed using DAPI II (Abbott). Slides are then viewed using a fluorescent microscope (AX-70 Provis, Olympus, Japan) according to the previously marked positions of the HLA-G-positive cells and photographed.

For the determination of the presence or absence of a Down syndrome fetus, following the first set of FISH analysis the slides are washed in 1×SSC (20 minutes, room temperature) following which they are dipped for 10 seconds in purified double-distilled water at 71° C. Slides are then dehydrated in an ethanol series and dried. Hybridization is effected using the LSI 21q22 orange labeled probe containing the D21S259, D21S341 and D21S342 loci within the 21q22.13 to 21q22.2 region (Abbott cat no. 5J13-02) and the same hybridization and washing conditions as used for the first set of FISH probes. The FISH signals obtained following the second set of FISH probes are viewed using the fluorescent microscope and the same coordination of HLA-G positive trophoblast cells.

The use of FISH probes for chromosomes 13, 18, 21, X and Y on interphase chromosomes was found to reduce the residual risk for a clinically significant abnormality from 0.9-10.1% prior to the interphase FISH assay, to 0.6-1.5% following a normal interphase FISH pattern [Homer J, et al., 2003. Residual risk for cytogenetic abnormalities after prenatal diagnosis by interphase fluorescence in situ hybridization (FISH). Prenat Diagn. 23: 566-71]. Thus, the teachings of the present invention can be used to significantly reduce the risk of having clinically abnormal babies by providing an efficient method of prenatal diagnosis.

It will be appreciated that the trophoblast cell of the present invention can be also subjected to DNA analysis in order to identify single gene disorders (e.g., cystic fibrosis, Tay-Sachs disease, Canavan disease, Gaucher disease, Familial Dysautonomia, Niemann-Pick disease, Fanconi anemia, Ataxia telaugiestasia, Bloom syndrome, Familial Mediterranean fever (FMF), X-linked spondyloepiphyseal dysplasia tarda, factor XI), DNA-methylation related disorders [e.g., imprinting disorders such as Angelman Syndrome, Prader-Willi Syndrome, Beckwith-Wiedemann syndrome, Myoclonus-dystonia syndrome (MDS)], as well as disorders which are caused by minor chromosomal aberrations (e.g., minor trisomy mosaicisms, duplication sub-telomeric regions, interstitial deletions or duplications) which are below the detection level of conventional in situ chromosomal and/or DNA analysis methods (i.e., FISH, Q-FISH, MCB and PRINS).

Thus, according to another aspect of the present invention there is provided a method of determining fetal gender and/or identifying at least one chromosomal and/or DNA abnormality of a fetus.

The phrase “DNA abnormality” refers to a single nucleotide substitution, deletion, insertion, micro-deletion, micro-insertion, short deletion, short insertion, multinucleotide substitution, and abnormal DNA methylation and loss of imprint (LOI). Such a DNA abnormality can be related to an inherited genetic disease such as a single-gene disorder (e.g., cystic fibrosis, Canavan, Tay-Sachs disease, Gaucher disease, Familial Dysautonomia, Niemann-Pick disease, Fanconi anemia, Ataxia telaugiestasia, Bloom syndrome, Familial Mediterranean fever (FMF), X-linked spondyloepiphyseal dysplasia tarda, factor XI), an imprinting disorder [e.g., Angelman Syndrome, Prader-Willi Syndrome, Beckwith-Wiedemann syndrome, Myoclonus-dystonia syndrome (MDS)], or to predisposition to various cancer diseases (e.g., mutations in the BRCA1 and BRCA2 genes).

The method is effected by subjecting at least one stained trophoblast cell to a genetic analysis.

The phrase “genetic analysis” as used herein refers to any chromosomal, DNA and/or RNA-based analysis which can detect chromosomal, DNA and/or gene expression abnormalities, respectively in a cell of an individual (i.e., in the trophoblast cell of the present invention).

As is mentioned hereinabove, major and minor chromosomal abnormalities can be detected in interphase chromosomes using conventional methods such as FISH, Q-FISH, MCB and PRINS. However, the identification of some subtle chromosomal abnormalities require the application of DNA-based detection methods such as comparative genome hybridization (CGH).

Comparative Genome Hybridization (CGH)—is based on a quantitative two-color fluorescence in situ hybridization (FISH) on metaphase chromosomes. In this method a test DNA (e.g., DNA extracted from the trophoblast cell of the present invention) is labeled in one color (e.g., green) and mixed in a 1:1 ratio with a reference DNA (e.g., DNA extracted from a control cell) which is labeled in a different color (e.g., red). Methods of amplifying and labeling whole-genome DNA are well known in the art (see for example, Wells D, et al., 1999; Nucleic Acids Res. 27: 1214-8). Briefly, genomic DNA is amplified using a degenerate oligonucleotide primer [e.g., 5′-CCGACTCGAGNNNNNNATGTGG, SEQ ID NO:11 (Telenius, H., et al., 1992; Genomics 13:718-25)] and the amplified DNA is labeled using e.g., the Spectrum Green-dUTP (for the test DNA) or the Spectrum Red-dUTP (for the reference DNA). The mixture of labeled DNA samples is precipitated with Cot1 DNA (Gibco-BRL) and resuspended in an hybridization mixture containing e.g., 50% formamide, 2×SSC, pH 7 and 10% dextrane sulfate. Prior to hybridization, the labeled DNA samples (i.e., the probes) are denatured for 10 minutes at 75° C. and allowed to cool at room temperature for 2 minutes. Likewise, the metaphase chromosome spreads are denatured using standard protocols (e.g., dehydration in a series of ethanol, denaturation for 5 minutes at 75° C. in 70% formamide and 2×SSC). Hybridization conditions include incubation at 37° C. for 25-30 hours in a humidified chamber, following by washes in 2×SSC and dehydration using an ethanol series, essentially as described elsewhere (Wells, D., et al., 2002; Fertility and Sterility, 78: 543-549). Hybridization signal is detected using a fluorescence microscope and the ratio of the green-to-red fluorescence can be determined using e.g., the Applied Imaging (Santa Clara, Calif.) computer software. If both genomes are equally represented in the metaphase chromosomes (i.e., no deletions, duplication or insertions in the DNA derived from the trophoblast cell) the labeling on the metaphase chromosomes is orange. However, regions which are either deleted or duplicated in the trophoblast cell are stained with red or green, respectively.

It will be appreciated that since the cell of the present invention (i.e., the trophoblast cell) is processed according to the method of the present invention to include interphase chromosomes, the metaphase chromosomes used by the CGH method are derived from the reference cell (i.e., a normal individual) having a karyotype of either 46, XY or 46, XX.

DNA array-based comparative genomic hybridization (CGH-array)—This method, which is fully described in Hu, D. G., et al., 2004, Mol. Hum. Reprod. 10: 283-289, is a modified version of CGH and is based on the hybridization of a 1:1 mixture of the test and reference DNA probes on an array containing chromosome-specific DNA libraries. Methods of preparing chromosome-specific DNA libraries are known in the art (see for example, Bolzer A., et al., 1999; Cytogenet. Cell. Genet. 84: 233-240). Briefly, single chromosomes are obtained using either microdissection or flow-sorting and the genomic DNA of each of the isolated chromosomes is PCR-amplified using a degenerated oligonucleotide primer. To remove repetitive DNA sequences, the amplified DNA is subjected to affinity chromatography in combination with negative subtraction hybridization (using e.g., human Cot-1 DNA or centromere-specific repetitive sequence as subtractors), essentially as described in Craig J M., et al., 1997; Hum. Genet. 100: 472-476. Amplified chromosome-specific DNA libraries are then attached to a solid support [(e.g., SuperAmine slides (TeleChem, USA)], dried, baked and washed according to manufacturer's recommendation. Labeled genomic DNA probes (a 1:1 mixture of the test and reference DNAs) are mixed with non-specific carrier DNA (e.g., human Cot-1 and/or salmon sperm DNA, Gibco-BRL), ethanol-precipitated and re-suspended in an hybridization buffer such as 50% deionized formamide, 2×SSC, 0.1% SDS, 10% Dextran sulphate and 5×Denhardt's solution. The DNA probes are then denatured (80° C. for 10 minutes), pre-annealed (37° C. for 80 minutes) and applied on the array for hybridization of 15-20 hours in a humid incubator. Following hybridization the arrays are washed twice for 10 minutes in 50% formamide/2×SSC at 45° C. and once for 10 minutes in 1×SSC at room temperature, following which the arrays are rinsed three times in 18.2 MΩ deionized water. The arrays are then scanned using any suitable fluorescence scanner such as the GenePix 4000B microarray reader (Axon Instruments, USA) and analyzed using the GenePix Pro. 4.0.1.12 software (Axon).

The DNA-based CGH-array technology was shown to confirm fetal abnormalities detected using conventional G-banding and to identify additional fetal abnormalities such as mosaicism of trisomy 20, duplication of 10q telomere region, interstitial deletion of chromosome 9p and interstitial duplication of the PWS region on chromosome 15q which is implicated in autism if maternally inherited (Schaeffer, A. J., et al., 2004; Am. J. Hum. Genet. 74: 1168-1174), unbalanced translocation (Klein O D, et al., 2004, Clin Genet. 65: 477-82), unbalanced subtelomeric rearrangements (Ness G O et al., 2002, Am. J. Med. Genet. 113: 125-36), unbalanced inversions and/or chromosomal rearrangements (Daniely M, et al., 1999; Cytogenet Cell Genet. 86: 51-5).

The identification of single gene disorders, imprinting disorders, and/or predisposition to cancer can be effected using any method suitable for identification of at least one nucleic acid substitution such as a single nucleotide polymorphism (SNP).

Direct sequencing of a PCR product: This method is based on the amplification of a genomic sequence using specific PCR primers in a PCR reaction following by a sequencing reaction utilizing the sequence of one of the PCR primers as a sequencing primer. Sequencing reaction can be performed using, for example, the Applied Biosystems (Foster City, Calif.) ABI PRISM® BigDye™ Primer or BigDye™ Terminator Cycle Sequencing Kits.

Restriction fragment length polymorphism (RFLP): This method uses a change in a single nucleotide which modifies a recognition site for a restriction enzyme resulting in the creation or destruction of an RFLP. RFLP can be used on a genomic DNA using a labeled probe (i.e., Southern Blot RFLP) or on a PCR product (i.e., PCR-RFLP).

For example, RFLP can be used to detect the cystic fibrosis-causing mutation, ΔF508 [deletion of a CTT at nucleotide 1653-5, GenBank Accession No. M28668, SEQ ID NO:1; Kerem B, et al., Science. 1989, 245: 1073-80] in a genomic DNA derived from the isolated trophoblast cell of the present invention. Briefly, genomic DNA is amplified using the forward [5′-GCACCATTAAAGAAAATATGAT (SEQ ID NO:2)] and the reverse [5′-CTCTTCTAGTTGGCATGCT (SEQ ID NO:3)] PCR primers, and the resultant 86 or 83 bp PCR products of the wild-type or AF508 allele, respectively are subjected to digestion using the DpnI restriction enzyme which is capable of differentially digesting the wild-type PCR product (resulting in a 67 and 19 bp fragments) but not the CTT-deleted allele (resulting in a 83 bp fragment).

Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

Allele specific oligonucleotide (ASO): In this method, an allele-specific oligonucleotide (ASO) is designed to hybridize in proximity to the substituted nucleotide, such that a primer extension or ligation event can be used as the indicator of a match or a mis-match. Hybridization with radioactively labeled allelic specific oligonucleotides (ASO) also has been applied to the detection of specific SNPs (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles.

It will be appreciated that ASO can be applied on a PCR product generated from genomic DNA. For example, to detect the A455E mutation (C1496→A in SEQ ID NO:1) which causes cystic fibrosis, trophoblast genomic DNA is amplified using the 5′-TAATGGATCATGGGCCATGT (SEQ ID NO:4) and the 5′-ACAGTGTTGAATGTGGTGCA (SEQ ID NO:5) PCR primers, and the resultant PCR product is subjected to an ASO hybridization using the following oligonucleotide probe: 5′-GTTGTTGGAGGTTGCT (SEQ ID NO:6) which is capable of hybridizing to the thymidine nucleotide at position 1496 of SEQ ID NO:1. As a control for the hybridization, the 5′-GTTGTTGGCGGTTGCT (SEQ ID NO:7) oligonucleotide probe is applied to detect the presence of the wild-type allele essentially as described in Kerem B, et al., 1990, Proc. Natl. Acad. Sci. USA, 87:8447-8451).

Allele-specific PCR—In this method the presence of a single nucleic acid substitution is detected using differential extension of a mutant and/or wild-type—specific primer on one hand, and a common primer on the other hand. For example, the detection of the cystic fibrosis Q493X mutation (C1609→T in SEQ ID NO:1) is performed by amplifying genomic DNA (derived from the trophoblast cell of the present invention) using the following three primers: the common primer (i.e., will amplify in any case): 5′-GCAGAGTACCTGAAACAGGA (SEQ ID NO:8); the wild-type primer (i.e., will amplify only the cytosine-containing wild-type allele): 5′-GGCATAATCCAGGAAAACTG (SEQ ID NO:9); and the mutant primer (i.e., will amplify only the thymidine-containing mutant allele): 5′-GGCATAATCCAGGAAAACTA (SEQ ID NO: 10), essentially as described in Kerem, 1990 (Supra).

Methylation-specific PCR (MSPCR)— This method is used to detect specific changes in DNA methylation which are associated with imprinting disorders such Angelman or Prader-Willi syndromes. Briefly, the DNA is treated with sodium bisulfite which converts the unmethylated, but not the methylated, cytosine residues to uracil. Following sodium bisulfite treatment the DNA is subjected to a PCR reaction using primers which can anneal to either the uracil nucleotide-containing allele or the cytosine nucleotide-containing allele as described in Buller A., et al., 2000, Mol. Diagn. 5: 239-43.

Pyrosequencing™ analysis (Pyrosequencing, Inc. Westborough, Mass., USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated.

Acycloprime™ analysis (Perkin Elmer, Boston, Mass., USA): This technique is based on fluorescent polarization (FP) detection. Following PCR amplification of the sequence containing the substituted nucleic acid (causing the DNA abnormality in the fetus), excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the substituted nucleic acid. The terminator(s) added are identified by their increased FP and represent the allele(s) present in the original DNA sample. The Acycloprime process uses Acyclopol™, a novel mutant thermostable polymerase from the Archeon family, and a pair of AcycloTerminators™ labeled with R110 and TAMRA, representing the possible alleles for the substituted nucleic acid. AcycloTerminator™ non-nucleotide analogs are biologically active with a variety of DNA polymerases. Similarly to 2′,3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutants have for derivatized 2′,3′-dideoxynucleotide terminators.

Reverse dot blot: This technique uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized colorimetrically after hybridization through the use of streptavidin horseradish peroxidase incubation followed by development using tetramethylbenzidine and hydrogen peroxide, or via chemiluminescence after incubation with avidin alkaline phosphatase conjugate and a luminous substrate susceptible to enzyme activation, such as CSPD, followed by exposure to x-ray film.

It will be appreciated that advances in the field of SNP detection have provided additional accurate, easy, and inexpensive large-scale genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci USA. 88: 7276-80), as well as various DNA “chip” technologies such as the GeneChip microarrays (e.g., Affymetrix SNP chips) which are disclosed in U.S. Pat. Appl. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S, and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) and MassArray (Leushner J, Chiu N H, 2000. Mol Diagn. 5: 341-80).

Nucleic acid substitutions can be also identified in mRNA molecules derived from the isolated trophoblast cell of the present invention. Such mRNA molecules are first subjected to an RT-PCR reaction following which they are either directly sequenced or be subjected to any of the SNP detection methods described hereinabove.

It will be appreciated that in order to subject at least one stained trophoblast cell to any of the genetic analysis methods described hereinabove, the method further comprising a step of isolating the stained trophoblast cell prior to being subjected to the genetic analysis.

As used herein, the term “isolating” refers to a physical isolation of a trophoblast cell from a heterogeneous population of cells. Trophoblasts cells can be isolated from a maternal cell sample (e.g., blood, transcervical specimens) using a variety of antigen-based methods as described above. Alternatively, trophoblast cells can be isolated in situ (i.e., from a microscopic slide containing such cells) using, for example, laser-capture microdissection.

Laser-capture microdissection of cells is used to selectively isolate a specific cell type from a heterogeneous cell population contained on a slide. Methods of using laser-capture microdissection are known in the art (see for example, U.S. Pat. Appl. No. 20030227611 to Fein, Howard et al., Micke P, et al., 2004. J. Pathol., 202: 130-8; Evans E A, et al., 2003. Reprod. Biol. Endocrinol. 1: 54; Bauer M, et al. 2002. Paternity testing after pregnancy termination using laser microdissection of chorionic villi. Int. J. Legal Med. 116: 39-42; Fend, F. and Raffeld, M. 2000, J. Clin. Pathol. 53: 666-72).

For example, a trophoblast-containing cell sample (e.g., a cytospin slide of transcervical cells) is contacted with a selectively activated surface [e.g., a thermoplastic membrane such as a polyethylene membrane (PEN slides; C. Zeiss, Thornwood, N.Y.)] capable of adhering to a specific cell upon laser activation. The cell sample is subjected to a differential staining such as an immunological staining (using for example, an HLA-G, PLAP and/or CHL1 antibodies) essentially as described in Example 1 and 2 of the Example section which follows. Following staining, the cell sample is viewed using a microscope to identify the differentially stained trophoblast cells (i.e., HLA-G, PLAP and/or CHL1-positive cells, respectively). Once identified, a laser beam routed through an optic fiber [e.g., using the PALM Microbeam system (PALM Microlaser Technologies AG, Bernreid, Germany)] activates the surface which adheres to the selected trophoblast cell. The laser beam uses the ultraviolet (UV, e.g., 337 nm), the far-UV (200-315 nm) and the near-UV (315-400 nm) ray regions which are suitable for the further isolation of DNA, RNA or proteins from the microdissected cell. Following dissection (i.e., the cutting off of the cell), the laser beam blows off the cut cell into a recovery cap of a microtube, essentially as illustrated in Tachikawa T and Irie T, 2004, Med. Electron Microsc., 37: 82-88. For a genetic analysis, the DNA of the isolated trophoblast cell can be extracted using e.g., the alkaline lysis or the proteinase K protocols which are described in Rook M, et al., 2004, Am. J. of Pathology, 164: 23-33.

It will be appreciated that prior to isolating, the trophoblast cell needs to be identified. Current methods of identifying trophoblast cells include the immunological staining methods described hereinabove and in the Examples section which follows and an RNA in situ hybridization (RNA-ISH) staining method which uses a probe specific to a trophoblast-specific RNA transcript.

The trophoblast-specific RNA transcript of the present invention can be any RNA transcript which is expressed by the trophoblast cell. Examples include, but are not limited to, H19 (Lin W L, et al., 1999, Mech. Dev. 82: 195-7), HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

According to preferred embodiments of the present invention the probe used by the present invention can be any RNA molecule (e.g., RNA oligonucleotide, an in vitro transcribed RNA molecule), DNA molecule (e.g., oligonucleotide, cDNA molecule, genomic molecule) and/or an analogue thereof [e.g., peptide nucleic acid (PNA)] which is specific to the trophoblast-specific RNA transcript of the present invention. Methods of preparing such probes are well known in the arts.

RNA in situ hybridization stain: In this method a DNA, RNA or oligonucleotide probe is attached to a specific RNA molecule (e.g., a trophoblast-specific RNA transcript) present in the cells. The hybridization can take place in a cell suspension (as described in Lev-Lehman E, et al., 1997, Blood, 89: 3644-53) or on cells which are fixed to a microscopic slide. In any case, the cells are fixed using, e.g., formaldehyde or paraformaldehyde, to preserve the cellular structure and to prevent the RNA molecules from being degraded. Following fixation, an hybridization buffer containing the labeled probe (e.g., biotinylated or fluorescently labeled probe) is applied on the cells. The hybridization buffer includes reagents such as formamide and salts (e.g., sodium chloride and sodium citrate) which enable specific hybridization of the probe with its target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off (or removed via several cycles of centrifugation and resuspension) and the cells are subjected to a colorimetric reaction or a fluorescence microscope to reveal the signals generated by the bound probe.

For example, trophoblast cells have been identified using a probe specific to the HLA-G transcript (for further details see U.S. Pat. No. 5,750,339).

It will be appreciated that following RNA-ISH the cells can be further subjected to an in situ chromosomal and/or DNA analysis as described hereinabove. To enable efficient penetration of probe to the cell nuclei, the RNA-ISH stained cells are preferably fixed (using, for example, a methanol-acetic acid fixer solution) and treated with an enzyme such as Pepsin, which is capable of degrading all cellular structures. Noteworthy is that if the RNA-ISH staining is performed on cells in suspension the stained cells should be placed on microscopic slides (using e.g., cytospinning) prior to being subjected to the in situ chromosomal and/or DNA analysis. Those of skills in the art are capable of adjusting various treatment protocols (i.e., fixation and digestion) according to the type of cells and probes used.

The signal obtained using the RNA-ISH probe can be developed prior to the in situ chromosomal staining (e.g., FISH, Q-FISH) or simultaneously with the in situ chromosomal staining (e.g., using a biotinylated probe for the RNA-ISH staining and a directly labeled fluorescent probe for the FISH analysis).

Thus, following RNA-ISH the stained cells can be subjected to in situ chromosomal and/or DNA analysis, or can be isolated (using e.g., laser micro-dissection as described hereinabove) and be subjected to any method of the genetic analysis methods such as CGH and PCR-RFLP which are described hereinabove.

Altogether, the teachings of the present invention can be used to detect chromosomal and/or DNA abnormalities in a fetus by subjecting trophoblast cells obtained from transcervical cells to a trophoblast-specific immunological or RNA-ISH staining followed by an in situ chromosomal (e.g., FISH, MCB) and/or DNA (e.g., PRINS, Q-FISH) analysis or by isolating stained trophoblast cells and further subjecting them to any method of genetic analysis (e.g., CGH or any PCR-based detection method).

Briefly, in order to determine chromosomal aberrations and the presence of a cystic fibrosis (CF)—causing mutation in a fetus, trophoblast-containing cell samples (e.g., transcervical cells) are subjected to an RNA-ISH staining using an RNA oligonucleotide (e.g., 5′-biotinylated 2′-O-methyl-RNA) designed to hybridize with the H19 RNA transcript (e.g., the 5′-CGUAAUGGAAUGCUUGAAGGCUGC UCCGUGAUGUCGGUCGGAGCUUCCAG-3′ (SEQ ID NO:12) oligonucleotide. Following hybridization, the cells are viewed under a microscope and the trophoblast cells which are identified by the H19 RNA labeling are micro-dissected and isolated. To detect the presence of the CF-causing mutation, the DNA is extracted from the isolated trophoblast cells using methods known in the arts and is subjected to a PCR-RFLP analysis as described hereinabove. To detect chromosomal aberrations (such as trisomies, duplications, deletions) the DNA extracted from the trophoblast cell is labeled using e.g., the Spectrum Green-dUTP and is mixed in a 1:1 ratio with a reference DNA (obtained from a normal individual, i.e., 46, XX or 46, XY) which is labeled using the Spectrum Red-dUTP and the mixture of probes is applied on either metaphase chromosomes derived from a normal individual or on a CGH-array, as described hereinabove.

In order to determine chromosomal abnormalities in a fetus, the RNA-ISH-positive trophoblast cells (obtained using e.g., the PLAC1 or H19 probes) are dehydrated in 70% and 100% ethanol, and fixed for 10 minutes in a methanol-acetic acid (in a 3:1 ratio) fixer solution. Slides are then washed in a warm solution (at 37° C.) of 2×SSC, fixed in 0.9% of formaldehyde in PBS and washed in PBS. Prior to the hybridization with the FISH probes the slides are digested with a Pepsin solution (0.15% in 0.01 N HCl), dehydrated in an ethanol series and dried. Following FISH analysis, the trophoblast-stained cells can be subjected to laser micro-dissection and the DNA of the isolated trophoblast can be further subjected to CGH on either metaphase chromosome derived from a normal individual (i.e., 46, XX or 46, XY) or on a CGH-array. Alternatively, for the detection of a single gene disorder or an imprinting disorder, following FISH analysis the DNA of the isolated stained trophoblast is subjected to any of the PCR-based genetic analysis methods (e.g., ASO, PCR-RFLP, MS-PCR and the like).

Alternatively, prenatal diagnosis of a fetus can be effected by subjecting the transcervical cells to an immunological staining using the HLA-G, PLAP and/or CHL1 antibodies followed by an in situ chromosomal and/or DNA analysis (e.g., using PRINS and FISH, MCB or Q-FISH). The stained cells are isolated using laser microdissection and the DNA of the isolated trophoblast is subjected to either a CGH analysis (using CGH on metaphase chromosomes or a CGH-array) or to any of the SNP detection methods which are described hereinabove.

Optionally, following the immunological staining the stained trophoblast cell is isolated using laser microdissection and the DNA of the isolated trophoblast cell is subjected to a CGH-array or CGH analysis on metaphase chromosomes.

Prenatal paternity testing is currently performed on DNA samples derived from CVS and/or amniocentesis cell samples using PCR-based or RFLP analyses (Strom C M, et al., Am J Obstet Gynecol. 1996, 174: 1849-53; Yamada Y, et al., 2001. J Forensic Odontostomatol. 19: 1-4).

It will be appreciated that prenatal paternity testing can also be performed on trophoblast cells present in transcervical and/or intrauterine specimens using laser-capture microdissection.

Thus, according to another aspect of the present invention there is provided a method of determining a paternity of a fetus.

As used herein, the phrase “paternity” refers to the likelihood that a potential father of a specific fetus is the biological father of that fetus.

The method is effected by identifying and isolating the trophoblast cell of the present invention as described hereinabove (via and immunological staining and/or an RNA-ISH staining followed by laser capture microdissection), and subjecting the isolated trophoblast cell to a genetic analysis capable of detecting polymorphic markers of the fetus, and comparing the fetal polymorphic markers to a set of polymorphic markers obtained from a potential father.

As used herein, the phrase “polymorphic markers” refers to any nucleic acid change (e.g., substitution, deletion, insertion, inversion), variable number of tandem repeats (VNTR), short tandem repeats (STR), minisatellite variant repeats (MVR) and the like.

The polymorphic markers of the present invention can be determined using a variety of methods known in the arts, such as RFLP, PCR, PCR-RFLP and any of the SNP detection methods which are described hereinabove. For example, polymorphic markers used in paternity testing include the minisatellite variant repeats (MVR) at the MS32 (D1S8) or MS31A (D7S21) loci (Tamaki, K et al., 2000, Forensic Sci. Int. 113: 55-62)], the short tandem repeats (STR) at the DlS80 loci (Ceacareanu AC, Ceacareanu B, 1999, Roum. Arch. Microbiol. Immunol. 58: 281-8], the DXYS156 loci (Cali F, et al., 2002, Int. J. Legal Med. 116: 133-8), the “myo” and PYNH24 RFLP-probes [Strom C M, et al., (Supra) and Yamada Y, et al., (Supra)] and/or oligotyping of variable regions such as the HLA-II (Arroyo E, et al., 1994, J. Forensic Sci. 39: 566-72).

Thus, the teachings of the present invention can be used to determine the paternity of a fetus using transcervical cells from a pregnant mother. Briefly, a trophoblast cell is identified using an immunological staining (using e.g., an HLA-G, PLAP and/or CHL1 antibody) or an ISH-RNA staining (using e.g., a probe directed against the H19, PLAC1, PLAC8 and/or PLAC9 RNA transcripts), and is isolated using laser capture microdissection. The DNA of the isolated trophoblast is then extracted using, for example, proteinase K digestion and subjected to a genetic analysis of polymorphic markers such as the D1S80 (MCT118) marker, using the forward: 5′-GAAACTGGCCTCCAAACACTGCCCGCCG (SEQ ID NO:13) or the reverse: 5′-GTCTTGTTGGAGATGCACGTGCCCCTTGC (SEQ ID NO:14) PCR primers, and/or the MS32 and/or the MS31A loci [as described in Tamaki, 2000 (Supra)]. The polymorphic markers of the fetal DNA (i.e., the DNA isolated from the trophoblast cell of the present invention) are compared to the set of polymorphic markers obtained from the potential father (and preferably also from the mother) and the likelihood of the potential father to be the biological father is calculated using methods known in the art.

While further reducing the present invention to practice, the present inventors have uncovered that incubation of prestained slides with ammonium hydroxide significantly improves the results of a subsequent in situ chromosomal, DNA and/or RNA analysis.

As is shown in FIGS. 1-6 and is described in Examples 1-3 of the Examples section which follows, when immunologically-stained cells [e.g., using the HRP substrate aminoethylcarbazole (AEC)] are immersed in a solution of ammonium hydroxide (e.g., 2% in 70% ethanol), the residual AEC dye is efficiently removed and the subsequent FISH analysis results in optimized hybridization signals.

Thus, according to yet an additional aspect of the present invention there is provided a method of in situ chromosomal, DNA and/or RNA analysis by hybridization of a prestained specimen of cells or tissue. The method is effected by incubating the prestained specimen of cells or tissue in a solution containing ammonium hydroxide and thereafter incubating the prestained specimen of cells or tissue with a polynucleotide probe suitable for the in situ chromosomal, DNA or RNA analysis.

The phrase “prestained specimen” refers to any specimen containing cells or tissues which are stained using an immunological, cytological or activity staining. Examples of specimens which can be used according to the method of the present invention include, but are not limited to, plasma, serum, spinal fluid, lymph fluid, the external sections of the skin, respiratory, intestinal, and genitourinary tracts, tears, saliva, sputum, milk, blood cells, various tissue sections obtained from tumors, neuronal tissue, organs, embryonic cells, stem cells, cord blood, transcervical cells and samples of in vivo cell culture constituents.

As is mentioned hereinabove, immunological staining can be effected using an antibody which is directly conjugated to a dye (e.g., a fluorescent dye, a radioactive label) or via a conjugated enzyme.

According to presently preferred embodiments of the present invention, the immunological staining method of the present invention utilizes antibodies which are directly or indirectly conjugated to an enzyme capable of producing a colorimetric reaction such as horseradish peroxidase (HRP) or alkaline phosphatase. Such calorimetric reactions can utilize a chromogenic substrate such as AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate (Avivi C., et al., 1994, J Histochem Cytochem. 1994 April; 42(4):551-4) for alkaline phosphatase enzyme and Nova Red, diaminobenzidine (DAB), Vector(R) SG substrate, luminol-based chemiluminescent substrate for the peroxidase enzyme. These enzymatic substrates are commercially available from Sigma (St Louis, Mo., USA), Molecular Probes Inc. (Eugene, Oreg., USA), Vector Laboratories Inc. (Burlingame, Calif., USA), Zymed Laboratories Inc. (San Francisco, Calif., USA), Dako Cytomation (Denmark).

As is mentioned above, such a chromogenic substrate can be also utilized by an activity staining using e.g., endogenous alkaline phosphates and Fast red as a substrate.

The cytological staining used by the present invention can be any known cytological staining known in the art such as May-Grünwald-Giemsa, Giemsa, Papanicolau, Hematoxylin-Eosin and Hematoxylin. According to a presently preferred embodiment, the cytological staining used by the present invention is Hematoxylin.

The term “incubating” refers to dipping, immersing, washing or rinsing in the presence of ammonium hydroxide.

A solution containing ammonium hydroxide can be prepared using methods known in the arts using various diluents such as ethanol and methanol. According to preferred embodiments of the present invention the ammonium hydroxide used by the method according to this aspect of the present invention is diluted in ethanol, preferably in a solution of 70% ethanol.

The concentration of ammonium hydroxide used by the method according to this aspect of the present invention depends on the type of cells and the dye-to-cell ratio. For example, removal of AEC from HLA-G-stained cells requires incubation of cells in the presence of about 2% of ammonium hydroxide. Preferably, the concentration ammonium hydroxide used is at least 0.1%, more preferably, at least 0.2%, more preferably, at least 0.3%, more preferably, at least 0.4%, more preferably, at least 0.5%, more preferably, at least 0.6%, more preferably, at least 0.7%, more preferably, at least 0.8%, more preferably, at least 0.9%, more preferably, at least 1%, more preferably, between 0.1% to 30%, more preferably, between 0.1% to 20%, more preferably, between 0.1% to 15%, more preferably, between 0.1% to 10%, more preferably, between 0.1% to 9%, more preferably, between 0.1% to 8%, more preferably, between 0.1% to 7%, more preferably, between 0.1% to 6%, more preferably, between 0.1% to 5%, more preferably, between 1.5% to 5%, more preferably, between 1.5% to 4%, more preferably, between 2% to 3%, more preferably, between 2% to 2.5%, even more preferably, about 2%.

In addition, the incubation time in the presence of ammonium hydroxide depends on the concentration of ammonium hydroxide used as well as the type of cells and the dye-to-cell ratio. Such incubation time can vary from a few seconds to about 1-3 hours. For example, HLA-G stained trophoblasts can be incubated in the presence of 2% ammonium hydroxide for 45 minutes (see Example 1 of the Examples section which follows). It will be appreciated that such an incubation time can vary when different specimens are assayed and those of skills in the art are capable of adjusting the concentration of ammonium hydroxide as well as the incubation time to optimize the staining results.

According to a preferred embodiment of this aspect of the present invention incubating is effected for a time period of at least 2 seconds, more preferably, more preferably, for at least 5 seconds, more preferably, for at least 10 seconds, more preferably, for at least 20 seconds, more preferably, for at least for at least 30 seconds, more preferably, for at least 45 seconds, more preferably, for at least 1 minute, more preferably, between 1-180 minutes, more preferably, between 1-150 minutes, more preferably, between 1-120 minutes, more preferably, between 1-90 minutes, more preferably, between 1-75 minutes, more preferably, between 1-60 minutes, more preferably, between 5-60 minutes, more preferably, between 15-60 minutes, more preferably, between 25-60 minutes, more preferably, between 30-60 minutes, more preferably, between 35-55 minutes, more preferably, between 40-50 minutes, even more preferably, between 45-50 minutes.

Preferably, prior to ammonium hydroxide treatment and following the first staining of the specimens (e.g., using an immunological stain), the specimens are washed in water for about 5-10 minutes. Likewise, following ammonium hydroxide treatment the specimens are washed in water, following which they are preferably dehydrated in increasing ethanol concentrations, e.g. 70% and 100% ethanol, 5 minutes each.

In addition, following dehydration, the specimens are preferably fixed. Various methods of fixation are known in the arts and can be used along with the method of the present invention. These include, for example, fixation in the presence of methanol-acetic acid fixer (at a 3:1 ratio, respectively). Such fixation can be effected for 5 minutes at room temperature or preferably for 30 minutes in the presence of pre-chilled (at a temperature of 4° C.) methanol-acetic acid fixer. Following fixation, the specimens are preferably dried at room temperature.

As is mentioned hereinabove, following treatment with ammonium hydroxide the prestained specimen of cells or tissue is incubated with a polynucleotide probe suitable for the in situ chromosomal, DNA or RNA analysis.

As used herein the phrase “polynucleotide probe” refers to any polynucleotide which can be used to hybridize to a target nucleic acid sequence present in the specimen of the present invention. Such a polynucleotide probe can be, for example, an RNA, DNA, oligonucleotide (DNA, RNA PNA), a whole or a portion of chromosomal DNA (of a single chromosome, or of more than one chromosome) and/or a whole or a portion of a genomic DNA.

It will be appreciated that the method according to this aspect of the present invention can be used in a variety of applications which utilize double staining, e.g., cytological staining followed by RNA in situ hybridization, cytological staining followed by FISH analysis, cytological staining followed by in situ DNA analysis (e.g., PRINS, CGH), immunological staining followed by RNA in situ hybridization, immunological staining followed by FISH analysis, immunological staining followed by in situ DNA analysis (e.g., PRINS, CGH), activity staining followed by FISH analysis, activity staining followed by RNA in situ hybridization, and RNA in situ hybridization in situ DNA analysis (e.g., PRINS, CGH).

For example, for FISH analysis, following fixation and dehydration, the specimens are washed for 10-15 minutes (preferably 15 minutes) in a pre-warm solution of 2×SSC (at 37° C.), following which the specimens are fixed for 15 minutes in 0.9% formaldehyde in PBS, washed in PBS, digested for 14 minutes at 37° C. in a solution of 0.15% Pepsin, washed for 5 minutes in PBS, dehydrated in 70%, 85% and 100% ethanol (1 minute in each ethanol solution) and dried for 2 minutes over a heated plates (at 45-50° C.). Once the specimens are dried the polynucleotide probe can be added and the FISH analysis is continued according to manufacturer's instructions.

It will be appreciated that FISH analysis can be optimized by changing the denaturation conditions of the HYBrite apparatus (Abbott). Thus, denaturation of the polynucleotide probe is preferably effected for 4 or 5 minutes at 71° C. In addition, although the manufacturer's (Abbott) recommended hybridization period at 37° C. is 48 hours, similar results can be obtained if hybridization lasts for 60 hours. Moreover, to increase specificity, following hybridization the specimens are preferably washed for 2 minutes with 0.3% NP-40 in 0.4×SSC at 71° C.

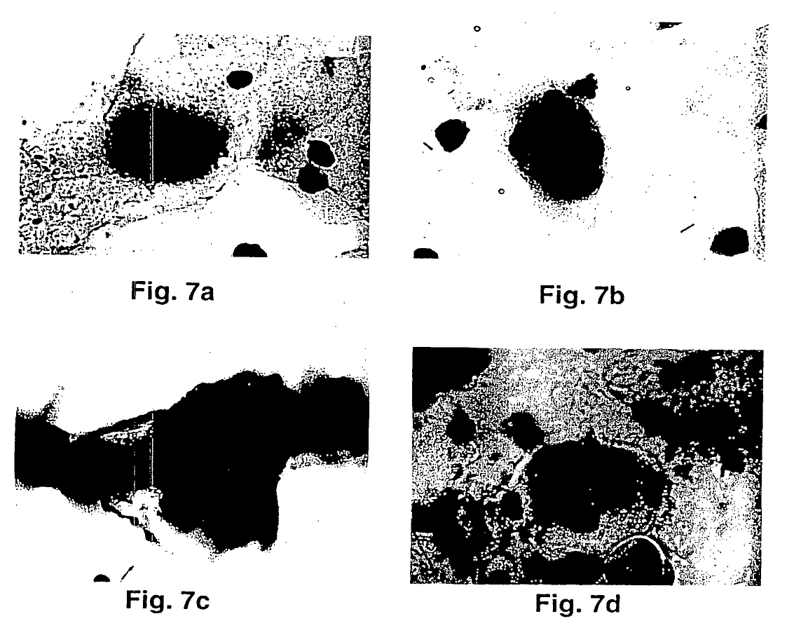

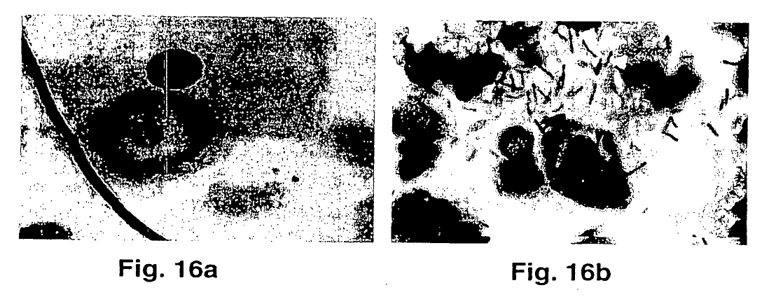

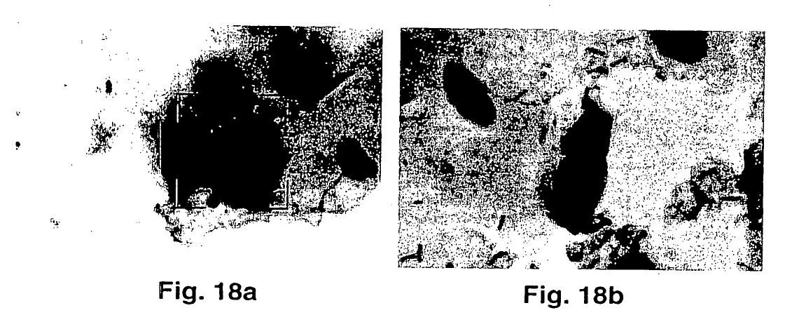

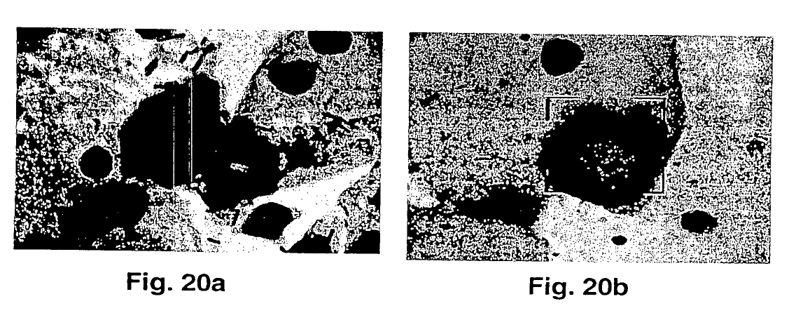

As is shown in FIGS. 7-20 and Tables 4 and 5 below, and is described in Example 4 of the Examples section which follows, the present inventors have uncovered that trophoblast cells which were positively immuno-stained with a trophoblast-specific antibody (e.g., HLA-G) exhibit distinct morphological features which are clearly distinguishable from those of non-embryonic cells which were falsely stained with the same antibody. Thus, the present inventors have provided, for the first time, a defined set of criteria of morphological features characterizing embryonic (Table 4, Example 4) and non-embryonic (Table 5, Example 4) cells present in transcervical specimens.

It will be appreciated that such morphological criteria could not have been defined prior to the teachings of the present invention which enable the determination of fetal chromosomes in transcervical specimens. For example, such morphological features were defined in cells which were positively stained with a trophoblast-specific antibody and which exhibited abnormal chromosomes or male gender. On the other hand, the morphological criteria of the non-fetal cells (as listed in Table 5) were determined using maternal cells which were falsely stained with a trophoblast-specific antibody but exhibited the maternal karyotype (e.g., the XX chromosomes) in a confirmed case of a pregnancy with a male fetus.

Thus, according to yet an additional aspect of the present invention, there is provided a method of identifying an embryonic cell in a mixed cell population. The method is effected by identifying a cell exhibiting a nucleus/cytoplasm ratio of at least 1:1 and at least a variably condensed chromatin, thereby identifying the embryonic cell in the mixed cell population.

As used herein the term “identifying” refers to establishing the identity and/or determining the presence of an embryonic cell.

The embryonic cell of the present invention can be any cell which is derived from the embryonic part of the placenta. Non-limiting examples of such cells include the villi or extravillous trophoblast cells which are shed from the placenta into the maternal blood, uterus or uterine cervix.

The phrase “mixed cell population” as used herein refers to the embryonic cells which are mixed with non-embryonic cells. Non-limiting examples of mixed cell population which can be used along with the method of the present invention include embryonic trophoblast cells with transcervical, epithelial, intrauterine or maternal blood cells.

As used herein the phrase “nucleus/cytoplasm ratio” refers to the relative area in a single plane occupied by the nucleus or of several nuclei belonging to a single cell with respect to the area occupied by the cytoplasm. As used herein the phrase “a single plane” refers to the viewing plane when an axioplan microscope is used, or to one selected plane when a multiplane or confocal microscopes are used. For example, if the area occupied by the nucleus (or all nuclei) in a single plane equals that of the cytoplasm such a ratio is considered 1:1. On the other hand, if the area occupied by the nucleus (or all nuclei) in a single plane is larger than that occupied by the cytoplasm, the ratio is presented in relative numbers such as 2:1, 3:2, 3:1, and the like. Similarly, in cases where the area occupied by the cytoplasm in a single plane is larger than that occupied by the nucleus (or all nuclei) such a ratio can be 1:2, 1:3, 2:3, 3:4 and the like.

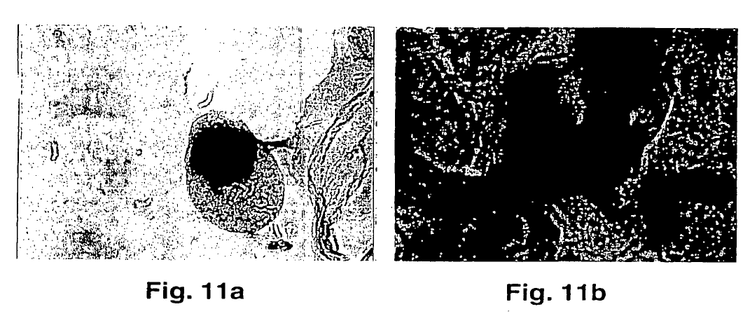

Preferably, the area occupied by the nucleus of the embryonic cell of the present invention is at least 50%, more preferably at least 55%, more preferably, at least 60%, more preferably, more preferably, at least 65%, more preferably, at least 70%, more preferably, more preferably, at least 75%, even more preferably, at least 80% of the cell area. For example, in extravillous trophoblast type II and III (FIGS. 11a-b and 12a-b, respectively), the nucleus/cytoplasm ratio is at least 2:1, respectively, i.e., the nucleus occupies about 80% of the cell area.

As used herein, the phrase “condensed chromatin” refers to the state or degree of chromatin (i.e., the DNA and histones) condensation. The chromatin is condensed to chromosomes during cell division, i.e., mitosis and meiosis. It will be appreciated that various methods can be used to determine the level of chromatin condensation, including the use of various dyes capable of attaching the nuclear (chromatin) component (e.g., DNA, proteins). Non-limiting examples of such dyes include Hematoxylin, Giemsa and DAPI. The degree of chromatin condensation is therefore determined following the appropriate nuclear staining and using a light or a fluorescent microscope.

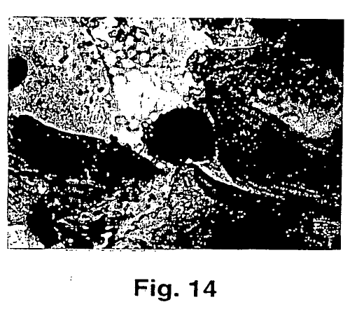

The phrase “at least variably condensed chromatin” refers to a cell in which at least a portion of the chromatin exhibits a relatively high degree of condensation, similar to that of a cell during cell division (e.g., the cell shown in FIG. 14). It will be appreciated that in a single cell a portion of the chromatin can be condensed while another portion can be relatively uncondensed (as compared to the chromatin of a cell during cell division) and those of skills in the art are capable of determining the degree of chromatin condensation in a cell.

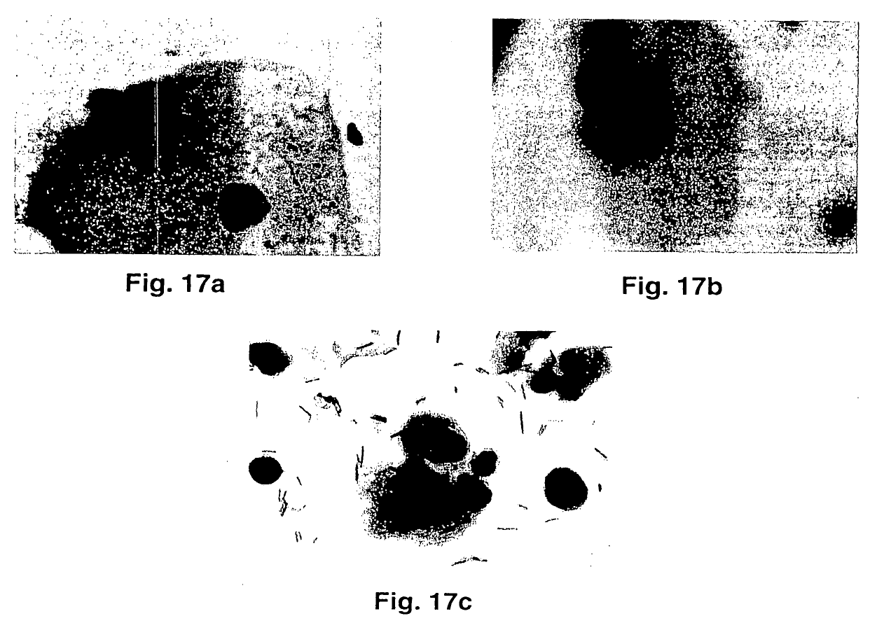

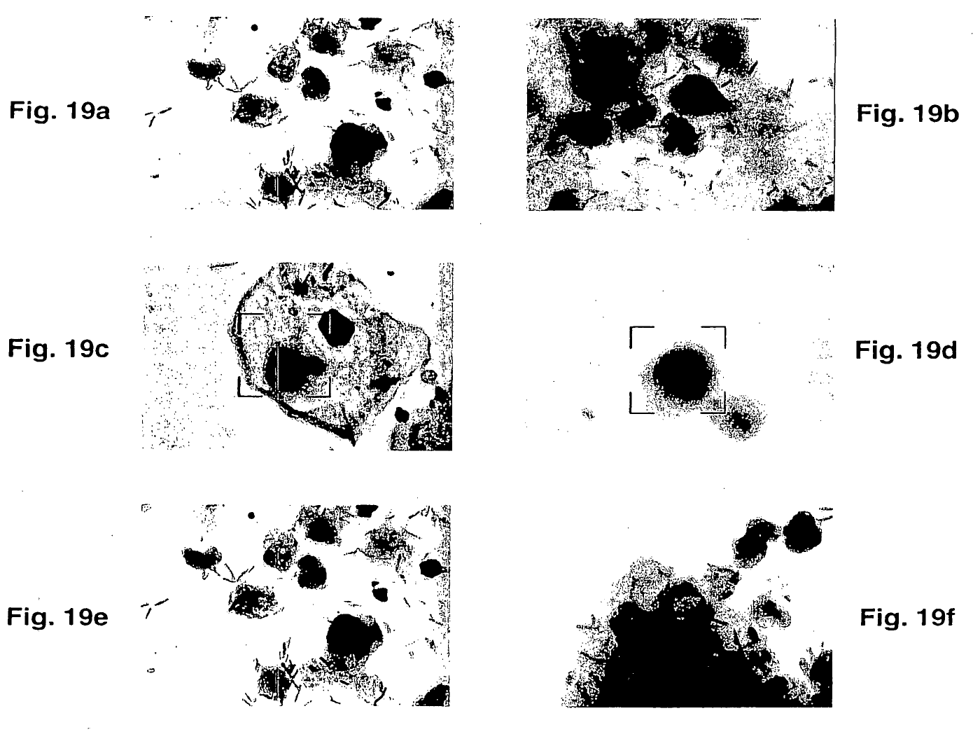

As is shown in FIGS. 7-14 and is described in Table 4 and Example 4 of the Examples section which follows, the chromatin of the embryonic cells of the present invention is variably or homogenously (i.e., uniformly) condensed. In contrast, the chromatin of some non-fetal cells which are present in the mixed cell population of the present invention and which exhibit a nucleus to cytoplasm ratio of at least 1:1, is uncondensed and thus is faintly stained with a nuclear stain (e.g., Hematoxylin). Non-limiting examples of such cells are the non-fetal “false cells” type II (FIG. 17c, Table 5 below) and type IV (FIG. 19b, Table 5 below).

According to preferred embodiments of the present invention, the method according to this aspect of the present invention further comprising evaluating at least one morphological criterion selected from the group consisting of nuclei multiplicity, nuclei arrangement, nucleus shape, cytoplasm condensation, cytoplasm shape, and cytoplasm/nucleus orientation.

As used herein the term “morphological” relates to the form and structure of the embryonic cells or component thereof, e.g., nucleus, cytoplasm, and the like.

Following are the definitions of the morphological criteria used by the present invention:

(i) Nuclei multiplicity—The number of nuclei present in a single cell. A cell according to this aspect of the present invention is defined by the presence of at least one nucleus, a cytoplasm and a single cell membrane.

(ii) Nuclei arrangement—refers to cells having more than one nucleus. In such cells, the relative arrangement of each nucleus with respect to the other nuclei present in the same cell can be random, i.e., without any specific geometric alignment, or can be aligned along a geometric structure seen on a single plane, such as a line or a circle.

(iii) Nucleus shape—The shape of the nucleus or several nuclei present in a cell can be round, egg-shape, oval, horseshoe, square-like shape, or can be amorphy, i.e., without any defined shape.

(iv) Cytoplasm condensation—The state of condensation of the cytoplasm of a single cell can be homogenously or variably condensed. Such a condensation is usually viewed using a light or fluorescent microscope following the appropriate staining (e.g., Hematoxylin, Giemsa) and those of skills in the art of histology are capable of determining the state of the cytoplasm condensation.

(v) Cytoplasm shape—The shape of the cytoplasm present in a cell can be round, fluffy or amorphy, with a well-defined or un-defined cell border (i.e., cell membrane).

(vi) Cytoplasm/nucleus orientation—The cytoplasm as seen in a single plane can be such that encompasses (i.e., surrounds) part of the nucleus or cell nuclei (i.e., 10-50%) or most of the nucleus or all nuclei (i.e., 50-100%) present in the cell. In addition, in certain cases, most of the cytoplasm area is located basal to (i.e., on one side of) the cell nucleus or cell nuclei.

(vii) Cell clump—More than two cells which appear in a cluster such that each cell of the cluster is in tight contact with at least one of the neighboring cells.

Following is a non-limiting list of examples of embryonic cells which can be identified according to the teachings of this aspect of the present invention.

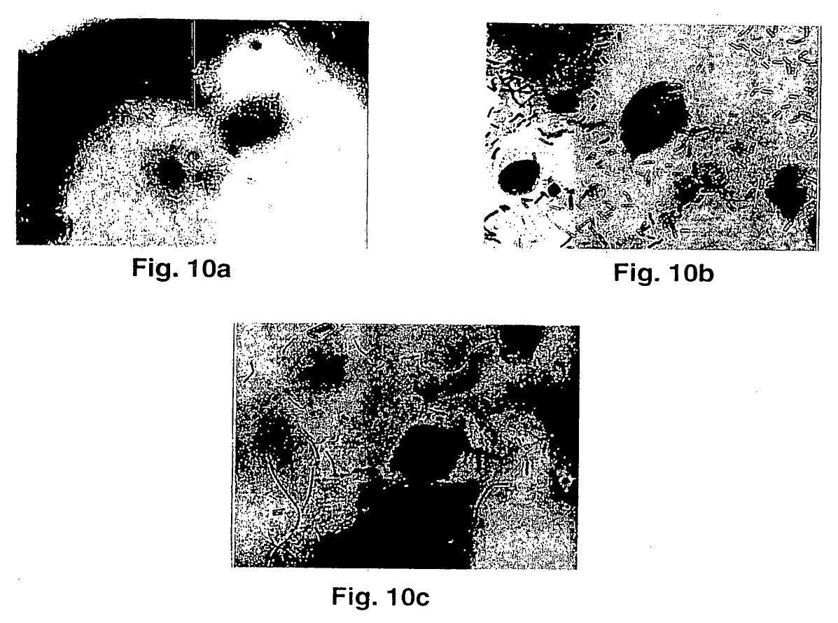

Extravillous trophoblast type I—Such cells exhibit an egg-shape, round or amorphy nucleus with variable chromatin condensation and a homogenously condensed cytoplasm which encompasses 50-85% of the nucleus. Non-limiting examples of type I extravillous trophoblasts are shown in FIGS. 10a-c.

Extravillous trophoblast type II—Such cells exhibit two nuclei in a single cell, each having an egg, round, or amorphy shape with homogenously condensed chromatin, a ratio of at least 2:1 between the total area occupied by the cell nuclei and the cytoplasm and a homogenously condensed cytoplasm which encompasses 50-85% of the total cell nuclei. Non-limiting examples of type II extravillous trophoblasts are shown in FIGS. 11a-b.

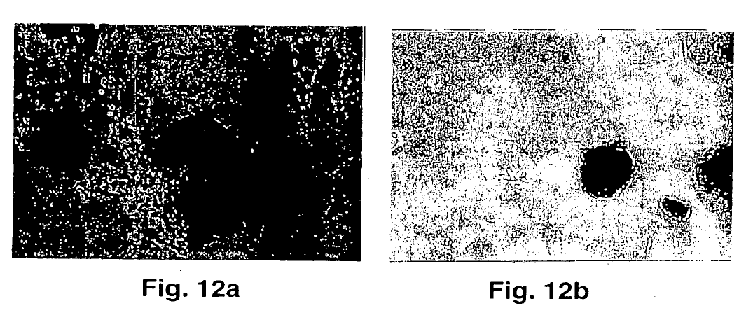

Extravillous trophoblast type III—Such cells exhibit a single nucleus having a round or egg-shape and homogenously condensed chromatin, a nucleus to cytoplasm ratio of at least 2:1, respectively and a homogenously condensed cytoplasm which is located basal to the nucleus and encompasses about 50% of the nucleus. Non-limiting examples of type III extravillous trophoblasts are shown in FIGS. 12a-b.

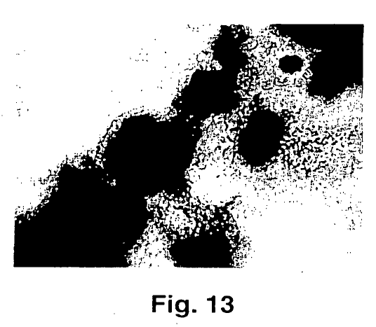

Extravillous trophoblast type IV—Such cells exhibit a horseshoe-shape and homogenously condensed chromatin, a nucleus to cytoplasm ratio of about 1:1 and a fluffy-shaped, homogenously condensed cytoplasm which encompasses 50-80% of the nucleus. A non-limiting example of type IV extravillous trophoblast is shown in FIG. 13.

Extravillous trophoblast type V—Such cells exhibit two nuclei, the chromatin in each nucleus is homogenously condensed, a nucleus to cytoplasm ratio of about 1:1, a well defined cytoplasm with variable condensation, which encompasses both nuclei. A non-limiting example of type V extravillous trophoblasts is shown in FIG. 14.

Extravillous trophoblast clump type I—Such cells appear in a cell clump, each cell clump includes more than two nuclei, each nucleus of the nuclei is arranged in a random mode with respect to the other nuclei in the cell clump, each nucleus having a round, egg or amorphy shape with variable condensation, and a fluffy-shaped and homogenously condensed cytoplasm. Non-limiting examples of extravillous trophoblast clump type I are shown in FIGS. 7a-d.

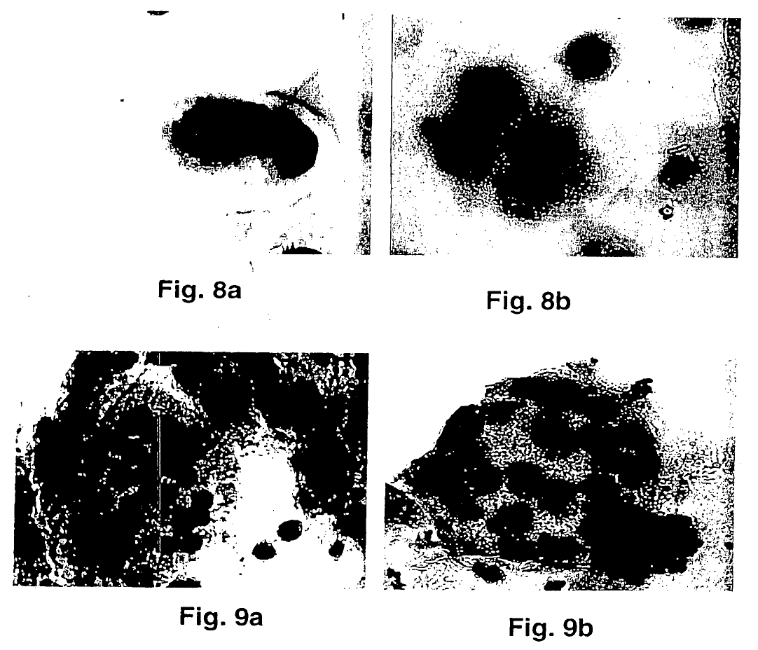

Extravillous trophoblast clump type II—Such cells appear in a cell clump, each cell clump includes more two nuclei which are arranged in a row with respect to each other, each nucleus is variably shaped and homogenously condensed, and the cytoplasm has a fluffy-shape with variable condensation. Non-limiting examples of extravillous trophoblast clump type II are shown in FIGS. 8a-b.

Syncytiotrophoblast—Such cells exhibit more than 10 nuclei in a single cell (i.e., with a single cytoplasm which is common to all nuclei), with a random arrangement of each nucleus with respect to the other nuclei of the same cell, each nucleus with a variable shape and homogenous condensation. The cytoplasm, which is common to all nuclei has a fluffy-shape, variable condensation and a well-defined cytoplasm border. Non-limiting examples of syncytiotrophoblasts are shown in FIGS. 9a-b.

The identification of embryonic cells according to the method of the present invention is preferably effected following subjecting the mixed cell population to a staining. According to preferred embodiments of the present invention such staining can be a cytological staining (e.g., May-Grünwald-Giemsa, Giemsa, Papanicolau, Hematoxylin, and Hematoxylin-Eosin), an activity staining (e.g., using a chromogenic substrate as described hereinabove) and/or an immunological staining (e.g., using an antibody directed against a trophoblast-specific antigen as described hereinabove).

Thus, the method according to this aspect of the present invention can be used to identify and classify embryonic cells (trophoblasts) from various mixed cell populations such as transcervical cells and cells derived from peripheral blood of pregnant women. In addition, the classification criteria which are provided here for the first time, can be used in prenatal diagnosis using in situ chromosomal and/or DNA analysis in a non-invasive method as described hereinabove. Moreover, since the present inventors provide definite guidelines for the identification of trophoblast cells in transcervical specimens, genetic analysis performed on such specimens is likely to result in accurate diagnosis of fetal gender and/or chromosomal abnormalities, with extremely low false positive analysis.

It is expected that during the life of this patent many relevant staining and isolating methods will be developed and the scope of the terms staining and isolating is intended to include all such new technologies a priori.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); ìNucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); In Situ Hybridization Protocolsî, Choo, K. H. A., Ed. Humana Press, Totowa, N.J. (1994); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Determination of Fetal Fish Pattern from Extravillous Trophoblast Cells Obtained from Transcervical Specimens

Transcervical cells obtained from pregnant women between 6^{th} and 15^{th} week of gestation were analyzed using immunohistochemical staining followed by FISH analysis, as follows.

Materials and Experimental Methods

Study subjects—Pregnant women between 6^{th} and 15^{th} week of gestation, which were either scheduled to undergo a pregnancy termination or were invited for a routine check-up of an ongoing pregnancy, were enrolled in the study after giving their informed consent.

Sampling of transcervical cells—A Pap smear cytobrush (MedScand-AB, Malmö, Sweden) was inserted through the external os to a maximum depth of 2 cm (the brush's length), and removed while rotating it a full turn (i.e., 360°). In order to remove the transcervical cells caught on the brush, the brush was shaken into a test tube containing 2-3 ml of the RPMI-1640 medium (Beth Haemek, Israel) in the presence of 1% Penicillin Streptomycin antibiotic. Cytospin slides (6 slides from each transcervical specimen) were then prepared by dripping 1-3 drops of the RPMI-1640 medium containing the transcervical cells into the Cytofunnel Chamber Cytocentrifuge (Thermo-Shandon, England). The conditions used for cytocentrifugation were dependent on the murkiness of the transcervical specimen; if the specimen contained only a few cells, the cells were first centrifuged for 5 minutes and then suspended with 1 ml of fresh RPMI-1640 medium. The cytospin slides were kept in 95% alcohol.

Immunohistochemical (IHC) staining of transcervical cells—Cytospin slides containing the transcervical cells were washed in 70% alcohol solution and dipped for 5 minutes in distilled water. All washes in PBS, including blocking reagent were performed while gently shaking the slides. The slides were then transferred into a moist chamber, washed three times with phosphate buffered-saline (PBS). To visualize the position of the cells on the microscopic slides, the borders of the transcervical specimens were marked using a Pap Pen (Zymed Laboratories Inc., San Francisco, Calif., USA). Fifty microliters of 3% hydrogen peroxide (Merck, Germany) were added to each slide for a 10-minute incubation at room temperature following which the slides were washed three times in PBS. To avoid non-specific binding of the antibody, two drops of a blocking reagent (Zymed HISTOSTAIN®-PLUS Kit, Cat No. 858943) were added to each slide for a 10-minute incubation in a moist chamber. To identify the fetal trophoblast cells in the transcervical sample, 50 μl of an HLA-G antibody (mAb 7759, Abcam Ltd., Cambridge, UK) part of the non-classical class I major histocompatibility complex (MHC) antigen specific to extravillous trophoblast cells (Loke, Y. W. et al., 1997. Tissue Antigens 50: 135-146) diluted 1:200 in antibody diluent solution (Zymed) or 50 μl of anti human placental alkaline phosphatase antibody (PLAP Cat. No. 18-0099, Zymed) specific to the syncytiotrophoblast and/or cytotrophoblast (Leitner, K. et al., 2001. Placental alkaline phosphatase expression at the apical and basal plasma membrane in term villous trophoblasts. J. Histochemistry and Cytochemistry, 49: 1155-1164) diluted 1:200 in antibody diluent solution were added to the slides. The slides were incubated with the antibody in a moist chamber for 60 minutes, following which they were washed three times with PBS. To detect the bound primary HLA-G specific antibody, two drops of a secondary biotinylated goat anti-mouse IgG antibody (Zymed HISTOSTAIN®-PLUS Kit, Cat No. 858943) were added to each slide for a 10-minute incubation in a moist chamber. The secondary antibody was washed three times with PBS. To reveal the biotinylated secondary antibody, two drops of an horseradish peroxidase (HRP)-streptavidin conjugate (Zymed HISTOSTAIN®-PLUS Kit, Cat No. 858943) were added for a 10-minute incubation in a moist chamber, followed by three washes in PBS. Finally, to detect the HRP-conjugated streptavidin, two drops of an aminoethylcarbazole (AEC Single Solution Chromogen/Substrate, Zymed) HRP substrate were added for a 6-minute incubation in a moist chamber, followed by three washed with PBS. Counterstaining was performed by dipping the slides for 25 seconds in a 2% of Hematoxyline solution (Sigma-Aldrich Corp., St Louis, Mo., USA, Cat. No. GHS-2-32) following which the slides were washed under tap water and covered with a coverslip.

Microscopic analysis of immunohistochemical staining—Immunostained slides containing the transcervical cells were scanned using a light microscope (AX-70, Provis, Olympus, Japan) and the location of the stained cells (trophoblasts) was marked using the coordination numbers in the microscope.

Removal of antibody residual staining following immunohistochemistry and prior to FISH analysis using ammonium hydroxide—Antibody residual staining was removed using ammonium hydroxide in the presence or absence of acetic acid. Briefly, following immunohistochemistry, slides were dipped for 5-10 minutes in distilled water until the coverslips were gently removed from slides. Stained slides were then immersed in 2% ammonium hydroxide (diluted in 70% alcohol), washed for one minute in distilled water and further immersed for a few seconds in 100% acetic acid following which they were washed for one minute in distilled water. Alternatively, stained slides were immersed in 2% ammonium hydroxide (diluted in 70% alcohol) for 45 minutes at room temperature and washed for one minute in distilled water.

Pre-treatment of immunohistochemical stained slides prior to FISH analysis—Following ammonium hydroxide treatment immunohistochemical staining the slides were dipped for 5 minutes in double-distilled water, dehydrated in 70% and 100% ethanol, 5 minutes each, and fixed for 10 minutes in a methanol-acetic acid (in a 3:1 ratio, Merck) fixer solution. Slides were then dipped for 20 minutes in a warm solution (at 37° C.) of 300 mM NaCl, 30 mM NaCitrate (2×SSC) at pH 7.0-7.5. Following incubation, the excess of the 2×SSC solution was drained off and the slides were fixed for 15 minutes at room temperature in a solution of 0.9% of formaldehyde in PBS. Slides were then washed for 10 minutes in PBS and the cells were digested for 15 minutes at 37° C. in a solution of 0.15% of Pepsin (Sigma) in 0.01 N HCl. Following Pepsin digestion slides were washed for 10 minutes in PBS and were allowed to dry. To ensure a complete dehydration, the slides were dipped in a series of 70%, 85% and 100% ethanol (1 minute each), and dried in an incubator at 45-50° C.

FISH probes—FISH analysis was carried out using a two-color technique and the following directly-labeled probes (Abbott, Ill., USA):

Sex chromosomes: The CEP X green and Y orange (Abbott cat no. 5J10-51); CEP®X SpectrumGreen™/CEP® Y (μ satellite) SpectrumOrange™ (Abbott Cat. No. 5J10-51); The CEP X/Y consists of μ satellite DNA specific to the centromere region Xp11.1-q11.1 (DXZ1) directly labeled with SpectrumGreen™ and mixed with probe specific to μ satellite DNA sequences contained within the centromere region Yp11.1-q11.1 (DYZ3) directly labeled with SpectrumOrange™.

Chromosome 21: The LSI 21q22 orange labeled (Abbott cat no. 5J13-02). The LSI 21q22 probe contains unique DNA sequences complementary to the D21S259, D21S341 and D21S342 loci within the 21q22.13 to 21q22.2 region on the long arm of chromosome 21.

Chromosome 13: The LSI® 13 SpectrumGreen™ probe (Abbott Cat. No. 5J14-18) which includes the retinoblastoma locus (RB-1 13) and sequences specific to the 13q14 region of chromosome 13.

Chromosome 18: The CEP 18 green labeled (Abbott Cat No. 5J10-18); CEP® 18 (D18Z1, a satellite) Spectrum Orange™ (ABBOTT Cat No. 5J08-18). The CEP 18 probe consists of DNA sequences specific to the alpha satellite DNA (D18Z1) contained within the centromeric region (18p11.1-q11.1) of chromosome 18.

Chromosome 16: The CEP16 (Abbott Cat. No. 6J37-17) probe hybridizes to the centromere region (satellite II, D16Z3) of chromosome 16 (16q11.2). The CEP16 probe is directly labeled with the spectrum green fluorophore.

AneuVysion probe: The CEP probes for chromosome 18 (Aqua), X (green), Y (orange) and LSI probes for 13 green and 21 orange. This FDA cleared Kit (Abbott cat. # 5J37-01) includes positive and negative control slides, 20×SSC, NP-40, DAPI II counterstain and detailed package insert.

FISH analysis on immunohistochemical stained slides—Prior to hybridization, 7 μl of the LSI/WCP hybridization buffer (Abbott) were mixed with 1 μl of a directly-labeled probe (see hereinabove), 1 μl of human Cot 1 DNA (1 μg/μl) (Abbott, Cat No. 06J31-001) and 2 μl of purified double-distilled water. The probe-hybridization solution was centrifuged for 1-3 seconds and 11 μl of the probe-hybridization solution was applied on each slides, following which, the slides were immediately covered using a coverslip.

In situ hybridization was carried out in the HYBrite apparatus (Abbott Cat. No. 2J11-04) by setting the melting temperature to 70° C. and the melting time for three minutes. The hybridization was carried out for 48 hours at 37° C.

Following hybridization, slides were washed for 2 minutes at 72° C. in a solution of 0.3% NP-40 (Abbott) in 60 mM NaCl and 6 mM NaCitrate (0.4×SSC). Slides were then immerse for 1 minute in a solution of 0.1% NP-40 in 2×SSC at room temperature, following which the slides were allowed to dry in the darkness. Counterstaining was performed using 10 μl of a DAPI II counterstain (Abbott), following which the slides were covered using a coverslip.

Subjecting slides to a repeated FISH analysis—For several slides, the FISH analysis was repeated using a different set of probes. Following hybridization with the first set of FISH probes, the slides were washed for 20 minutes in 150 mM NaCl and 15 mM NaCitrate (1×SSC), following which the slides were dipped for 10 seconds in purified double-distilled water at 71° C. Slides were then dehydrated in a series of 70%, 85% and 100% ethanol, 2 minutes each, and dried in an incubator at 45-50° C. Hybridization and post-hybridization washes were performed as described hereinabove.

Microscopic evaluation of FISH results—Following FISH analysis, the trophoblast cells (i.e., HLA-G-positive cells) were identified using the marked coordinates obtained following the immunohistochemical staining and the FISH signals in such cells were viewed using a fluorescent microscope (AX-70 Provis, Olympus, Japan).

Sampling and processing of placental tissue—A piece of approximately 0.25 cm² of a biopsy placental tissue was obtained following termination of pregnancy. The placental tissue was squashed to small pieces using a scalpel, washed three times in a solution containing KCl (43 mM) and sodium citrate (20 mM) in a 1:1 ratio and incubated for 13 minutes at room temperature. The placental tissue was then fixed by adding three drops of a methanol-acetic acid (in a 3:1 ratio) fixer solution for a 3-minute incubation, following which the solution was replaced with a fresh 3 ml fixer solution for a 45-minute incubation at room temperature. To dissociate the placental tissue into cell suspension, the fixer solution was replaced with 1-2 ml of 60% acetic acid for a 10 seconds-incubation while shaken. The placental cell suspension was then placed on a slide and air-dried.

Confirmation of chromosomal FISH analysis in ongoing pregnancies—Amniocentesis and chorionic villus sampling (CVS) were used to determine chromosomal karyotype and ultrasound scans (US) were used to determine fetal gender in ongoing pregnancies.

Experimental Results

Extravillous trophoblast cells were identified among maternal transcervical cells—To identify extravillous trophoblasts, transcervical specimens were prepared from pregnant women (6-15 weeks of gestation) and the transcervical cells were subjected to immunohistochemical staining using an HLA-G antibody. As is shown in Table 1, hereinbelow, IHC staining using the HLA-G and/or PLAP antibodies was capable of identifying extravillous, syncytiotrophoblast or cytotrophoblast cells in 230 out of the 255 transcervical specimens. In 25 transcervical specimens (10% of all cases) the transcervical cells did not include trophoblast cells. In several cases, the patient was invited for a repeated transcervical sampling and the presence of trophoblasts was confirmed (not shown). As can be calculated from Table 1, hereinbelow, the average number of HLA-G-positive cells was 6.67 per transcervical specimen (including all six cytospin slides).

Extravillous trophoblast cells were subjected to FISH analysis—Following IHC staining, the slides containing the HLA-G- or PLAP-positive cells were subjected to formaldehyde and Pepsin treatments following which FISH analysis was performed using directly-labeled FISH probes. As can be calculated from the data in Table 1, hereinbelow, the average number of cells which were marked using the FISH probes was 3.44. In most cases, the FISH results were compared to the results obtained from karyotyping of cells of placental tissue (in cases of pregnancy termination) or CVS and/or amniocentesis (in cases of ongoing pregnancies). In some cases, the confirmation of the fetal gender was performed using ultrasound scans.

TABLE 1
Determination of a FISH pattern in trophoblasts of transcervical specimens

| | | No. of | No. of | | |
|---|---|---|---|---|---|
| | | IHC- | FISH- | Gender and/or | Success/Failure of |
| Case | Gest. | positive | positive | chromosomal | the transcervical |
| No. | Weeks | cells | cells | aberration | test |
| 1 | 9 | 0 | 0 | XY | − |
| 2 | 10 | 3 | 1 | XX/XXX | + |
| 3 | 12 | 8 | 3 | XX/Trisomy 21 | + |
| 4 | 9 | 4 | 0 | XXY | − |
| 5 | 10 | 9 | 1 | XX/Trisomy 21 | + |
| 6 | 10 | 10 | 8 | XX/X0 | + |
| 7 | 10 | 1 | 0 | XY | − |
| 8 | 7 | 9 | 1 | XY | + |
| 9 | 9 | 12 | 4 | XY | + |
| 10 | 8 | 1 | 0 | XX/XXX | − |
| 11 | 8.5 | 21 | 15 | XX/X0 | + |
| 12 | 9 | 4 | 1 | XY | + |
| 13 | 9.5 | 3 | 2 | XY | + |
| 14 | 7.5 | 5 | 2 | XX/Trisomy 21 | + |
| 15 | 7 | 2 | 1 | XY | + |
| 16 | 6 | 1 | 1 | XXX | False |
| 17 | 5 | 1 | 0 | XY | − |
| 18 | 6 | 1 | 0 | XY | − |
| 19 | 6 | 0 | 0 | XY | − |
| 20 | 8 | 6 | 2 | XY | + |
| 21 | 8 | 6 | 2 | XX/Trisomy 13 | False |
| 22 | 13 | 0 | 0 | Triploid (XXX) | − |
| 23 | 9 | 5 | 1 | XY | + |
| 24 | 9.5 | 4 | 3 | XY | + |
| 25 | 10.5 | 13 | 5 | Triploid (XXY) | + |
| 26 | 9 | 10 | 4 | XY | + |
| 27 | 7.5 | 10 | 2 | XY | + |
| 28 | 9 | 7 | 0 | XY/Trisomy 13 | − |
| 29 | 12 | 4 | 0 | XY | − |
| 30 | 9.5 | 11 | 1 | XY | + |
| 31 | 11 | 2 | 1 | XY | False |
| 32 | 8 | 0 | 0 | Triploid (XXY) | − |
| 33 | 10 | 1 | 1 | XY | + |
| 34 | 8.5 | 1 | 0 | XY | − |
| 35 | 10 | 7 | 2 | XY | + |
| 36 | 8 | 8 | 5 | XY | + |
| 37 | 11 | 2 | 2 | XY | + |
| 38 | 8 | 12 | 6 | XY Twins | + |
| 39 | 6 | 3 | 2 | XX/Trisomy 21 | + |
| 40 | 13 | 9 | 5 | Triploid (XXX) | + |
| 41 | 10 | 14 | 3 | XY | + |
| 42 | 12 | 31 | 17 | XY/Trisomy 18 | + |
| 43 | 8 | 9 | 7 | XX/Trisomy 21 | + |
| 44 | 9 | 1 | 1 | XY | False |
| 45 | 14 | 1 | 0 | XY | − |
| 46 | 8 | 13 | 9 | X0 | + |
| 47 | 7 | 4 | 2 | XY | + |
| 48 | 9 | 26 | 12 | XY | + |
| 49 | 12 | 3 | 0 | XY/XXY | − |
| 50 | 10 | 5 | 1 | XX/Trisomy 13 | + |
| 51 | 10 | 10 | 5 | XX/Trisomy 21 | + |
| 52 | 7 | 4 | 2 | XY | + |
| 53 | 8 | 6 | 2 | XXYY | + |
| 54 | 10 | 7 | 6 | XY/Trisomy 21 | + |
| 55 | 7 | 7 | 0 | XY | − |
| 56 | 8 | 3 | 1 | Triploid (XXX) | + |
| 57 | 8.5 | 4 | 2 | X0 | + |
| 58 | 8.5 | 18 | 7 | XY | + |
| 59 | 8 | 22 | 6 | XY | + |
| 60 | 9 | 2 | 0 | XX/Trisomy 21 | − |
| 61 | 7 | 3 | 0 | XXX | − |
| 62 | 7 | 10 | 10 | XY | + |
| 63 | 11 | 7 | 2 | X0 | + |
| 64 | 8 | 5 | 3 | XXX | + |
| 65 | 7 | 9 | 2 | XY | + |
| 66 | 9 | 4 | 2 | XY | + |
| 67 | 10 | 8 | 2 | XY | + |
| 68 | 9.5 | 2 | 1 | XY | + |
| 69 | 9 | 8 | 1 | XXX | + |
| 70 | 7.5 | 5 | 1 | XY | + |
| 71 | 8.5 | 8 | 2 | XY/Trisomy 21 | + |
| 72 | 7 | 20 | 9 | XY | + |
| 73 | 7 | 5 | 2 | XY | + |
| 74 | 10 | 5 | 1 | X0 | + |
| 75 | 9 | 15 | 2 | Triploid (XXX) | + |
| 76 | 6 | 11 | 3 | X0 | + |
| 77 | 8 | 8 | 0 | XXX | − |
| 78 | 7 | 19 | 5 | XY | + |
| 79 | 9 | 6 | 2 | X0 | + |
| 80 | 9 | 9 | 2 | XY | + |
| 81 | 6 | 2 | 1 | X0 | + |
| 82 | 11 | 4 | 1 | Triploid (XXX) | + |
| 83 | 8 | 8 | 1 | XX | + |
| 84 | 11 | 5 | 2 | XY | + |
| 85 | 10 | 2 | 0 | XX | − |
| 86 | 11 | 5 | 1 | XY | + |
| 87 | 11 | 13 | 8 | XY | + |
| 88 | 8 | 9 | 3 | XY | + |
| 89 | 8 | 17 | 2 | XY | + |
| 90 | 8 | 1 | 1 | XY | + |
| 91 | 11 | 20 | 2 | XY | + |
| 92 | 7 | 19 | 6 | XY | + |
| 93 | 8 | 10 | 5 | X0 | + |
| 94 | 8 | 15 | 7 | XY | + |
| 95 | 8 | 16 | 6 | XY | + |
| 96 | 9 | 0 | 0 | XY | − |
| 97 | 11 | 16 | 13 | XY | + |
| 98 | 10 | 7 | 1 | XY | + |
| 99 | 6 | 14 | 3 | XY | + |
| 100 | 8 | 13 | 4 | XY | + |
| 101 | 10 | 14 | 3 | XY | + |
| 102 | 9 | 11 | 3 | XY | + |
| 103 | 10 | 11 | 3 | XY | + |
| 104 | 8 | 8 | 4 | XY | + |
| 105 | 11 | 3 | 1 | XY | + |
| 106 | 9 | 6 | 2 | XY | + |
| 107 | 8 | 8 | 3 | XY | + |
| 108 | 7 | 4 | 2 | XX | + |
| 109 | 7 | 9 | 3 | X0 | + |
| 110 | 8 | 8 | 2 | XY | + |
| 111 | 9 | 18 | 3 | XY | + |
| 112 | 10 | 4 | 3 | XY | False |
| 113 | 9.5 | 14 | 7 | XY | + |
| 114 | 11 | 4 | 1 | XY | + |
| 115 | 6.5 | 13 | 3 | XX | + |
| 116 | 8 | 5 | 1 | XY | + |
| 117 | 7 | 2 | 2 | XY | + |
| 118 | 11 | 3 | 2 | XY | + |
| 119 | 11 | 4 | 2 | XX | + |
| 120 | 7 | 1 | 0 | XX | − |
| 121 | 8 | 19 | 12 | XY | + |
| 122 | 8 | 3 | 2 | XX | + |
| 123 | 7 | 4 | 1 | XX | + |
| 124 | 8 | 2 | 0 | XY | − |
| 125 | 8 | 0 | 0 | XX | − |
| 126 | 8 | 2 | 1 | XX | + |
| 127 | 8 | 3 | 1 | X0 | + |
| 128 | 9 | 3 | 1 | X0 | + |
| 129 | 8 | 0 | 0 | XY | − |
| 130 | 7 | 5 | 2 | XY | + |
| 131 | 8 | 0 | 0 | XY | − |
| 132 | 12 | 1 | 1 | XX | + |
| 133 | 7 | 18 | 10 | XY | + |
| 134 | 8 | 20 | 17 | XX | + |
| 135 | 13 | 6 | 3 | XX | + |
| 136 | 10 | 0 | 0 | XX | − |
| 137 | 7 | 0 | 0 | XY | − |
| 138 | 8 | 4 | 4 | XX | + |
| 139 | 10 | 5 | 4 | XY | + |
| 140 | 9 | 3 | 2 | X0 | + |
| 141 | 8 | 3 | 3 | XY | + |
| 142 | 6 | 6 | 5 | XY | + |
| 143 | 7 | 3 | 3 | XY | + |
| 144 | 7 | 0 | 0 | XX | − |
| 145 | 9 | 4 | 4 | XX | + |
| 146 | 10 | 1 | 1 | XY | + |
| 147 | 12 | 3 | 2 | XY | False |
| 148 | 7 | 2 | 2 | XY | + |
| 149 | 10 | 1 | 1 | X0 | + |
| 150 | 9 | 0 | 0 | XY | − |
| 151 | 11 | 0 | 0 | XX | − |
| 152 | 8 | 2 | 2 | XX | + |
| 153 | 12 | 2 | 1 | XY | + |
| 154 | 10 | 0 | 0 | XX | − |
| 155 | 11 | 2 | 2 | XY | False |
| 156 | 8 | 2 | 2 | XY | + |
| 157 | 7.5 | 4 | 2 | XY | + |
| 158 | 8 | 13 | 10 | XY | + |
| 159 | 7 | 8 | 8 | XY | + |
| 160 | 10 | 4 | 3 | XY | + |
| 161 | 7 | 8 | 6 | XXY/XY | + |
| 162 | 7 | 3 | 3 | XY | + |
| 163 | 10 | 5 | 4 | X0 | + |
| 164 | 7 | 5 | 5 | XY | + |
| 165 | 8 | 6 | 4 | XX | + |
| 166 | 11 | 36 | 5 | XX | + |
| 167 | 8 | 12 | 1 | XY | False |
| 168 | 10 | 5 | 2 | XY | + |
| 169 | 9 | 16 | 6 | XX | + |
| 170 | 12 | 14 | 4 | XY | + |
| 171 | 10 | 11 | 4 | XX | + |
| 172 | 10 | 30 | 20 | XX | + |
| 173 | 10 | 12 | 10 | XY | + |
| 174 | 12 | 18 | 0 | XX | − |
| 175 | 11 | 17 | 5 | XY | + |
| 176 | 14 | 7 | 2 | XY | False |
| 177 | 10 | 9 | 4 | XY | + |
| 178 | 12 | 2 | 2 | XY | + |
| 179 | 11 | 13 | 5 | XY | + |
| 180 | 10 | 4 | 2 | XX | + |
| 181 | 9 | 14 | 5 | XY | + |
| 182 | 10.5 | 12 | 4 | XY | + |
| 183 | 7 | 11 | 5 | XX | + |
| 184 | 11 | 3 | 2 | XX | + |
| 185 | 10 | 5 | 4 | XY | + |
| 186 | 10 | 2 | 2 | XY | + |
| 187 | 6 | 6 | 3 | XY | + |
| 188 | 10 | 7 | 4 | XY | + |
| 189 | 8 | 6 | 5 | XX | + |
| 190 | 8 | 1 | 1 | XY | + |
| 191 | 8 | 1 | 1 | XY | + |
| 192 | 9 | 1 | 1 | XY | + |
| 193 | 8 | 0 | 0 | XX | − |
| 194 | 9 | 5 | 2 | XY | + |
| 195 | 6.5 | 8 | 5 | XY | + |
| 196 | 13 | 3 | 2 | XX | + |
| 197 | 9 | 6 | 5 | XX | + |
| 198 | 9 | 8 | 4 | XY | False |
| 199 | 9.5 | 7 | 6 | XY | + |
| 200 | 15 | 15 | 10 | XY | + |
| 201 | 15 | 8 | 7 | XY/Trisomy 21 | + |
| 202 | 13.5 | 0 | 0 | XY | − |
| 203 | 15 | 0 | 0 | XX | − |
| 204 | 7 | 7 | 7 | XY | + |
| 205 | 12 | 0 | 0 | XX | − |
| 206 | 15 | 3 | 2 | XY | + |
| 207 | 10.5 | 14 | 10 | XY | + |
| 208 | 9.5 | 10 | 5 | XY | False |
| 209 | 9 | 12 | 10 | XY | + |
| 210 | 12 | 10 | 8 | X0 | + |
| 211 | 9.5 | 1 | 1 | XY | + |
| 212 | 8 | 10 | 9 | XY | + |
| 213 | 8 | 16 | 16 | XY | + |
| 214 | 12 | 10 | 8 | XX | + |
| 215 | 10.5 | 12 | 12 | XY | + |
| 216 | 9 | 3 | 2 | XY | + |
| 217 | 8 | 8 | 7 | XX | + |
| 218 | 6.5 | 10 | 10 | XX | + |
| 219 | 9 | 1 | 1 | XY | + |
| 220 | 12 | 0 | 0 | XX | − |
| 221 | 8.5 | 8 | 7 | XX | + |
| 222 | 9 | 9 | 6 | XX | + |
| 223 | 9 | 0 | 0 | XY | − |
| 224 | 8 | 13 | 13 | XY | + |
| 225 | 12 | 2 | 1 | XY | + |
| 226 | 10 | 3 | 2 | XY | False |
| 227 | 12 | 0 | 0 | XX | − |
| 228 | 9 | 0 | 0 | XY | − |
| 229 | 11 | 3 | 2 | XY | False |
| 230 | 11.5 | 7 | 7 | XY | + |
| 231 | 14.5 | 0 | 0 | XX | − |
| 232 | 7 | 12 | 12 | XY | + |
| 233 | 9.5 | 0 | 0 | XX | − |
| 234 | 12.5 | 4 | 3 | XY | + |
| 235 | 8 | 8 | 8 | XX | + |
| 236 | 8.5 | 11 | 10 | XX | + |
| 237 | 13 | 0 | 0 | XY | − |
| 238 | 9 | 10 | 9 | XY | + |
| 239 | 11 | 4 | 3 | XY | False |
| 240 | 10 | 5 | 4 | XX | + |
| 241 | 11 | 3 | 3 | XX | + |
| 242 | 7 | 6 | 6 | XY | + |
| 243 | 11.5 | 5 | 5 | XX | + |
| 244 | 11 | 9 | 8 | XY | + |
| 245 | 10 | 4 | 4 | XX | + |
| 246 | 11 | 8 | 6 | XX | False |
| 247 | 6.5 | 5 | 3 | XY/XXY | (XY) − |
| 248 | 7 | 9 | 8 | XY | + |
| 249 | 8.5 | 9 | 9 | XX | + |
| 250 | 9.5 | 5 | 5 | XY | + |
| 251 | 12.5 | 6 | 5 | XY | + |
| 252 | 7 | 5 | 5 | XX | + |
| 253 | 6.5 | 12 | 11 | XY | + |
| 254 | 8 | 10 | 5 | XX | + |
| 255 | 7.5 | 2 | 2 | XX | + |

The success (+) or failure (−) of determination of fetal FISH pattern is presented along with the number of IHC and FISH-positive cells and the determination of gender and/or chromosomal aberrations using placental biopsy, CVS or amniocentesis.
Gest. = gestation of pregnancy;
“False” = non-specific binding of the HLA-G or the PLAP antibody to maternal cells and/or residual antibody-derived signal following FISH analysis;
* = failure in the identification of a mosaicism due to small number of cells.

The identification of normal male fetuses in extravillous trophoblasts present in transcervical specimens—Slides containing transcervical cells obtained from two different pregnant women at the 7^{th} and 9^{th} week of gestation (cases 73 and 80, respectively, in Table 1, hereinabove) were subjected to HLA-G IHC staining. As is shown in FIGS. 1a and 1c, both transcervical specimens included HLA-G-positive cells (i.e., extravillous trophoblasts). In order to determine the gender of the fetuses, following IHC staining the slides were subjected to FISH analysis using the CEP X and Y probes. As is shown in FIGS. 1b and 1d, a normal FISH pattern corresponding to a male fetus was detected in each case. These results demonstrate the use of transcervical specimens in determining the FISH pattern of fetal cells.

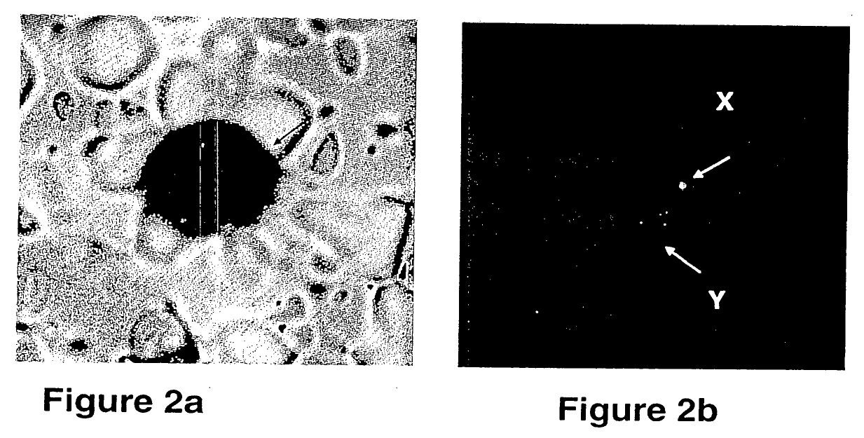

FISH pattern can be successfully determined in cytotrophoblast cells present in a transcervical specimen using the PLAP antibody—Transcervical cells obtained from a pregnant woman at the 11^{th} week of gestation were subjected to IHC staining using the anti human placental alkaline phosphatase (PLAP) antibody which is capable of identifying syncytiotrophoblast and villous cytotrophoblast cells (Miller et al., 1999 Hum. Reprod. 14: 521-531). As is shown in FIG. 2a, the PLAP antibody was capable of identifying a villous cytotrophoblast cell in a transcervical specimen. Following FISH analysis using the CEP X and Y probes the presence of a single orange and a single green signals on the villous cytotrophoblast cell (FIG. 2b, white arrow), confirmed the presence of a normal male fetus.

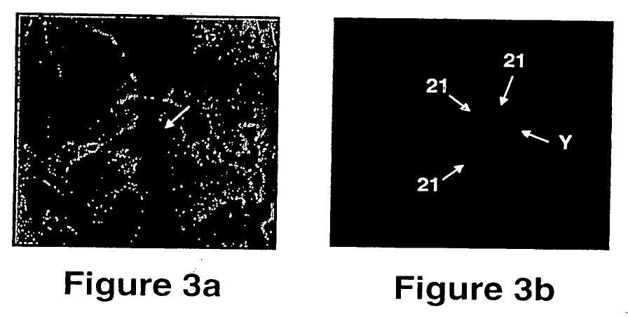

The diagnosis of Down syndrome (Trisomy 21) using extravillous trophoblasts in a transcervical specimen—Transcervical cells obtained from a pregnant woman at the 8^{th} week of gestation (case No. 71 in Table 1, hereinabove) were subjected to HLA-G IHC staining following by FISH analysis using probes specific to chromosomes Y and 21. As is shown in FIGS. 3a-b, the HLA-G-positive cell (FIG. 3a, cell marked with a white arrow) contained three orange signals and a single green signal (FIG. 3b) indicating the presence of Trisomy 21 (i.e., Down syndrome) in the extravillous trophoblast of a male fetus. These results suggest the use of identifying fetuses having Down syndrome in transcervical specimen preparations.

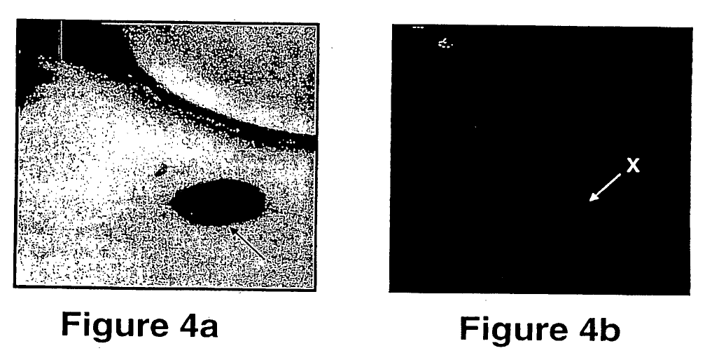

The diagnosis of Turner's syndrome (XO) using transcervical cells—Transcervical cells obtained from a pregnant woman at the 6^{th} week of gestation (case No. 76 in Table 1, hereinabove) were subjected to HLA-G IHC following by FISH analysis using probes specific to chromosomes X and Y. As is shown in FIGS. 4a-b, the presence of a single green signal following FISH analysis (FIG. 4b) in an HLA-G-positive extravillous trophoblast cell (FIG. 4a) indicated the presence of Turner's syndrome (i.e., XO) in a female fetus. These results suggest the use of identifying fetuses having Turner's syndrome in transcervical specimen preparations.

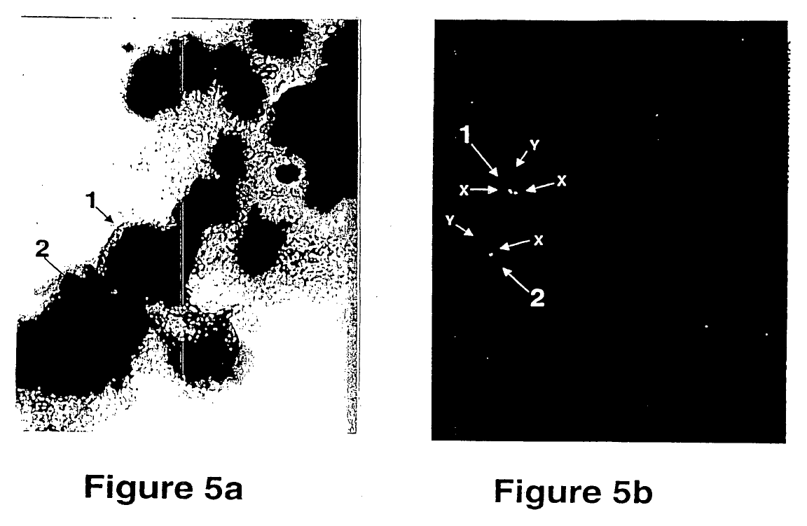

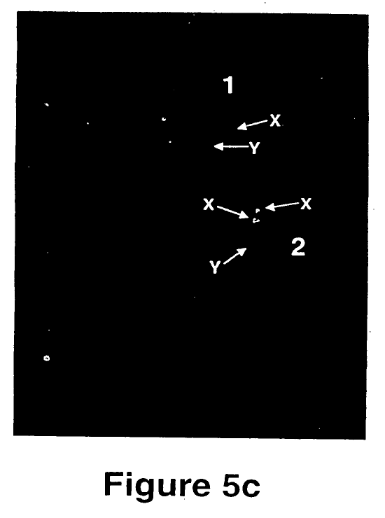

The diagnosis of Klinefelter's mosaicism using transcervical cells—Cytospin slides of transcervical specimen were prepared from a pregnant woman at the 7^{th} week of gestation (case No. 161 in Table 1, hereinabove) who was scheduled to undergo pregnancy termination. As is shown in FIGS. 5a-b, while one extravillous trophoblast cell (FIG. 5b, cell No. 1) exhibited a normal FISH pattern (i.e., a single X and a single Y chromosome), a second trophoblast cell (FIG. 5b, cell No. 2) exhibited an abnormal FISH pattern with two X chromosomes and a single Y chromosome. These results suggested the presence of Klinefelter's mosaicism in a male fetus. To verify the results, cells derived from the placental tissue obtained following termination of pregnancy, were subjected to the same FISH analysis. As is shown in FIG. 5c, the presence of Klinefelter's mosaicism was confirmed in the placental cells. Thus, chromosomal mosaicism may be detected in transcervical specimens. However, it will be appreciated that such identification may depend on the total number of trophoblast cells (i.e., IHC-positive cells) present in the transcervical specimen as well as on the percentage of the mosaic cells within the trophoblast cells.

The combined detection method of the present invention successfully determined fetal FISH pattern in 92.89% of trophoblast-containing transcervical specimens obtained from ongoing pregnancies and prior to pregnancy terminations—Table 1, hereinabove, summarizes the results of IHC and FISH analyses performed on 255 transcervical specimens which were prepared from pregnant women between the 6 to 15 week of gestation prior to pregnancy termination (cases 1-165, Table 1) or during a routine check-up (cases 166-255, Table 1, ongoing pregnancies). The overall success rate of the combined detection method of the present invention (i.e., IHC and FISH analyses) in determining the fetal FISH pattern in transcervical specimens is 76.86%. In 25/255 cases, FISH analysis was not performed due to insufficient IHC-positive cells and in 19/255 cases the FISH pattern was not determined as a result of a failure of the FISH assay (Table 1, cases marked with “-”). Among the reminder 211 cases, in 92.89% cases the fetal FISH pattern was successfully determined in trophoblast-containing transcervical specimens as confirmed by the karyotype results obtained using fetal cells of placental biopsies, amniocentesis or CVS (Table 1, cases marked with “+”). In 15/211 cases (i.e., 7.11%), the FISH analysis was performed on cells which were non-specifically interacting with the HLA-G or the PLAP antibodies, thus, leading to FISH hybridization on maternal cells (Table 1, cases marked with “False”). It will be appreciated that the percentage of cells which were non-specifically interacting with the trophoblast-specific antibodies (e.g., HLA-G or PLAP) is expected to decrease by improving the antibody preparation or the IHC assay conditions.

The combined detection method of the present invention successfully determined fetal FISH pattern in 87.34% of trophoblast-containing transcervical specimens derived from ongoing pregnancies—As can be calculated from Table 1, hereinabove, the overall success rate in determining a FISH pattern in fetal cells using transcervical specimens from ongoing pregnancies is 76.67%. Of the total of 90 transcervical specimens (cases 166-255, Table 1) obtained from pregnant women during a routine check-up (i.e., ongoing pregnancies), 11 transcervical specimens (12.2%) included IHC-negative cells. Among the reminder 79 transcervical specimens, in 8 IHC-positive samples the antibody was non-specifically interacting with maternal cells, resulting in FISH analysis of the maternal chromosomes (cases marked with “False”, Table 1), one transcervical specimen (case No. 247, Table 1) failed to identified XY/XXY mosaicism due to a small number of trophoblast cells in the sample, however, was capable of identifying the XY cells, and one transcervical specimen (case No. 174, Table 1) failed due to a technical problem with the FISH assay. Altogether, the FISH pattern was successfully determined in 69 out of 79 (87.34%) IHC-positive (i.e., trophoblast-containing) transcervical specimens.

Altogether, these results demonstrate the use of transcervical cells for the determination of a FISH pattern of fetal trophoblasts. Moreover, the results obtained from transcervical specimens in ongoing pregnancies suggest the use of transcervical cells in routine prenatal diagnosis in order to determine fetal gender and common chromosomal aberrations (e.g, trisomies, monosomies and the like). More particularly, the combined detection method of the present invention can be used in prenatal diagnosis of diseases associated with chromosomal aberrations which can be detected using FISH analysis, especially, in cases where one of the parent is a carrier of such a disease, e.g., a carrier of a Robertsonian translocation t(14; 21), a balanced reciprocal translocation t(1; 19), small microdeletion syndromes (e.g., DiGeorge, Miller-Dieker), known inversions (e.g., chromosome 7, 10) and the like.

### Example 2

### Fetal Fish Pattern can be Determined on Extra Villous Trophoblast Cells Using the HLA-G and the CHL1 Antibodies

To increase the detection rate of fetal trophoblasts in human transcervical cells, the present inventors have employed the CHL1 antibody, a new extravillous trophoblast-recognizing antibody, raised against the chorion leave from a fetal membrane (Higuchi T, et al., 2003, Mol. Hum. Reprod. 9: 359-366; Fujiwara H, et al., 1993, J. Clin. Endocrinol. Metab. 76: 956-961; Higuchi T, et al., 1999, Mol. Hum. Reprod. 5: 920-926), as follows.

Materials and Experimental Methods

CHL1 antibody—The CHL1 antibody which recognizes the melanoma cell adhesion molecule [MCAM, MeI-CAM, S-endo 1 or MUC18/CD146, Higuchi, 2003 (Supra)] was obtained from Alexis Biochemicals [Cat. No. 805-031-T100, monoclonal antibody to human CD146 (F4-35H7, S-endol; anti-MCAM)] and was diluted 1:200 prior to use on transcervical cell samples.

Immunohistochemistry and FISH analyses were performed essentially as described in Example 1, hereinabove.

Experimental Results

CHL1 antibody successfully identified extravillous trophoblast cells from transcervical cell samples—Transcervical cells were subjected to immunohistochemistry using either the HLA-G antibody or the CHL1 antibody (CD146, Alexis Biochemicals), following which stained slides were subjected to FISH analysis, essentially as described in Example 1, hereinabove. As is shown in Table 2, hereinbelow, when the CHL1 antibody was applied on transcervical specimens obtained from either ongoing pregnancies (Table 2, cases No. 140-155) or prior to pregnancy termination (Table 2, cases No. 224-241), the CHL1 antibody marked fetal trophoblast cells in 8/34 transcervical specimens. Of them, in 7 cases the antibody successfully identified fetal trophoblasts and the subsequent FISH analysis correctly determined fetal FISH pattern. In one case (case No. 239 in Table 2, hereinbelow) the CHL1 antibody non-specifically marked maternal cells instead fetal trophoblasts, resulting in false FISH results.

TABLE 2
Determination of fetal FISH pattern using HLA-G and CHL1 antibodies

| | | No. of | | | | Success/ |
|---|---|---|---|---|---|---|
| | | HLA-G | No. of | | Gender and/or | Failure of the |
| Case | Gest. | IHC-positive | CHL1 IHC- | No. of FISH-positive | chromosomal | transcervical |
| No. | Weeks | cells | positive cells | cells | aberration | test |
| 140 | 11 | 3 | 2 | 2 CHL1-positive | XX | + |
| | | | | cells | | |
| | | | | 2 HLA-G-positive | | |
| | | | | cells | | |
| 141 | 11 | 0 | 0 | 0 | XX | − |
| 142 | 11.5 | 5 | 0 | 3 | XX | + |
| 143 | 7 | 0 | 2 | 2 | XY | + |
| 144 | 9.5 | 11 | 0 | 7 | XX | + |
| 145 | 6 | 0 | 4 | 3 | XY | + |
| 146 | 7 | 2 | 0 | 2 | XX | + |
| 147 | 10 | 0 | 0 | 0 | XX | − |
| 148 | 6 | 9 | 0 | 7 | XY | FALSE |
| 149 | 8 | 6 | 0 | 4 | XX | + |
| 150 | 9 | 3 | 0 | 3 | XY | + |
| 151 | 9 | 6 | 0 | 5 | XX | + |
| 152 | 8 | 8 | 0 | 8 | XX | + |
| 153 | 7 | 0 | 3 | 2 | XY | + |
| 154 | 7 | 3 | 0 | 3 | XY | + |
| 155 | 8 | 3 | 0 | 3 | XX | + |
| 224 | 11 | 7 | 0 | 4 | XY | + |
| 225 | 7.5 | 0 | 3 | 2 | XX | + |
| 226 | 12 | 2 | 0 | 2 | XY | + |
| 227 | 6 | 0 | 0 | 0 | XX | − |
| 228 | 11 | 5 | 0 | 4 | XY | + |
| 229 | 10 | 3 | 0 | 2 | XX | + |
| 230 | 11 | 0 | 0 | 0 | XY | − |
| 231 | 7 | 8 | 0 | 5 | XY | + |
| 232 | 6 | 2 | 3 | 5 | XX | + |
| 233 | 9 | 15 | 0 | 13 | XX | + |
| 234 | 9 | 0 | 4 | 4 | XX | + |
| 235 | 7 | 5 | 0 | 4 | XX | + |
| 236 | 9 | 0 | 0 | 0 | XY | − |
| 237 | 6 | 6 | 0 | 5 | XX | + |
| 238 | 8 | 4 | 0 | 4 | XX | + |
| 239 | 8 | 0 | 3 | 2 | XY | FALSE |
| 240 | 11 | 8 | 0 | 7 | XY | + |
| 241 | 8 | 5 | 0 | 5 | XXX | + |

The success (+) or failure (−) of determination of fetal FISH pattern is presented along with the number of IHC and FISH-positive cells and the determination of gender and/or chromosomal aberrations using placental biopsy, CVS or amniocentesis.
Gest. = gestation of pregnancy;
False = non-specific binding of the HLA-G or the CHL1 antibody to maternal cells and/or residual antibody-derived signal following FISH analysis;

These results suggest the use of more than one antibody (e.g., HLA-G, PLAP and CHL1) for the detection of fetal trophoblasts in transcervical specimens.

The overall success rate of determination of fetal FISH pattern in transcervical specimens is 92.45% using HLA-G, PLAP and/or CHL1 antibodies—Table 3, hereinbelow, summarizes the results of identification of fetal gender and/or chromosomal abnormalities in 396 transcervical samples obtained from either ongoing pregnancies (cases 242-396 in Table 3) or prior to pregnancy termination (cases 1-241 in Table 3).

TABLE 3
Determination of a FISH pattern in trophoblasts of transcervical
specimens

| | | | No. of | | Success/ |
|---|---|---|---|---|---|
| | | No. of IHC- | FISH- | Gender and/or | Failure of the |
| Case | Gest. | positive | positive | chromosomal | transcervical |
| No. | Weeks | cells | cells | aberration | test |
| 1 | 9 | 0 | 0 | XY | − |
| 2 | 10 | 3 | 1 | XX/XXX | + |
| 3 | 12 | 8 | 3 | XX/Trisomy 21 | + |
| 4 | 9 | 4 | 0 | XXY | − |
| 5 | 10 | 9 | 1 | XX/Trisomy 21 | + |
| 6 | 10 | 10 | 8 | XX/X0 | + |
| 7 | 10 | 1 | 0 | XY | − |
| 8 | 7 | 9 | 1 | XY | + |
| 9 | 9 | 12 | 4 | XY | + |
| 10 | 8 | 1 | 0 | XX/XXX | − |
| 11 | 8.5 | 21 | 15 | XX/X0 | + |
| 12 | 9 | 4 | 1 | XY | + |
| 13 | 9.5 | 3 | 2 | XY | + |
| 14 | 7.5 | 5 | 2 | XX/Trisomy 21 | + |
| 15 | 7 | 2 | 1 | XY | + |
| 16 | 6 | 1 | 1 | XXX | FALSE |
| 17 | 5 | 1 | 0 | XY | − |
| 18 | 6 | 1 | 0 | XY | − |
| 19 | 6 | 0 | 0 | XY | − |
| 20 | 8 | 6 | 2 | XY | + |
| 21 | 8 | 6 | 2 | XX/Trisomy 13 | FALSE |
| 22 | 13 | 0 | 0 | Triploid (XXX) | − |
| 23 | 9 | 5 | 1 | XY | + |
| 24 | 9.5 | 4 | 3 | XY | + |
| 25 | 10.5 | 13 | 5 | Triploid (XXY) | + |
| 26 | 9 | 10 | 4 | XY | + |
| 27 | 7.5 | 10 | 2 | XY | + |
| 28 | 9 | 7 | 0 | XY/Trisomy 13 | − |
| 29 | 12 | 4 | 0 | XY | − |
| 30 | 9.5 | 11 | 1 | XY | + |
| 31 | 11 | 2 | 1 | XY | FALSE |
| 32 | 8 | 0 | 0 | Triploid (XXY) | − |
| 33 | 10 | 1 | 1 | XY | + |
| 34 | 8.5 | 1 | 0 | XY | − |
| 35 | 10 | 7 | 2 | XY | + |
| 36 | 8 | 8 | 5 | XY | + |
| 37 | 11 | 2 | 2 | XY | + |
| 38 | 8 | 12 | 6 | XY Twins | + |
| 39 | 6 | 3 | 2 | XX/Trisomy 21 | + |
| 40 | 13 | 9 | 5 | Triploid (XXX) | + |
| 41 | 10 | 14 | 3 | XY | + |
| 42 | 12 | 31 | 17 | XY/Trisomy 18 | + |
| 43 | 8 | 9 | 7 | XX/Trisomy 21 | + |
| 44 | 9 | 1 | 1 | XY | FALSE |
| 45 | 14 | 1 | 0 | XY | − |
| 46 | 8 | 13 | 9 | X0 | + |
| 47 | 7 | 4 | 2 | XY | + |
| 48 | 9 | 26 | 12 | XY | + |
| 49 | 12 | 3 | 0 | XY/XXY | − |
| 50 | 10 | 5 | 1 | XX/Trisomy 13 | + |
| 51 | 10 | 10 | 5 | XX/Trisomy 21 | + |
| 52 | 7 | 4 | 2 | XY | + |
| 53 | 8 | 6 | 2 | XXYY | + |
| 54 | 10 | 7 | 6 | XY/Trisomy 21 | + |
| 55 | 7 | 7 | 0 | XY | − |
| 56 | 8 | 3 | 1 | Triploid (XXX) | + |
| 57 | 8.5 | 4 | 2 | X0 | + |
| 58 | 8.5 | 18 | 7 | XY | + |
| 59 | 8 | 22 | 6 | XY | + |
| 60 | 9 | 2 | 0 | XX/Trisomy 21 | − |
| 61 | 7 | 3 | 0 | XXX | − |
| 62 | 7 | 10 | 10 | XY | + |
| 63 | 11 | 7 | 2 | X0 | + |
| 64 | 8 | 5 | 3 | XXX | + |
| 65 | 7 | 9 | 2 | XY | + |
| 66 | 9 | 4 | 2 | XY | FALSE |
| 67 | 10 | 8 | 2 | XY | + |
| 68 | 9.5 | 2 | 1 | XY | + |
| 69 | 9 | 8 | 1 | XXX | + |
| 70 | 7.5 | 5 | 1 | XY | + |
| 71 | 8.5 | 8 | 2 | XY/Trisomy 21 | + |
| 72 | 7 | 20 | 9 | XY | + |
| 73 | 7 | 5 | 2 | XY | + |
| 74 | 10 | 5 | 1 | X0 | + |
| 75 | 9 | 15 | 2 | Triploid (XXX) | + |
| 76 | 6 | 11 | 3 | X0 | + |
| 77 | 8 | 8 | 0 | XXX | − |
| 78 | 7 | 19 | 5 | XY | + |
| 79 | 9 | 6 | 2 | X0 | + |
| 80 | 9 | 9 | 2 | XY | + |
| 81 | 6 | 2 | 1 | X0 | + |
| 82 | 11 | 4 | 1 | Triploid (XXX) | + |
| 83 | 8 | 8 | 1 | XX | + |
| 84 | 11 | 5 | 2 | XY | + |
| 85 | 10 | 2 | 0 | XX | − |
| 86 | 11 | 5 | 1 | XY | + |
| 87 | 11 | 13 | 8 | XY | + |
| 88 | 8 | 9 | 3 | XY | FALSE |
| 89 | 8 | 17 | 2 | XY | + |
| 90 | 8 | 1 | 1 | XY | + |
| 91 | 11 | 20 | 2 | XY | + |
| 92 | 7 | 19 | 6 | XY | + |
| 93 | 8 | 10 | 5 | X0 | + |
| 94 | 8 | 15 | 7 | XY | + |
| 95 | 8 | 16 | 6 | XY | + |
| 96 | 9 | 0 | 0 | XY | − |
| 97 | 11 | 16 | 13 | XY | + |
| 98 | 10 | 7 | 1 | XY | + |
| 99 | 6 | 14 | 3 | XY | + |
| 100 | 8 | 13 | 4 | XY | + |
| 101 | 10 | 14 | 3 | XY | + |
| 102 | 9 | 11 | 3 | XY | + |
| 103 | 10 | 11 | 3 | XY | + |
| 104 | 8 | 8 | 4 | XY | + |
| 105 | 11 | 3 | 1 | XY | + |
| 106 | 9 | 6 | 2 | XY | + |
| 107 | 8 | 8 | 3 | XY | + |
| 108 | 7 | 4 | 2 | XX | + |
| 109 | 7 | 9 | 3 | X0 | + |
| 110 | 8 | 8 | 2 | XY | + |
| 111 | 9 | 18 | 3 | XY | + |
| 112 | 10 | 4 | 3 | XY | FALSE |
| 113 | 9.5 | 14 | 7 | XY | + |
| 114 | 11 | 4 | 1 | XY | + |
| 115 | 6.5 | 13 | 3 | XX | + |
| 116 | 8 | 5 | 1 | XY | + |
| 117 | 7 | 2 | 2 | XY | + |
| 118 | 11 | 3 | 2 | XY | + |
| 119 | 11 | 4 | 2 | XX | + |
| 120 | 7 | 1 | 0 | XX | − |
| 121 | 8 | 19 | 12 | XY | + |
| 122 | 8 | 3 | 2 | XX | + |
| 123 | 7 | 4 | 1 | XX | + |
| 124 | 8 | 2 | 0 | XY | − |
| 125 | 8 | 0 | 0 | XX | − |
| 126 | 8 | 2 | 1 | XX | + |
| 127 | 8 | 3 | 1 | X0 | + |
| 128 | 9 | 3 | 1 | X0 | + |
| 129 | 8 | 0 | 0 | XY | − |
| 130 | 7 | 5 | 2 | XY | + |
| 131 | 8 | 0 | 0 | XY | − |
| 132 | 12 | 1 | 1 | XX | + |
| 133 | 7 | 18 | 10 | XY | + |
| 134 | 8 | 20 | 17 | XX | + |
| 135 | 13 | 6 | 3 | XX | + |
| 136 | 10 | 0 | 0 | XX | − |
| 137 | 7 | 0 | 0 | XY | − |
| 138 | 8 | 4 | 4 | XX | + |
| 139 | 10 | 5 | 4 | XY | + |
| 140 | 9 | 3 | 2 | X0 | + |
| 141 | 8 | 3 | 3 | XY | + |
| 142 | 6 | 6 | 5 | XY | + |
| 143 | 7 | 3 | 3 | XY | + |
| 144 | 7 | 0 | 0 | XX | − |
| 145 | 9 | 4 | 4 | XX | + |
| 146 | 10 | 1 | 1 | XY | + |
| 147 | 12 | 3 | 2 | XY | FALSE |
| 148 | 7 | 2 | 2 | XY | + |
| 149 | 10 | 1 | 1 | X0 | + |
| 150 | 9 | 0 | 0 | XY | − |
| 151 | 11 | 0 | 0 | XX | − |
| 152 | 8 | 2 | 2 | XX | + |
| 153 | 12 | 2 | 1 | XY | + |
| 154 | 10 | 0 | 0 | XX | − |
| 155 | 11 | 2 | 2 | XY | FALSE |
| 156 | 8 | 2 | 2 | XY | + |
| 157 | 7.5 | 4 | 2 | XY | + |
| 158 | 8 | 13 | 10 | XY | + |
| 159 | 7 | 8 | 8 | XY | + |
| 160 | 10 | 4 | 3 | XY | + |
| 161 | 7 | 8 | 6 | XXY/XY | + |
| 162 | 7 | 3 | 3 | XY | + |
| 163 | 10 | 5 | 4 | X0 | + |
| 164 | 7 | 5 | 5 | XY | + |
| 165 | 8 | 6 | 4 | XX | + |
| 166 | 6.5 | 5 | 3 | X0/XY | + |
| 167 | 8 | 3 | 3 | XX | + |
| 168 | 6.5 | 4 | 3 | XY | + |
| 169 | 8.5 | 2 | 2 | XY | + |
| 170 | 9 | 5 | 5 | XX | + |
| 171 | 10 | 7 | 5 | XX | + |
| 172 | 8.5 | 0 | 0 | XY | − |
| 173 | 12 | 0 | 0 | XX | − |
| 174 | 6 | 4 | 3 | XY | + |
| 175 | 7 | 9 | 7 | XY | + |
| 176 | 8.5 | 6 | 5 | XY | + |
| 177 | 9 | 4 | 4 | XX | + |
| 178 | 10 | 10 | 8 | XY | + |
| 179 | 7 | 3 | 2 | XY | + |
| 180 | 12 | 5 | 5 | XX | + |
| 181 | 11 | 3 | 2 | XY | FALSE |
| 182 | 9.5 | 7 | 6 | XY | + |
| 183 | 11.5 | 0 | 0 | XX | − |
| 184 | 7 | 5 | 4 | XY | + |
| 185 | 6 | 7 | 6 | XY | + |
| 186 | 9 | 4 | 4 | XX | + |
| 187 | 11 | 0 | 0 | XX | − |
| 188 | 12 | 8 | 6 | XY | + |
| 189 | 10 | 3 | 3 | XX | + |
| 190 | 8 | 4 | 4 | XX | + |
| 191 | 7 | 2 | 2 | XX | + |
| 192 | 9 | 7 | 6 | XY | + |
| 193 | 7 | 6 | 5 | XX | + |
| 194 | 10 | 3 | 2 | XX | + |
| 195 | 9 | 7 | 7 | XY | + |
| 196 | 7 | 4 | 3 | XX | + |
| 197 | 10 | 0 | 0 | XY | − |
| 198 | 8.5 | 9 | 6 | XY | + |
| 199 | 9 | 2 | 2 | XY | + |
| 200 | 10 | 0 | 0 | XY | − |
| 201 | 7 | 10 | 8 | XY | + |
| 202 | 10 | 5 | 5 | XX | + |
| 203 | 8 | 0 | 0 | XX | − |
| 204 | 8 | 5 | 3 | XX | + |
| 205 | 11 | 3 | 2 | XY | FALSE |
| 206 | 8 | 6 | 5 | XY | + |
| 207 | 8 | 4 | 3 | XX | + |
| 208 | 10 | 10 | 8 | XY | + |
| 209 | 10 | 4 | 4 | XY | + |
| 210 | 6 | 3 | 3 | XX | + |
| 211 | 9 | 0 | 0 | XY | − |
| 212 | 6 | 3 | 2 | XX | + |
| 213 | 8.5 | 5 | 4 | XX | + |
| 214 | 6 | 3 | 3 | XX | + |
| 215 | 9 | 7 | 5 | XX | + |
| 216 | 8 | 2 | 1 | XX | + |
| 217 | 11 | 9 | 7 | XY | + |
| 218 | 11.5 | 0 | 0 | XY | − |
| 219 | 7.5 | 5 | 4 | XY | + |
| 220 | 10 | 0 | 0 | XX | − |
| 221 | 8 | 4 | 2 | XY | + |
| 222 | 9 | 5 | 4 | XY | + |
| 223 | 11.5 | 0 | 0 | XX | − |
| 224 | 11 | 7 | 4 | XY | + |
| 225 | 7.5 | 3 | 2 | XX | + |
| 226 | 12 | 2 | 2 | XY | + |
| 227 | 6 | 0 | 0 | XX | − |
| 228 | 11 | 5 | 4 | XY | + |
| 229 | 10 | 3 | 2 | XX | + |
| 230 | 11 | 0 | 0 | XY | − |
| 231 | 7 | 8 | 5 | XY | + |
| 232 | 6 | 5 | 5 | XX | + |
| 233 | 9 | 15 | 13 | XX | + |
| 234 | 9 | 4 | 4 | XX | + |
| 235 | 7 | 5 | 4 | XX | + |
| 236 | 9 | 0 | 0 | XY | − |
| 237 | 6 | 6 | 5 | XX | + |
| 238 | 8 | 4 | 4 | XX | + |
| 239 | 8 | 3 | 2 | XY | FALSE |
| 240 | 11 | 8 | 7 | XY | + |
| 241 | 8 | 5 | 5 | XXX | + |
| 242 | 6 | 6 | 3 | XY | + |
| 243 | 10 | 7 | 4 | XY | + |
| 244 | 8 | 6 | 5 | XX | + |
| 245 | 8 | 1 | 1 | XY | + |
| 246 | 8 | 1 | 1 | XY | + |
| 247 | 9 | 1 | 1 | XY | + |
| 248 | 8 | 0 | 0 | XX | − |
| 249 | 9 | 5 | 2 | XY | + |
| 250 | 6.5 | 8 | 5 | XY | + |
| 251 | 13 | 3 | 2 | XX | + |
| 252 | 9 | 6 | 5 | XX | + |
| 253 | 9 | 8 | 4 | XY | FALSE |
| 254 | 9.5 | 7 | 6 | XY | + |
| 255 | 15 | 15 | 10 | XY | + |
| 256 | 15 | 8 | 7 | XY/Trisomy 21 | + |
| 257 | 13.5 | 0 | 0 | XY | − |
| 258 | 15 | 0 | 0 | XX | − |
| 259 | 7 | 7 | 7 | XY | + |
| 260 | 12 | 0 | 0 | XX | − |
| 261 | 15 | 3 | 2 | XY | + |
| 262 | 10.5 | 14 | 10 | XY | + |
| 263 | 9.5 | 10 | 5 | XY | FALSE |
| 264 | 9 | 12 | 10 | XY | + |
| 265 | 12 | 10 | 8 | X0 | + |
| 266 | 9.5 | 1 | 1 | XY | + |
| 267 | 8 | 10 | 9 | XY | + |
| 268 | 8 | 16 | 16 | XY | + |
| 269 | 12 | 10 | 8 | XX | + |
| 270 | 10.5 | 12 | 12 | XY | + |
| 271 | 9 | 3 | 2 | XY | + |
| 272 | 8 | 8 | 7 | XX | + |
| 273 | 6.5 | 10 | 10 | XX | + |
| 274 | 9 | 1 | 1 | XY | + |
| 275 | 12 | 0 | 0 | XX | − |
| 276 | 8.5 | 8 | 7 | XX | + |
| 277 | 9 | 9 | 6 | XX | + |
| 278 | 9 | 0 | 0 | XY | − |
| 279 | 8 | 13 | 13 | XY | + |
| 280 | 12 | 2 | 1 | XY | + |
| 281 | 10 | 3 | 2 | XY | FALSE |
| 282 | 12 | 0 | 0 | XX | − |
| 283 | 9 | 0 | 0 | XY | − |
| 284 | 11.5 | 7 | 7 | XY | + |
| 285 | 14.5 | 0 | 0 | XX | − |
| 286 | 7 | 12 | 12 | XY | + |
| 287 | 9.5 | 0 | 0 | XX | − |
| 288 | 12.5 | 4 | 3 | XY | + |
| 289 | 8 | 8 | 8 | XX | + |
| 290 | 8.5 | 11 | 10 | XX | + |
| 291 | 13 | 0 | 0 | XY | − |
| 292 | 9 | 10 | 9 | XY | + |
| 293 | 11 | 4 | 3 | XY | FALSE |
| 294 | 10 | 5 | 4 | XX | + |
| 295 | 11 | 3 | 3 | XX | + |
| 296 | 7 | 6 | 6 | XY | + |
| 297 | 11.5 | 5 | 5 | XX | + |
| 298 | 11 | 9 | 8 | XY | + |
| 299 | 10 | 4 | 4 | XX | + |
| 300 | 11 | 8 | 6 | XY | FALSE |
| 301 | 6.5 | 5 | 3 | XY/XXY | (XY)− |
| 302 | 7 | 9 | 8 | XY | + |
| 303 | 8.5 | 9 | 9 | XX | + |
| 304 | 9.5 | 5 | 5 | XY | + |
| 305 | 12.5 | 6 | 5 | XY | + |
| 306 | 7 | 5 | 5 | XX | + |
| 307 | 6.5 | 12 | 11 | XY | + |
| 308 | 8 | 10 | 5 | XX | + |
| 309 | 7.5 | 2 | 2 | XX | + |
| 310 | 10.5 | 4 | 4 | XY | + |
| 311 | 8.5 | 2 | 2 | XY | + |
| 312 | 7.5 | 0 | 0 | XY | − |
| 313 | 10 | 5 | 5 | XX | + |
| 314 | 8.5 | 2 | 1 | XY | FALSE |
| 315 | 12 | 0 | 0 | XY | − |
| 316 | 9 | 5 | 5 | XX | + |
| 317 | 9 | 3 | 3 | XY | + |
| 318 | 9.5 | 4 | 3 | XX | + |
| 319 | 11 | 7 | 6 | XY | + |
| 320 | 7 | 11 | 9 | XX | + |
| 321 | 7.5 | 6 | 6 | XX | + |
| 322 | 11 | 9 | 5 | XY | + |
| 323 | 9.5 | 3 | 3 | XX | + |
| 324 | 11 | 3 | 2 | XY | + |
| 325 | 9 | 6 | 6 | XX | + |
| 326 | 12.5 | 3 | 3 | XY | + |
| 327 | 9 | 0 | 0 | XX | − |
| 328 | 7.5 | 8 | 5 | XY | FALSE |
| 329 | 10 | 2 | 2 | XX | + |
| 330 | 6 | 3 | 2 | XX | + |
| 331 | 12 | 5 | 4 | XY | + |
| 332 | 13 | 0 | 0 | XX | − |
| 333 | 7 | 6 | 6 | XX | + |
| 334 | 11 | 4 | 3 | XX | + |
| 335 | 10 | 5 | 5 | XY | + |
| 336 | 9.5 | 7 | 5 | XX | + |
| 337 | 12 | 0 | 0 | XX | − |
| 338 | 9 | 5 | 4 | XY | FALSE |
| 339 | 10.5 | 8 | 7 | XX | + |
| 340 | 7 | 0 | 0 | XY | − |
| 341 | 8 | 2 | 2 | XX | FALSE |
| 342 | 10 | 3 | 2 | XY | + |
| 343 | 8.5 | 5 | 5 | XX | + |
| 344 | 10 | 6 | 4 | XX | + |
| 345 | 8 | 3 | 3 | XX | + |
| 346 | 7 | 5 | 5 | XX | + |
| 347 | 9 | 8 | 6 | XY | + |
| 348 | 8 | 4 | 2 | XX | + |
| 349 | 8 | 5 | 5 | XY | + |
| 350 | 8.5 | 3 | 2 | XX | FALSE |
| 351 | 5.5 | 10 | 8 | XX | + |
| 352 | 8 | 5 | 5 | XX | + |
| 353 | 7 | 6 | 4 | XY | + |
| 354 | 9 | 3 | 3 | XX | + |
| 355 | 7 | 4 | 4 | XY | + |
| 356 | 9 | 6 | 5 | XX | + |
| 357 | 8.5 | 2 | 2 | XX | FALSE |
| 358 | 7 | 8 | 8 | XY | + |
| 359 | 9 | 5 | 4 | XY | + |
| 360 | 12 | 0 | 0 | XX | − |
| 361 | 8 | 7 | 7 | XY | + |
| 362 | 9 | 4 | 4 | XX | + |
| 363 | 9 | 12 | 8 | XX | + |
| 364 | 10 | 8 | 5 | XX | + |
| 365 | 9 | 4 | 3 | XX | + |
| 366 | 6.5 | 9 | 5 | XY | + |
| 367 | 6 | 9 | 8 | XY | + |
| 368 | 11 | 4 | 2 | XX | FALSE |
| 369 | 8 | 5 | 5 | XX | + |
| 370 | 7.5 | 6 | 4 | XY | + |
| 371 | 7 | 9 | 5 | XX | + |
| 372 | 9 | 2 | 2 | XX | + |
| 373 | 6 | 7 | 6 | XX | + |
| 374 | 7 | 15 | 10 | XY | + |
| 375 | 6 | 8 | 7 | XX | + |
| 376 | 7 | 2 | 2 | XX | + |
| 377 | 8.5 | 0 | 0 | XY | − |
| 378 | 9 | 5 | 5 | XY | + |
| 379 | 9 | 9 | 7 | XX | + |
| 380 | 6.5 | 3 | 3 | XY | + |
| 381 | 11 | 5 | 4 | XX | + |
| 382 | 11 | 0 | 0 | XX | − |
| 383 | 11.5 | 5 | 3 | XX | + |
| 384 | 7 | 2 | 2 | XY | + |
| 385 | 9.5 | 11 | 7 | XX | + |
| 386 | 6 | 4 | 3 | XY | + |
| 387 | 7 | 2 | 2 | XX | + |
| 388 | 10 | 0 | 0 | XX | − |
| 389 | 6 | 9 | 7 | XY | FALSE |
| 390 | 8 | 6 | 4 | XX | + |
| 391 | 9 | 3 | 3 | XY | + |
| 392 | 9 | 6 | 5 | XX | + |
| 393 | 8 | 8 | 8 | XX | + |
| 394 | 7 | 3 | 2 | XY | + |
| 395 | 7 | 3 | 3 | XY | + |
| 396 | 8 | 3 | 3 | XX | + |
| 388 | 10 | 0 | 0 | XX | − |
| 389 | 6 | 9 | 7 | XY | FALSE |
| 390 | 8 | 6 | 4 | XX | + |
| 391 | 9 | 3 | 3 | XY | + |
| 392 | 9 | 6 | 5 | XX | + |
| 393 | 8 | 8 | 8 | XX | + |
| 394 | 7 | 3 | 2 | XY | + |
| 395 | 7 | 3 | 3 | XY | + |
| 396 | 8 | 3 | 3 | XX | + |

The success (+) or failure (−) of determination of fetal FISH pattern is presented along with the number of IHC and FISH-positive cells and the determination of gender and/or chromosomal aberrations using placental biopsy, CVS or amniocentesis.
Gest. = gestation of pregnancy;
“False” = non-specific binding of the HLA-G, PLAP or the CHL1 antibody to maternal cells and/or residual antibody-derived signal following FISH analysis;

Altogether, using the HLA-G, PLAP and/or CHL1 antibodies, the present inventors were capable of successfully identifying fetal trophoblast cells in 348/396 transcervical specimens. Of them, FISH analysis, successfully determined fetal gender and/or chromosomal abnormality in 306/331 (92.45%) trophoblast-containing transcervical specimens.

### Example 3

### Identification of Sycitiotrophblasts in Transcervical Specimens Using an NDOG-1 Antibody

In attempts to improve the sensitivity of trophoblast identification in transcervical specimens, and due to the fact that syncytiotrophoblasts are not stained using common trophoblast antibodies (e.g., HLA-G) the present inventors employed the mouse anti human trophoblast protein NDOG-1 antibody on transcervical specimens obtained from pregnant women.

Prior to use, the mouse anti human trophoblast protein NDOG-1 antibody (MCA277, Serotec immunological excellence, UK) was diluted 1:50 in the antibody diluent and was incubated on the transcervical specimens according to the immunohistochemistry protocol described in “Materials and Experimental Methods” of Example 1, hereinabove.

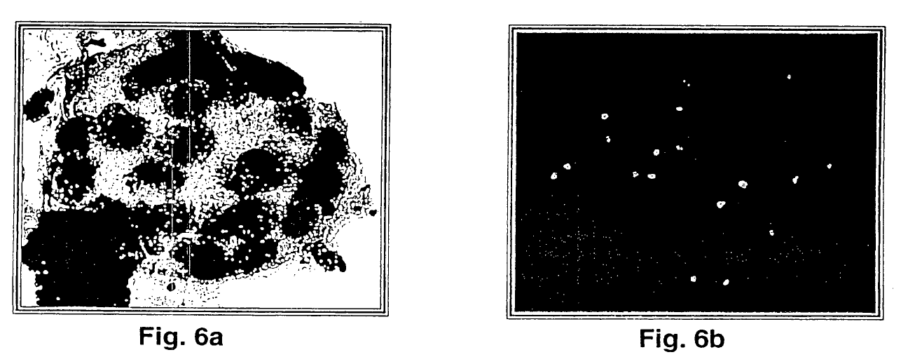

As is shown in FIGS. 6a-b, the NDOG-1 antibody specifically labeled the nuclei of the syncytiotrophoblasts present in transcervical specimens obtained from a pregnant woman at the 7^{th} week of gestation. Moreover, FISH analysis which was performed using the X and Y CEP probes according to the teachings of the present invention and the protocol described in “Materials and Experimental Methods” of Example 1, hereinabove, revealed specific signals, confirming the presence of a normal male fetus (46 XY).

Thus, these results demonstrate for the first time that syncytiotrophoblasts obtained from transcervical specimens can be used for the identification of fetal gender and/or chromosomal abnormality.

It is worth mentioning here, that from each transcervical specimen derived from one pregnant woman, at least 8 slides can be prepared for analysis as described in Example 1, and if needed (e.g., in cases where fetal cells are not found), additional 8 slides can be similarly prepared.

It is also worth mentioning here, that transcervical specimens suitable for analysis according to the methods of the present invention can be obtained from early stages of pregnancy such as from the 5^{th} week of gestation, or even earlier.

### Example 4

### Classification of Trophoblasts in Transcervical Specimens

Using the method of the present invention, the present inventors have developed a new set of criteria for classifying the cells present in transcervical specimens using various histological parameters. These criteria could have been only developed following the implementation of the method of the present invention. Thus, cells which were positively stained using the immunological staining (using e.g., HLA-G) were further evaluated for the presence of a FISH signal corresponding to the fetal karyotype (e.g., a male fetus with the X and Y signals as opposed to the maternal XX signals derived from non-embryonic cells).

Classification of Trophoblasts in Transcervical Specimens

Extravillous trophoblast clump type I—Trophoblast cells which are shed from the placenta often adhere to other trophoblast or maternal cells and form cell clumps. When such cell clumps are stained with an HLA-G antibody (e.g., MEM-G1) they are observed as multiple nuclei in various size, shape and/or condensity with an amorphic and condensed reddish cytoplasm (FIGS. 7a-d).

Extravillous trophoblast clump cells type II—Trophoblast cells which adhere to each other to form a cell clump, however, the cell nuclei are arranged in rows. The nuclei size and shape is mostly homogenous, with a condensed nuclei as reflected by the deep purple color. The cytoplasm keeps a well defined shape around the nucleus. The cytoplasm's color is homogenous red and it appears fluffy (FIGS. 8a-b).

Syncytiotrophoblast—The syncytiotrophoblast contains multiple nuclei with a common cytoplasm to the entire syncytium. Each of the nuclei in each syncytium exhibits homogenous size and shape, however, the nuclei condensity varies between different syncytium. Also note the well-defined and fluffy cytoplasm (FIGS. 9a-b).

Extravillous trophoblast Type I—Trophoblast cell with an egg-shape nucleus exhibiting variable degrees of condensation and a condensed and homogenous cytoplasm surrounding or covering some of the nucleus. The nucleus to cytoplasm ratio is 3 to 1, respectively (FIGS. 10a-c).

Extravillous trophoblast Type II—Trophoblast cell with an egg or square shape nucleus, exhibiting uniform size and degree of condensation. The nucleus to cytoplasm ratio is 4 to 1, respectively (FIGS. 11a-b).

Extravillous trophoblast Type III—Trophoblast cell with a large (about ⅔ of the cell radius) and condensed nucleus, located basal to the cytoplasm. The cytoplasm is condensed and homogenous cytoplasm and surrounds only part of the nucleus (FIGS. 12a-b).

Extravillous trophoblast Type IV—Trophoblast cell with a large (about ⅔ of the cell radius) and condensed nucleus exhibiting a horseshoe shape (not oval, not round). The cytoplasm is homogenous and fluffy cytoplasm, with no definite borders (FIG. 13).

Extravillous trophoblast Type V—A dividing trophoblast cell with two nuclei having a condensed chromatin. Total nuclei area is about 50% of total cell area. The cytoplasm is large, variably condensed cytoplasm and surrounds the entire nucleus (FIG. 14).

Table 4, hereinbelow, summarizes the various trophoblast types present in transcervical specimens.

TABLE 4
Classification of trophoblasts in transcervical specimens

| | Detecting | | |
|---|---|---|---|
| Type of Trophoblast | antibody | Nucleus Description | Cytoplasm Description |
| HLA-G - positive | HLA-G | Multiple nuclei in various size, | Amorphic with a |
| extravillous | | shape and/or condensity. | condensed reddish |
| trophoblast clump | | | color. |
| Type I | | | |
| HLA-G - positive | HLA-G | Few nuclei arranged in rows. | The cytoplasm keeps a |
| extravillous | | Nucleus size & shape mostly | well defined shape |
| trophoblast clump | | homogenous. | around the nucleus. |
| Type II | | Nuclear DNA is condensed as | The cytoplasm's color |
| | | reflected in the deep purple color. | is homogenous red and |
| | | | it appears fluffy. |
| Syncytiotrophoblast | NDOG-1 | Homogenous size & shape, | Common cytoplasm |
| | | condensity varies in different | for the entire |
| | | syncytium. | syncytium. Border well |
| | | | defined and fluffy. |
| Extravillous | HLA-G | Egg shaped nucleus, with variable | Condensed, |
| trophoblast | | chromatin condensation. | homogenous |
| Type I | | | cytoplasm that was |
| | | | colored deep red. |
| | | | Surrounds some of the |
| | | | nucleus, and in some |
| | | | parts climbs over it. |
| Extravillous | HLA-G | Egg shaped. Two nuclei with | Common cytoplasm |
| trophoblast | | condensed chromatin and uniform in | that surrounds either |
| Type II | | size. | most or all of nuclei, |
| | | | and in some parts |
| | | | climbs over it. |
| Extravillous | HLA-G | Large (about 2/3 of cell radius) and | Completes the nucleus |
| trophoblast | | condensed chromatin in the nucleus, | like a puzzle (only a |
| Type III | | located basal to cytoplasm. | part of the nucleus is |
| | | | surrounded). The |
| | | | coloring is condensed |
| | | | homogenous and |
| | | | strong. |
| Extravillous | HLA-G | Large (about 2/3 of cell radius), | Homogenous and |
| trophoblast | | condensed chromatin and horseshoe | fluffy, with no |
| Type IV | | (Not oval, not round) shaped | definitive borders. |
| | | nucleus. | |
| Extravillous | HLA-G | Dividing, condensed chromatin in | Variably condensed |
| trophoblast | | the nuclei, total nuclei area occupies | cytoplasm which |
| Type V | | about 50% of total cell area | surrounds the entire |
| | | | nucleus, with a defined |
| | | | cell border |

Table 4: Classification of trophoblast types in transcervical specimens using HLA-G and NDOG antibodies. Transcervical specimens were immunologically stained with either the HLA-G antibody or the NDOG-1 antibodies using the HRP - aminoethylcarbazole (AEC) detection method. Countersatining was performed with 2% Hematoxylin.

Altogether, these results demonstrate the accurate identification of fetal and non-fetal cells in transcervical specimens using various antibodies (e.g., HLA-G and NDOG-1). In addition, the classification criteria provided here, for the first time, can be used in prenatal diagnosis using in situ chromosomal and/or DNA analysis in a non-invasive method. Moreover, since the present inventors provide definite guidelines for the identification of trophoblast cells in transcervical specimens, genetic analysis performed on such specimens is likely to result in accurate diagnosis of fetal gender and/or chromosomal abnormalities, with extremely low false positive analysis.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

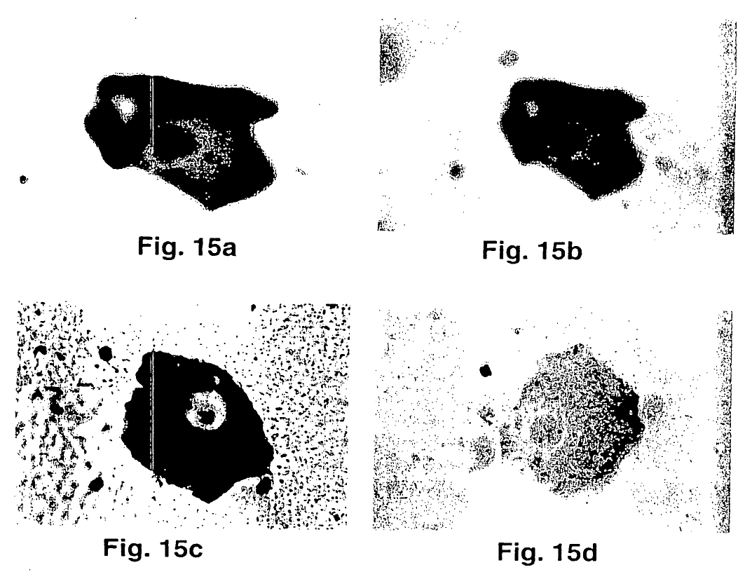

To further differentiate fetal trophoblasts from non-fetal cells which are found in a transcervical specimen, the present inventors have further characterized the morphology of the non-fetal cells following HLA-G antibody staining by using FISH analysis. Thus, cells which are now classified as non-fetal cells are those which exhibit a positive HLA-G staining but are apparently maternal cells. For example, cell which exhibit an XX FISH result (i.e., a female karyotype) but are derived from a pregnant woman with a confirmed male pregnancy. The classification of the non-fetal HLA-G positive cells present in transcervical specimens is summarized in Table 5, hereinbelow, and is further demonstrated in FIGS. 15-20.

TABLE 5
Classification of HLA-G positive immunologically stained non-fetal cells in
transcervical specimen

| | Antibody used | | |
|---|---|---|---|
| Type of cell | in preparation | Nucleus Description | Cytoplasm Description |
| Epithelial cell | HLA-G | Small (about 1/5 of cell radius) round | Large amorphous with |
| | | shape nuclei in various chromatin | various red color. |
| | | condensation. | |
| False cell- | HLA-G | One nucleus in various sizes, shapes | The cytoplasm keeps a |
| Type I | | and chromatin condensation. | well defined shape |
| | | | around the nucleus with a |
| | | | small space. The |
| | | | cytoplasm's color is |
| | | | homogenous, but |
| | | | variable red. |
| False cell- | HLA-G | Small round/oval nuclei in various | Common cytoplasm that |
| Type II | | chromatin condensation. | surrounds half or less of |
| | | | nuclei borders. In no |
| | | | parts cytoplasm climbs |
| | | | over the nuclei. |
| False cell- | HLA-G | One or two egg shaped nucleus, with | Amorphous cytoplasm |
| Type III | | variable chromatin condensation. | with no definitive |
| | | | borders colored deep red. |
| | | | Surrounds some of the |
| | | | nucleus, and in some |
| | | | parts climbs over it. |
| False cell- | HLA-G | Very small round shaped and | Very small cytoplasm |
| Type IV | | condensed chromatin in the nuclei. | (respectively to nuclei), |
| | | | colored deep red, that |
| | | | surrounds either most or |
| | | | all of nuclei, and in some |
| | | | parts climbs over it. |
| Artifact | | No nuclei | Various amorphous look |
| | | | alike cytoplasm in very |
| | | | deep red color. |

Table 5: Classification of non-fetal HLA-G positive cells present in transcervical specimens.

What is claimed is:

1. A method of determining fetal gender and/or identifying at least one chromosomal abnormality of a fetus:
(a) immunologically staining a trophoblast-containing cell sample to thereby identify at least one trophoblast cell, and;
(b) subjecting said at least one trophoblast cell to in situ chromosomal and/or DNA analysis to thereby determine fetal gender and/or identify at least one chromosomal abnormality.

2. The method of claim 1, wherein said trophoblast-containing cell sample is obtained from a cervix and/or a uterine.

3. The method of claim 1, wherein said trophoblast-containing cell sample is obtained using a method selected from the group consisting of aspiration, cytobrush, cotton wool swab, endocervical lavage and intrauterine lavage.

4. The method of claim 1, wherein said trophoblast cell sample is obtained from a pregnant woman at 6^{th} to 15^{th} week of gestation.

5. The method of claim 1, wherein said trophoblast cell sample is obtained from a pregnant woman at 5^{th} to 15^{th} week of gestation.

6. The method of claim 1, wherein said immunologically staining is effected using an antibody directed against a trophoblast specific antigen.

7. The method of claim 6, wherein said trophoblast specific antigen is selected from the group consisting of HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

8. The method of claim 1, wherein said in situ chromosomal and/or DNA analysis is effected using fluorescent in situ hybridization (FISH), primed in situ labeling (PRINS), multicolor-banding (MCB) and/or quantitative FISH (Q-FISH).

9. The method of claim 8, wherein said Q-FISH is effected using a peptide nucleic acid (PNA) oligonucleotide probe.

10. The method of claim 1, wherein said at least one chromosomal abnormality is selected from the group consisting of aneuploidy, translocation, subtelomeric rearrangement, unbalanced subtelomeric rearrangement, deletion, microdeletion, inversion, duplication, and telomere instability and/or shortening.

11. The method of claim 10, wherein said chromosomal aneuploidy is a complete and/or partial trisomy.

12. The method of claim 11, wherein said trisomy is selected from the group consisting of trisomy 21, trisomy 18, trisomy 13, trisomy 16, XXY, XYY, and XXX.

13. The method of claim 10, wherein said chromosomal aneuploidy is a complete and/or partial monosomy.

14. The method of claim 13, wherein said monosomy is selected from the group consisting of monosomy X, monosomy 21, monosomy 22, monosomy 16 and monosomy 15.

15. A method of determining fetal gender and/or identifying at least one chromosomal and/or DNA abnormality of a fetus:
(a) immunologically staining a trophoblast-containing cell sample to thereby identify at least one trophoblast cell;
(b) subjecting said at least one trophoblast cell to in situ chromosomal and/or DNA analysis to thereby obtain at least one stained trophoblast cell, and;
(c) subjecting at least one stained trophoblast cell to a genetic analysis to thereby determine fetal gender and/or identify at least one chromosomal and/or DNA abnormality.

16. The method of claim 15, further comprising a step of isolating said at least one stained trophoblast cell prior to step (c).

17. The method of claim 16, wherein said isolating said at least one stained trophoblast is effected using laser microdissection.

18. The method of claim 15, wherein said genetic analysis utilizes at least one method selected from the group consisting of comparative genome hybridization (CGH) and identification of at least one nucleic acid substitution.

19. The method of claim 15, wherein said trophoblast-containing cell sample is obtained from a cervix and/or a uterine.

20. The method of claim 15, wherein said trophoblast-containing cell sample is obtained using a method selected from the group consisting of aspiration, cytobrush, cotton wool swab, endocervical lavage and intrauterine lavage.

21. The method of claim 15, wherein said trophoblast-containing cell sample is obtained from a pregnant woman at 6^{th} to 15^{th} week of gestation.

22. The method of claim 15, wherein said trophoblast-containing cell sample is obtained from a pregnant woman at 5^{th} to 15^{th} week of gestation.

23. The method of claim 15, wherein said immunologically staining is effected using an antibody directed against a trophoblast specific antigen.

24. The method of claim 23, wherein said trophoblast specific antigen is selected from the group consisting of HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

25. The method of claim 15, wherein said in situ chromosomal and/or DNA analysis is effected using fluorescent in situ hybridization (FISH), primed in situ labeling (PRINS), multicolor-banding (MCB) and/or quantitative FISH (Q-FISH).

26. The method of claim 25, wherein said Q-FISH is effected using a peptide nucleic acid (PNA) oligonucleotide probe.

27. The method of claim 18, wherein said CGH is effected using metaphase chromosomes and/or a CGH-array.

28. The method of claim 18, wherein said identification of at least one nucleic acid substitution is effected using a method selected from the group consisting of DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

29. The method of claim 15, wherein said at least one DNA abnormality is selected from the group consisting of single nucleotide substitution, micro-deletion, micro-insertion, short deletions, short insertions, multinucleotide changes, DNA methylation and loss of imprint (LOI).

30. The method of claim 15, wherein said at least one chromosomal abnormality is selected from the group consisting of aneuploidy, translocation, subtelomeric rearrangement, unbalanced subtelomeric rearrangement, deletion, microdeletion, inversion, duplication, and telomere instability and/or shortening.

31. The method of claim 30, wherein said chromosomal aneuploidy is a complete and/or partial trisomy.

32. The method of claim 31, wherein said trisomy is selected from the group consisting of trisomy 21, trisomy 18, trisomy 13, trisomy 16, XXY, XYY, and XXX.

33. The method of claim 30, wherein said chromosomal aneuploidy is a complete and/or partial monosomy.

34. The method of claim 33, wherein said monosomy is selected from the group consisting of monosomy X, monosomy 21, monosomy 22, monosomy 16 and monosomy 15.

35. A method of determining fetal gender and/or identifying at least one chromosomal abnormality of a fetus:
(a) immunologically staining a trophoblast-containing cell sample to thereby identify at least one trophoblast cell, and;
(b) subjecting said at least one trophoblast cell to a genetic analysis to thereby determine fetal gender and/or identify at least one chromosomal abnormality.

36. The method of claim 35, further comprising a step of isolating said at least one stained trophoblast cell prior to step (b).

37. The method of claim 36, wherein said isolating said at least one trophoblast cell is effected using laser microdissection.

38. The method of claim 35, wherein said genetic analysis utilizes comparative genome hybridization (CGH).

39. The method of claim 35, wherein said trophoblast-containing cell sample is obtained from a cervix and/or a uterine.

40. The method of claim 35, wherein said trophoblast-containing cell sample is obtained using a method selected from the group consisting of aspiration, cytobrush, cotton wool swab, endocervical lavage and intrauterine lavage.

41. The method of claim 35, wherein said trophoblast-containing cell sample is obtained from a pregnant woman at 6^{th} to 15^{th} week of gestation.

42. The method of claim 35, wherein said trophoblast-containing cell sample is obtained from a pregnant woman at 5^{th} to 15^{th} week of gestation.

43. The method of claim 35, wherein said immunologically staining is effected using an antibody directed against a trophoblast specific antigen.

44. The method of claim 43, wherein said trophoblast specific antigen is selected from the group consisting of HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

45. The method of claim 38, wherein said CGH is effected using metaphase chromosomes and/or a CGH-array.

46. The method of claim 35, wherein said at least one chromosomal abnormality is selected from the group consisting of aneuploidy, deletion, microdeletion, duplication, unbalanced translocation, unbalanced inversion, unbalanced chromosomal rearrangement, and unbalanced subtelomeric rearrangement.

47. The method of claim 46, wherein said chromosomal aneuploidy is a complete and/or partial trisomy.

48. The method of claim 47, wherein said trisomy is selected from the group consisting of trisomy 21, trisomy 18, trisomy 13, trisomy 16, XXY, XYY, and XXX.

49. The method of claim 46, wherein said chromosomal aneuploidy is a complete and/or partial monosomy.

50. The method of claim 49, wherein said monosomy is selected from the group consisting of monosomy X, monosomy 21, monosomy 22, monosomy 16 and monosomy 15.

51. A method of determining fetal gender and/or identifying at least one chromosomal abnormality of a fetus, comprising sequentially subjecting a trophoblast-containing cell sample to an RNA-in situ hybridization (RNA-ISH) staining and an in situ chromosomal and/or DNA analysis to thereby determine fetal gender and/or identify at least one chromosomal abnormality.

52. The method of claim 51, wherein said trophoblast-containing cell sample is obtained from a cervix and/or a uterine.

53. The method of claim 51, wherein said trophoblast-containing cell sample is obtained using a method selected from the group consisting of aspiration, cytobrush, cotton wool swab, endocervical lavage and intrauterine lavage.

54. The method of claim 51, wherein said trophoblast-containing cell sample is obtained from a pregnant woman at 6^{th} to 15^{th} week of gestation.

55. The method of claim 51, wherein said trophoblast-containing cell sample is obtained from a pregnant woman at 5^{th} to 15^{th} week of gestation.

56. The method of claim 51, wherein said RNA-ISH staining is effected using a probe selected from the group consisting of an RNA molecule, a DNA molecule and a PNA oligonucleotide.

57. The method of claim 56, wherein said RNA molecule is an RNA oligonucleotide and/or an in vitro transcribed RNA.

58. The method of claim 56, wherein said DNA molecule is an oligonucleotide and/or a cDNA molecule.

59. The method of claim 56, wherein said probe is selected capable of identifying a trophoblast specific RNA transcript.

60. The method of claim 59, wherein said trophoblast specific RNA transcript is selected from the group consisting of H19, HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

61. The method of claim 51, wherein said in situ chromosomal and/or DNA analysis is effected using fluorescent in situ hybridization (FISH), primed in situ labeling (PRINS), multicolor-banding (MCB) and/or quantitative FISH (Q-FISH).

62. The method of claim 61, wherein said Q-FISH is effected using a peptide nucleic acid (PNA) oligonucleotide probe.

63. The method of claim 51, wherein said at least one chromosomal abnormality is selected from the group consisting of aneuploidy, translocation, subtelomeric rearrangement, unbalanced subtelomeric rearrangement, deletion, microdeletion, inversion, duplication, and telomere instability and/or shortening.

64. The method of claim 63, wherein said chromosomal aneuploidy is a complete and/or partial trisomy.

65. The method of claim 64, wherein said trisomy is selected from the group consisting of trisomy 21, trisomy 18, trisomy 13, trisomy 16, XXY, XYY, and XXX.

66. The method of claim 63, wherein said chromosomal aneuploidy is a complete and/or partial monosomy.

67. The method of claim 66, wherein said monosomy is selected from the group consisting of monosomy X, monosomy 21, monosomy 22, monosomy 16 and monosomy 15.

68. A method of determining fetal gender and/or identifying at least one chromosomal and/or DNA abnormality of a fetus, comprising:
(a) simultaneously or sequentially subjecting a trophoblast-containing cell sample to an RNA-in situ hybridization (RNA-ISH) staining and an in situ chromosomal and/or DNA analysis to thereby obtain at least one stained trophoblast cell and;
(b) subjecting said at least one stained trophoblast cell to a genetic analysis to thereby determine fetal gender and/or identify at least one chromosomal and/or DNA abnormality.

69. The method of claim 68, further comprising a step of isolating said at least one stained trophoblast cell prior to step (b).

70. The method of claim 69, wherein said isolating said at least one stained trophoblast cell is effected using laser microdissection.

71. The method of claim 68, wherein said genetic analysis utilizes at least one method selected from the group consisting of comparative genome hybridization (CGH) and identification of at least one nucleic acid substitution.

72. The method of claim 68, wherein said trophoblast-containing cell sample is obtained from a cervix and/or a uterine.

73. The method of claim 68, wherein said trophoblast-containing cell sample is obtained using a method selected from the group consisting of aspiration, cytobrush, cotton wool swab, endocervical lavage and intrauterine lavage.

74. The method of claim 68, wherein said trophoblast-containing cell sample is obtained from a pregnant woman at 6^{th} to 15^{th} week of gestation.

75. The method of claim 68, wherein said trophoblast-containing cell sample is obtained from a pregnant woman at 5^{th} to 15^{th} week of gestation.

76. The method of claim 68, wherein said RNA-ISH staining is effected using a probe selected from the group consisting of an RNA molecule, a DNA molecule and a PNA oligonucleotide.

77. The method of claim 76, wherein said RNA molecule is an RNA oligonucleotide and/or an in vitro transcribed RNA.

78. The method of claim 76, wherein said DNA molecule is an oligonucleotide and/or a cDNA molecule.

79. The method of claim 76, wherein said probe is selected capable of identifying a trophoblast specific RNA transcript.

80. The method of claim 79, wherein said trophoblast specific RNA transcript is selected from the group consisting of H19, HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

81. The method of claim 68, wherein said in situ chromosomal and/or DNA analysis is effected using fluorescent in situ hybridization (FISH), primed in situ labeling (PRINS), multicolor-banding (MCB) and/or quantitative FISH (Q-FISH).

82. The method of claim 81, wherein said Q-FISH is effected using a peptide nucleic acid (PNA) oligonucleotide probe.

83. The method of claim 68, wherein said at least one chromosomal abnormality is selected from the group consisting of aneuploidy, translocation, subtelomeric rearrangement, unbalanced subtelomeric rearrangement, deletion, microdeletion, inversion, duplication, and telomere instability and/or shortening.

84. The method of claim 83, wherein said chromosomal aneuploidy is a complete and/or partial trisomy.

85. The method of claim 84, wherein said trisomy is selected from the group consisting of trisomy 21, trisomy 18, trisomy 13, trisomy 16, XXY, XYY, and XXX.

86. The method of claim 83, wherein said chromosomal aneuploidy is a complete and/or partial monosomy.

87. The method of claim 86, wherein said monosomy is selected from the group consisting of monosomy X, monosomy 21, monosomy 22, monosomy 16 and monosomy 15.

88. The method of claim 71, wherein said CGH is effected using metaphase chromosomes and/or a CGH-array.

89. The method of claim 71, wherein said identification of at least one nucleic acid substitution is effected using a method selected from the group consisting of DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

90. The method of claim 68, wherein said at least one DNA abnormality is selected from the group consisting of single nucleotide substitution, micro-deletion, micro-insertion, short deletions, short insertions, multinucleotide changes, DNA methylation and loss of imprint (LOI).

91. A method of determining fetal gender and/or identifying at least one chromosomal and/or DNA abnormality of a fetus, comprising:
(a) subjecting a trophoblast-containing cell sample to an RNA-in situ hybridization (RNA-ISH) staining to thereby obtain at least one stained trophoblast cell, and;
(b) subjecting said at least one stained trophoblast cell to a genetic analysis to thereby determine fetal gender and/or identify at least one chromosomal and/or DNA abnormality.

92. The method of claim 91, further comprising a step of isolating said at least one stained trophoblast cell prior to step (b).

93. The method of claim 92, wherein said isolating said at least one stained trophoblast cell is effected using laser microdissection.

94. The method of claim 91, wherein said genetic analysis utilizes at least one method selected from the group consisting of comparative genome hybridization (CGH) and identification of at least one nucleic acid substitution.

95. The method of claim 91, wherein said trophoblast-containing cell sample is obtained from a cervix and/or a uterine.

96. The method of claim 91, wherein said trophoblast-containing cell sample is obtained using a method selected from the group consisting of aspiration, cytobrush, cotton wool swab, endocervical lavage and intrauterine lavage.

97. The method of claim 91, wherein said trophoblast-containing cell sample is obtained from a pregnant woman at 6^{th} to 15^{th} week of gestation.

98. The method of claim 91, wherein said trophoblast-containing cell sample is obtained from a pregnant woman at 5^{th} to 15^{th} week of gestation.

99. The method of claim 91, wherein said RNA-ISH staining is effected using a probe selected from the group consisting of an RNA molecule, a DNA molecule and a PNA oligonucleotide.

100. The method of claim 99, wherein said RNA molecule is an RNA oligonucleotide and/or an in vitro transcribed RNA.

101. The method of claim 99, wherein said DNA molecule is an oligonucleotide and/or a cDNA molecule.

102. The method of claim 99, wherein said probe is selected capable of identifying a trophoblast specific RNA transcript.

103. The method of claim 102, wherein said trophoblast specific RNA transcript is selected from the group consisting of H19, HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

104. The method of claim 91, wherein said at least one chromosomal abnormality is selected from the group consisting of aneuploidy, deletion, microdeletion, duplication, unbalanced translocation, unbalanced inversion, unbalanced chromosomal rearrangement, and unbalanced subtelomeric rearrangement.

105. The method of claim 104, wherein said chromosomal aneuploidy is a complete and/or partial trisomy.

106. The method of claim 105, wherein said trisomy is selected from the group consisting of trisomy 21, trisomy 18, trisomy 13, trisomy 16, XXY, XYY, and XXX.

107. The method of claim 104, wherein said chromosomal aneuploidy is a complete and/or partial monosomy.

108. The method of claim 107, wherein said monosomy is selected from the group consisting of monosomy X, monosomy 21, monosomy 22, monosomy 16 and monosomy 15.

109. The method of claim 94, wherein said CGH is effected using metaphase chromosomes and/or a CGH-array.

110. The method of claim 94, wherein said identification of at least one nucleic acid substitution is effected using a method selected from the group consisting of DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

111. The method of claim 91, wherein said at least one DNA abnormality is selected from the group consisting of single nucleotide substitution, micro-deletion, micro-insertion, short deletions, short insertions, multinucleotide changes, DNA methylation and loss of imprint (LOI).

112. A method of determining a paternity of a fetus, comprising:
(a) subjecting a trophoblast-containing cell sample to an RNA-in situ hybridization (RNA-ISH) staining to thereby obtain at least one stained trophoblast cell;
(b) subjecting said at least one stained trophoblast cell to a genetic analysis to thereby identify polymorphic markers of the fetus, and;
(c) comparing said identified polymorphic markers of the fetus to a set of polymorphic markers obtained from at least one potential father to thereby determine the paternity of the fetus.

113. The method of claim 112, further comprising a step of isolating said at least one stained trophoblast cell prior to step (b).

114. The method of claim 113, wherein said isolating said at least one stained trophoblast cell is effected using laser microdissection.

115. The method of claim 112, wherein said genetic analysis utilizes a method selected from the group consisting of PCR and/or PCR-RFLP.

116. The method of claim 112, wherein said genetic analysis is capable of detecting short tandem repeats, variable number of tandem repeats (VNTR) and/or minisatellites variant repeats (MVR).

117. The method of claim 112, wherein said trophoblast-containing cell sample is obtained from a cervix and/or a uterine.

118. The method of claim 112, wherein said trophoblast-containing cell sample is obtained using a method selected from the group consisting of aspiration, cytobrush, cotton wool swab, endocervical lavage and intrauterine lavage.

119. The method of claim 112, wherein said trophoblast-containing cell sample is obtained from a pregnant woman at 6^{th} to 15^{th} week of gestation.

120. The method of claim 112, wherein said trophoblast-containing cell sample is obtained from a pregnant woman at 5^{th} to 15^{th} week of gestation.

121. The method of claim 112, wherein said RNA-ISH staining is effected using a probe selected from the group consisting of an RNA molecule, a DNA molecule and a PNA oligonucleotide.

122. The method of claim 121, wherein said RNA molecule is an RNA oligonucleotide and/or an in vitro transcribed RNA.

123. The method of claim 121, wherein said DNA molecule is an oligonucleotide and/or a cDNA molecule.

124. The method of claim 121, wherein said probe is selected capable of identifying a trophoblast specific RNA transcript.

125. The method of claim 124, wherein said trophoblast specific RNA transcript is selected from the group consisting of H19, HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

126. A method of determining a paternity of a fetus, comprising:
(a) immunologically staining a trophoblast-containing cell sample to thereby identify at least one trophoblast cell, and;
(b) subjecting said at least one stained trophoblast cell to a genetic analysis to thereby identify polymorphic markers of the fetus, and;
(c) comparing said identified polymorphic markers of the fetus to a set of polymorphic markers obtained from a potential father to thereby determine the paternity of the fetus.

127. The method of claim 126, further comprising a step of isolating said at least one stained trophoblast cell prior to step (b).

128. The method of claim 127, wherein said isolating said at least one stained trophoblast cell is effected using laser microdissection.

129. The method of claim 126, wherein said genetic analysis utilizes a method selected from the group consisting of PCR and/or PCR-RFLP.

130. The method of claim 126, wherein said genetic analysis is capable of detecting short tandem repeats (STR), variable number of tandem repeats (VNTR) and/or minisatellites variant repeats (MVR).

131. The method of claim 126, wherein said trophoblast-containing cell sample is obtained from a cervix and/or a uterine.

132. The method of claim 126, wherein said trophoblast-containing cell sample is obtained using a method selected from the group consisting of aspiration, cytobrush, cotton wool swab, endocervical lavage and intrauterine lavage.

133. The method of claim 126, wherein said trophoblast-containing cell sample is obtained from a pregnant woman at 6th to 15th week of gestation.

134. The method of claim 126, wherein said trophoblast-containing cell sample is obtained from a pregnant woman at 5th to 15th week of gestation.

135. The method of claim 126, wherein said immunologically staining is effected using an antibody directed against a trophoblast specific antigen.

136. The method of claim 135, wherein said trophoblast specific antigen is selected from the group consisting of HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

137. A method of in situ chromosomal, DNA and/or RNA analysis by hybridization of a prestained specimen of cells or tissue, comprising incubating the prestained specimen of cells or tissue in a solution containing ammonium hydroxide and thereafter incubating the prestained specimen of cells or tissue with a polynucleotide probe suitable for the in situ chromosomal, DNA or RNA analysis.

138. The method of claim 137, wherein said prestained specimen is subjected to immunological staining.

139. The method of claim 138, wherein said immunological staining is effected using an antibody directly or indirectly conjugated to an enzyme.

140. The method of claim 139, wherein said enzyme is selected from the group consisting of HRP and alkaline phosphatase.

141. The method of claim 140, wherein said HRP hydrolyses a chromogenic substrate selected from the group consisting of Nova Red, diaminobenzidine (DAB), Vector(R) SG substrate, and luminol-based chemiluminescent substrate.

142. The method of claim 140, wherein said alkaline phosphatase hydrolyses a chromogenic substrate selected from the group consisting of AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate.

143. The method of claim 137, wherein said prestained specimen is subjected to activity staining.

144. The method of claim 143, wherein said activity staining is effected using a chromogenic substrate.

145. The method of claim 144, wherein said chromogenic substrate is selected from the group consisting of Nova Red, diaminobenzidine (DAB), Vector(R) SG substrate, luminol-based chemiluminescent substrate, AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate.

146. The method of claim 137, wherein said prestained specimen is subjected to cytological staining.

147. The method of claim 146, wherein said cytological staining is effected using a staining selected from the group consisting of May-Grünwald-Giemsa, Giemsa, Papanicolau, Hematoxylin, and Hematoxylin-Eosin.

148. The method of claim 137, wherein said in situ chromosomal and/or DNA analysis is effected by fluorescent in situ hybridization (FISH), primed in situ labeling (PRINS), multicolor-banding (MCB) and/or quantitative FISH (Q-FISH).

149. The method of claim 148, wherein said Q-FISH is effected using a peptide nucleic acid (PNA) oligonucleotide probe.

150. The method of claim 137, wherein said incubating the prestained specimen of cells or tissue in said solution containing ammonium hydroxide is effected for at least 2 seconds.

151. The method of claim 137, wherein said incubating the prestained specimen of cells or tissue in said solution containing ammonium hydroxide is effected for at least 1 minute.

152. The method of claim 137, wherein said incubating the prestained specimen of cells or tissue in said solution containing ammonium hydroxide is effected for a time period selected from the range of 1 second to 180 minutes.

153. The method of claim 137, wherein said incubating the prestained specimen of cells or tissue in said solution containing ammonium hydroxide is effected for a time period selected from the range of 1 minute to 60 minutes.

154. The method of claim 137, wherein said incubating the prestained specimen of cells or tissue in said solution containing ammonium hydroxide is effected for 45 minutes.

155. The method of claim 137, wherein said ammonium hydroxide is provided at a concentration selected from the range of 0.1 to 28%.

156. The method of claim 137, wherein said ammonium hydroxide is provided at a concentration selected from the range of 0.1% to 5%.

157. The method of claim 137, wherein said ammonium hydroxide is provided at a concentration of 2%.

158. A method of identifying an embryonic cell in a mixed cell population, the method comprising identifying a cell exhibiting:
(i) a nucleus/cytoplasm ratio of at least 1:1; and
(ii) at least variably condensed chromatin, thereby identifying the embryonic cell in the mixed cell population.

159. The method of claim 158, further comprising evaluating at least one morphological criterion selected from the group consisting of nuclei multiplicity, nuclei arrangement, nucleus shape, cytoplasm condensation, cytoplasm shape, and cytoplasm/nucleus orientation.

160. The method of claim 159, wherein said embryonic cell is extravillous trophoblast type I and whereas said nucleus shape is egg-shape, round or amorphy, said cell exhibits a variably condensed chromatin, said cytoplasm condensation is homogenously condensed and said cytoplasm/nucleus orientation is such that said cytoplasm encompasses 50-85% of said nucleus, thereby identifying the embryonic cells in the mixed cell population.

161. The method of claim 159, wherein said embryonic cell is extravillous trophoblast type II and whereas said nucleus shape is egg-shape, round, or amorphy, said nucleus multiplicity is two nuclei, said cell exhibits a homogenously condensed chromatin, said nucleus/cytoplasm ratio is at least 2:1, said cytoplasm condensation is homogenously condensed, and said cytoplasm/nucleus orientation is such that said cytoplasm encompasses 50-85% of said nucleus, thereby identifying the embryonic cells in the mixed cell population.

162. The method of claim 159, wherein said embryonic cell is extravillous trophoblast type III and whereas said nucleus shape is round or egg-shape, said cell exhibits a homogenously condensed chromatin, said nucleus/cytoplasm ratio is at least 2:1, respectively, said cytoplasm condensation is homogenously condensed, and said cytoplasm/nucleus orientation is such that said cytoplasm is basal to said nucleus and encompasses about 50% of said nucleus, thereby identifying the embryonic cells in the mixed cell population.

163. The method of claim 159, wherein said embryonic cell is extravillous trophoblast type IV and whereas said nucleus shape is horseshoe-shape, said cell exhibits a homogenously condensed chromatin, said cytoplasm shape is fluffy, said nucleus/cytoplasm ratio is about 1:1, said cytoplasm condensation is homogenously condensed, and said cytoplasm/nucleus orientation is such that said cytoplasm encompasses 50-80% of said nucleus, thereby identifying the embryonic cells in the mixed cell population.

164. The method of claim 159, wherein said embryonic cell is extravillous trophoblast type V and whereas said nucleus multiplicity is two nuclei, said cell exhibits a homogenously condensed chromatin, said nucleus/cytoplasm ratio is about 1:1, said cytoplasm condensation is variably condensed, and said cytoplasm/nucleus orientation is such that said cytoplasm encompasses 70-100% of said two nuclei, thereby identifying the embryonic cells in the mixed cell population.

165. The method of claim 159, wherein said embryonic cell is extravillous trophoblast clump type I and whereas said nucleus multiplicity is more than two, nucleus shape is round, egg-shape or amorphy, said cell exhibits a variably condensed chromatin, said nuclei arrangement is random, said cytoplasm shape is fluffy, said cytoplasm condensation is homogenously condensed, thereby identifying the embryonic cells in the mixed cell population.

166. The method of claim 159, wherein said embryonic cell is extravillous trophoblast clump type II and whereas said nucleus multiplicity is more than two, nucleus shape is variable, said cell exhibits a homogenously condensed chromatin, said cytoplasm shape is fluffy, said cytoplasm condensation is variable, said nuclei arrangement is in a row, thereby identifying the embryonic cells in the mixed cell population.

167. The method of claim 159, wherein said embryonic cell is syncytiotrophoblast and whereas said nucleus multiplicity is more than 10, nucleus shape is variable, said cell exhibits a homogenously condensed chromatin, said cytoplasm shape is fluffy with a well-defined cytoplasm border, said cytoplasm condensation is variable, said nuclei arrangement is random, and said cytoplasm/nucleus orientation is such that said cytoplasm is common to said nuclei, thereby identifying the embryonic cells in the mixed cell population.

168. The method of claim 159 wherein said evaluating said at least one morphological criterion is effected by staining.

169. The method of claim 168, wherein said staining is selected from the group consisting of a cytological staining, an activity staining and/or an immunological staining.

170. The method of claim 169, wherein said immunological staining is effected using an antibody directed against a trophoblast specific antigen.

171. The method of claim 170, wherein said trophoblast specific antigen is selected from the group consisting of HLA-G, PLAP, MCAM, laeverin, H315 antigen, the FT1.41.1 antigen, the NDOG-1 antigen, the NDOG-5 antigen, the BC1 antigen, the AB-154 antigen, the AB-340 antigen PAR-1, Glut-12, factor XIII, hPLH, HLA-C, JunD, Fra2, NDPK-A, Tapasin, CAR, HASH2, αHCG, IGF-II, PAI-1, p57(KIP2), PP5, PLAC1, PLAC8 and PLAC9.

172. The method of claim 169, wherein said activity staining is effected using a chromogenic substrate.

173. The method of claim 172, wherein said chromogenic substrate is selected from the group consisting of Nova Red, diaminobenzidine (DAB), Vector(R) SG substrate, luminol-based chemiluminescent substrate, AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate.

174. The method of claim 169, wherein said cytological staining is selected from the group consisting of May-Grünwald-Giemsa, Giemsa, Papanicolau, Hematoxylin, and Hematoxylin-Eosin.
